(19) United States
(12) Patent Application Publication
(10) Pub. No.: US 2011/0152166 A1
(43) Pub. Date: Jun. 23, 2011

(54) METHOD OF MODULATION OF PROTEIN PHOSPHORYLATION-DEPENDENT CONFORMATIONAL TRANSITIONS WITH LOW MOLECULAR WEIGHT COMPOUNDS

(75) Inventors: Ricardo M. Biondi, Homburg (DE); Mathias Engel, Zweibrucken (DE); Frodin Morten, Copenhagen (DK)

(73) Assignee: Ricardo M. Biondi, Homburg (DE)

(21) Appl. No.: 12/441,890

(22) PCT Filed: Aug. 20, 2007
(86) PCT No.: PCT/EP07/07424

§ 371 Date: Dec. 11, 2009

(30) Foreign Application Priority Data
Aug. 18, 2006 (EP) 06076602.9
Aug. 18, 2006 (EP) 06090142.8

(51) Int. Cl. A61K 38/02; G01N 33/573; G01N 33/574; C12N 9/12; C07C 323/56; C40B 30/02; C12Q 1/48; C12Q 1/68; A61P 35/00; A61P 3/10; A61P 3/00

(52) U.S. Cl. 514/1.1; 435/7.1; 435/194; 562/426; 506/8; 435/15; 435/6.11

(57) ABSTRACT

The present patent application discloses a method of identifying or validating a compound that modulates the phosphorylation-dependent activity of a target protein or protein complex, where the target protein or protein complex activity is regulated by phosphorylation, as well as the use of identified compounds for the production of a pharmaceutical preparation especially for the treatment of cancer, insulin resistance and diabetes.

### BACKGROUND OF THE INVENTION

Protein phosphorylation is key to the regulation of cells and organisms. Protein phosphorylation is present in bacteria (prokaryots), as well as in eukaryotic cells, it is present in unicellular organisms as well as multicellular organisms.

A protein is said to be phosphorylated when the polypeptide is post-translationally modified in a way that covalently binds a phosphate. Phosphorylation of proteins mostly is known to occur in Histidine, Aspartic acid, Serine, Threonine and Tyrosine aminoacid residues. Phosphorylation at Histidine and Aspartic acid residues can be found on proteins involved in two-component systems, frequently found in prokaryots as a signal transduction system (although also described in eukaryotic systems). Phosphorylation of Histidine residues is also found in metabolic enzymes, occasionally as a high energy intermediate of a reaction (such as in the ubiquitous enzyme nucleoside diphosphate kinase). Serine, Threonine and Tyrosine phosphorylation occurs widely in mammalian cells and is also present in prokaryots.

Protein phosphorylation is widely catalyzed by enzymes termed “protein kinases” and the protein dephosphorylation by “phosphatises”. The level of phosphorylation of a protein is therefore regulated by the activity of protein kinases and phosphatises on a specific site. In mammals, the traditional protein kinases are grouped in the “kinome” which consists of more than 500 protein kinases in the human genome and over 120 protein kinases in the yeast S. cereviceae genome. The traditional protein kinases phosphorylate Ser, Thr and Tyr residues. Protein kinases from prokaryots are less well characterized. A recent study in Basilus subtilis identified 78 phosphorylation sites: 54 on serine, 16 on threonine, and eight on tyrosine. Detected phosphoproteins were involved in a wide variety of metabolic processes but are enriched in carbohydrate metabolism. The authors reported phosphorylation sites on almost all glycolytic and tricarboxylic acid cycle enzymes, several kinases, and members of the phosphoenolpyruvate-dependent phosphotransferase system (Mol Cell Proteomics. 2007 April; 6(4):697-707). The genome of this organism appears to code for only two protein kinases with homology to the traditional type found in mammals, but non-related protein kinases have also been described. Eleven traditional protein kinases are coded by the genome of the mycobacteria which produces tuberculosis and at least one of them is required for growth. Thus, there is evidence that protein phosphorylation exists in prokaryots and that it serves for the regulation of important cellular events, such as metabolism and growth. In eukaryots a large amount of information indicates that protein phosphorylation is essential to transduce at least partly in almost all signalling events in cells. Recent studies in cells culture have concluded that a large proportion of cellular proteins are phosphorylated (Olsen et al. Cell, 2006, 127, 635-648. Thus, the authors identified 2244 HeLa cells proteins to be phosphorylated, totaling 6600 phosphorylation sites. In addition to protein kinase cascades, the targets of reversible phosphorylation included, between others, ubiquitin ligases, guanine nucleotide exchange factors and numerous different transcriptional regulators. The importance of protein phosphorylation in mammals is highlighted by the fact that alteration in the activity of protein kinases can lead to disease states such as cancer, neurological disorders or diabetes. Therefore, in order to treat human diseases, the protein kinases group has emerged as an important drug target class, comprising more than 30% of new drug targets in pharmaceutical industry (Cohen, 2002, Nat. Rev. Drug Discov. 4, 309-315).

Altogether, current data suggests that protein phosphorylation is key to many physiological events in prokaryots and eukaryots, and that pharmacological modulation of phosphorylation-dependent activities would be advantageous for the treatment of human diseases, by directly affecting the phosphorylation-dependent activities of human proteins and by directly modulating the phosphorylation-dependent activities of key proteins from infectious organisms including eukaryotic parasites, fungal organisms, bacteria or viruses.

When a protein is phosphorylated, different mechanisms may operate to transduce the phosphorylation event into a physiological response:

a—promote protein-protein interaction by means of modular phosphorylation-dependent binding domains,

b—promote conformational changes independently from the direct binding of phosphorylated residues to modular phosphorylation-dependent binding domains.

In relation to a) a number of modular domains able to specifically interact with phosphorylated sequences have been described (e.g., 14-3-3; SH2; PTB domains). Blocking the binding sites on these domains are known to block physiological interactions and are considered as possible drug targets to affect phosphorylation-dependent activities of proteins. However, most proteins being phosphorylated do not possess phosphate binding modular domains. Also, it is not expected that most phosphorylation sites would physiologically bind to modular domains. In addition, the given domains are not necessarily present in all organisms that have regulatory protein phosphorylations.

Thus, it is state of the art to affect phosphorylation-dependent events by blocking of modular phosphorylation-dependent binding domains.

The conformational changes prompted by b) relate firstly to conformational changes that promote interactions (leading to disorder-order conformational changes), and secondly to conformational changes which disrupt interactions (leading to order-disorder conformational changes/or protein-protein dissociations).

In spite of the potential to exploit phosphorylation-dependent conformational changes for drug discovery, the possibility to modulate phosphorylation-dependent conformational changes in proteins lacking modular phosphorylation-dependent binding domains remains unexplored, and has never been proven to work with small molecular weight compounds. Moreover, the molecular mechanisms which triggers conformational changes are widely unknown. In one example for which a model exists since 1989 (Barford and Johnson, (the allosteric transition of glycogen phosphorylase, Nature, 1989, 340(6235):609-16), a strategy for pharmacological modulation of the conformational change has not been suggested.

The present invention provides methodologies which allow the screening and rational development of small compounds that enable the pharmacological regulation of phosphorylation-dependent changes in proteins.

We herein provide a proof that small compounds can be “rationally” developed to regulate protein conformational changes and interactions physiologically regulated by phosphorylation. The present invention sets the basis for the development of small compounds and drugs to target phosphorylation-dependent conformational changes. This has applications in drug discovery on proteins which are phosphorylated. Most importantly, phosphorylated proteins which were never considered as drug targets could now be considered as possible drug targets by means of the methods for drug development here described.

As part of the present invention, we identified that a phosphorylation-dependent conformational change can be thought-of as a “regulated” low affinity interaction between the phosphorylated polypeptide and the target protein. Importantly, we present evidence that the phosphorylation-dependent interactions can be mimicked by small compounds and also that the small compounds can displace the in vivo interaction. Thus, an essential part of our invention is the finding that small compounds (of “drug-like” size and properties) can displace a phosphorylation-dependent interaction. Thus, we empirically found that the phosphorylation-dependent interactions are of sufficiently low affinity that are suitable for being targeted by drugs. This finding should support drug developments since, based on our invention, drug development efforts should be preferably devoted to phosphorylation-dependent interactions.

We further provide an example of the identification of a phosphorylation-dependent conformational transition, the method to identify the residues involved in a non-standard phosphate binding site and the effect of the phosphroylation on the conformational transition of the protein (AGC protein kinases other than PDK1). Furthermore, we describe that mutations at the “turn-motif” Z-phosphate binding site can both promote activation of the kinase (as found for PKB/Akt and MSK) or inhibition of the kinase (as found in other AGC kinases).

Altogether our invention provides evidence that the small compounds developed to modulate conformational changes in proteins can lead in cells to different effects as observed in vitro. Thus, compounds which prompt a particular conformation of a protein (e.g. activate an enzyme) in vitro may act in the opposite manner (e.g. as “inhibitors”) in vivo. Alternatively, compounds which prompt a particular phosphorylation-dependent conformation on a protein (e.g. considered to “inhibit” an enzyme) may act in the opposite manner in vivo. We describe that one reason for this unexpected result is that the compounds may affect the level of phosphorylation of the protein target.

The present invention allows to approach the discovery of small compounds which bind to proteins mimicking phosphorylation-dependent conformational changes that could be developed into drugs for treatment of various diseases, such as cancer, insulin resistance, diabetes, neurological disorders, stroke, depression, hypertension, metabolic syndromes, brain function, etc.

It is known that protein kinases may have multiple substrates; thus, activating or inhibiting a given protein kinase will have pleiotropic effects on a number of protein substrates. The possibility of targeting with drugs one specific phosphorylation-dependent conformational transition in a protein substrate of a protein kinase would enable the development of far more specific drugs. Since much of the future drugs will be employed in “personalized” treatments, the availability of more specific drugs will allow the more specific treatment according to the specific requirement of a patient.

The present invention also shows that the conformational transition which activates an enzyme in vitro may act as an inhibitor of the activity of the enzyme in two ways. Firstly, the compound will be an inhibitor if the drug target pocket is required for the docking of a substrate. Thus, if the substrate binding site is occupied by the compound, the substrate phosphorylation will be inhibited even if the compound mimics an activated protein conformation on the target protein. Alternatively, depending on the specific molecular mechanisms that take place in cells, compounds which activate an enzyme in vitro may displace an intramolecular polypeptide interaction from the compound binding site. In this scenario, even if the target enzyme is activated in vitro, the displacement of the intramolecular polypeptide interaction may prompt in vivo the phosphorylation or dephosphorylation of the polypeptide and transduce a physiological message different from activation. The invention also contemplates that the binding of a compound that triggers a phosphorylation-dephosphorylation conformational transition can prompt the proteolysis of the target protein in vivo, independent of the possible effect of the compound in vitro.

In a particular case, this has application is related to protein kinases which are targets for numerous disorders, including cancer, neurodegenerative disorders, diabetes, inflammation, fungal infections, parasitic infections, etc. In a more specific case, the examples refer to protein kinases from the AGC group of protein kinases. Protein kinases are enzymes that catalyze the transfer of the γ-phosphate group of ATP to serine, threonine or tyrosine residues in proteins or peptides (called substrates) in order to alter their properties. Protein phosphorylation is the most general regulatory mechanism in eukaryotic cells and regulates most fundamental as well as specialized cellular processes. Humans contain about 500 different protein kinases.

AGC kinases constitute a subfamily of protein kinases that include about 60 members. Among these, a subgroup, here referred to as the “growth factor-activated AGC kinases” is activated by insulin, growth factors, many polypeptide hormones and other extracellular stimuli. This group regulates cellular division, growth, differentiation, survival, metabolism, motility and function and it includes the kinases: protein kinase B (PKBα-γ or AKT1-3), p70 ribosomal S6 kinase (S6K1,2), p90 ribosomal S6 kinase (RSK1-4), mitogen- and stress-activated protein kinase (MSK1,2), serum- and gluticocoid-induced kinase (SGK1-3) and several members of the protein kinase C (PKC).

The regulatory PIF-pocket site the protein kinase PDK1 is the target site of phosphorylation-dependent conformational by the small compounds described in this application. It is envisaged that the compounds targeting this site on PDK1 may be employed for the treatment of cancers since they are expected to block the activation of protein kinases which are involved in cancers, such as S6K, RSK, SGK, PKCs, etc. It is expected that to achieve such results, the PIF-pocket of PDK1 may require to be blocked in a constitutive manner; for this, it is preferred that small compounds with slow off-rate are selected and developed into drugs. However, it can be envisaged that transient blockage of the pocket, may block transient activation of the substrate S6K and is expected to block a feed-back loop phosphorylation of IRS1; in such scenario, the bock of PDK1 PIF-pocket may sensitize cells for insulin signalling. Compounds acting in this way may be selected for treatment of insulin resistance or diabetes. It is further envisaged that such compounds may be of use in other circumstances where the block of transient PDK1 PIF-pocket-dependent phosphorylations may be required. It is expected that treatment for insulin resistance or diabetes may not require complete blockage of the pocket in a constitutive manner, but rather with a transient pharmacological profile that would favour the action of insulin after food intake.

Growth factor-activated AGC kinases as drug targets. The growth factor-activated AGC kinases are known or assumed to be important in a variety of important human diseases, and several of the kinases are reportedly included in drug development programs (e.g. PKB and PKC isoforms). Cancer: Most of the growth factor-activated AGC kinases are constitutively activated in cancer cells, due to hyperactivation of upstream activating pathways, and are known (PKB, S6K, PKC, RSK) or thought/hypothesized (SGK, MSK) to promote cancer cell growth, survival or metastasis. Drugs that inhibit these kinases may therefore be new anti-cancer drugs. Diabetes mellitus: The activation of PKB, a key mediator of insulin metabolic regulation, is reduced in type-II diabetes due to insulin resistance. Interference with S6K (by gene knockout) protects mice from dietary-induced diabetes. Activators of PKB or inhibitors of S6K may therefore be used as anti-type-II diabetes drugs. Hypertension: Hyperactivation of SGK is thought to promote hypertension. Compounds that inhibit SGK, may be used as anti-hypertensive drugs. Tuberous sclerosis complex syndrome (TSC): Inactivating mutations in the TSC genes results in hyperactivation of S6K, which is likely important in development of TSC. Inhibitors of S6K may therefore be used to treat TSC patients, for which currently no treatment exists. Other diseases in which AGC kinase inhibitors/activators may be used include chronic inflammation/arthritis, cardiac hypertrophy, neurodegenerative disorders and more.

Protein kinases are only a subgroup of protein targets which are regulated by phosphorylation and may be targeted using the present invention. In addition to protein kinases, other examples of proteins regulated by phosphorylation include metabolic enzymes such as pyruvate kinase (which may be a drug target for treatment of cancers) or glycogen synthase (which may be targeted for disorders in glycogen metabolism); ion channels such as the renal outer-medullary K+ channel (ROMK; Kir1.1) and the cardiac L-type Ca+ channel (the latter being a drug target for the treatment of coronary heart disease); ubiquitin ligases, including E1, E2, and E3-type enzymes (drug targets for oncology, inflammation, virology and metabolism); guanine nucleotide exchange factors (drug targets for oncology and to modulate different signalling pathways which may require modulation in inflammation, neurological disorders, diabetes, etc.) and numerous different transcriptional regulators, including transcription factors (which are specifically participating in the regulation of transcription in a large range of physiological conditions which may require modulation for treatment of cancers, diabetes, inflammation, viral infection, metabolic disorders, neurological disorders, etc.).), e.g. NFAT and NFkappaB (Rel A/p65 subunit).

### SUMMARY OF THE INVENTION

Phosphorylation/protein conformation/drug development. In a simplified-generalized model, we have identified that at least two sites are important to regulate the protein conformation by phosphorylation:

1—at least one phosphate binding site, which, in phosphorylation-dependent conformational changes provides the extra binding energy which regulates the interaction and

2—a pocket, which docks a site distinct from the phosphorylation site, and provides binding energy which may act in concert with the phosphorylation site.

The existence of 1 and 2 is the minimal requirement to allow the screening of compounds which can modulate the phosphorylation-dependent conformational change.

For example, the screenings could involve one of the following:
a first polypeptide (protein) comprising the pocket, and a phosphate binding site and a second separate polypeptide that comprises the phosphorylation site and a second site which docks to the pocket. The assay systems would aim at finding compounds which block the interaction.
it is preferred that the pocket interacts with hydrophobic aminoacids within the second polypeptide and that mutation of the said hydrophobic aminoacids block interaction with the first polypeptide.
screening systems in the presence of a first polypeptide comprising the pocket mutated at the pocket site and compounds which affect the interaction with the second polypeptide. This assay is intended to select for compounds which affect the interaction with the wild type polypeptide but not to the mutant polypeptide. (this is what happens with mutant Val127Leu in PDK1).
in silico screening of pockets, distinct from phosphate binding sites, which provide binding energy and act in concert with the phosphorylation site, where compounds predicted to bind to those sites, and affect phosphorylation-dependent changes are selected.

A central aspect of the invention is a method of identifying or validating a compound that modulates the phosphorylation-dependent activity of a target protein or protein complex, where the target protein or protein complex activity is regulated by phosphorylation, and where the target protein or protein complex contains at least two interaction sites, one phosphate binding site and a separate target site, wherein polypeptide interaction to the interaction sites are regulated by phosphorylation, and the ability of a compound to inhibit, promote or mimic the interaction to the target site is measured and a compound that inhibits, promotes or mimics the said interaction is selected, whereas when the target protein is an AGC kinase, the polypeptide interacting to the target site does not comprise the sequence Phe/Tyr-Xaa-Xaa-Phe/Tyr or comprises a mutation equivalent to Val127Leu in PDK1.

In addition, the invention provides a PDK1 protein with Val127 mutation. In the cases where the protein kinase is an AGC kinase and the screening may or may not involve a polypeptide comprising the sequence Phe/Tyr-Xaa-Xaa-Phe/Tyr, we discovered that a point mutation in the pocket termed “PIF-binding pocket” in PDK1 can serve to perform validation and screenings of compounds which interact with this site. There have been other mutations identified in the pocket to date. Nevertheless, those mutations affected the binding of hydrophobic motif polypeptides. Our unexpected finding is that the Val127Leu mutation (human PDK1 numbering) still binds hydrophobic motif polypeptides and is able to phosphorylate substrates which require docking to the pocket. Most importantly, even if it is still functional in its physiologic function, this mutant is resistant to small compounds which dock to the PIF-binding pocket. Therefore, it appears as a suitable screening tool and as a validation tool, both in vitro and in vivo (including work with knock-in animals). In addition, as the PIF-binding pocket is conserved in AGC kinases, so equivalent mutations should be protected in other AGC kinases. The mutation can be employed in constructs or mutants of PDK1 with lower than 55% identity to human PDK1 and lacking residues extensively conserved in PDK1 homologues from different organisms. The only requirement for the usage of PDK1-like polypeptides in such screenings is that the protein not mutated at the Val127 residue can change a biochemical measurable property upon binding of polypeptides and compounds and that the mutant PDK1 Val127Leu still shows such property upon binding of polypeptides but does not show such effect upon binding of small compounds.

A further aspect of the invention is the use of AGC kinases mutated at a residue equivalent to Val127 to a Leu residue or a larger residue which shows a similar effect as Val127Leu on PDK1 protein.

Another aspect of the invention is the test of compounds in a suitable organism model of disease where the experimental organism has at least one copy of the target pocket within the target polypeptide mutated as a control of the specificity of compounds. When the protein is PDK1 or an AGC kinase the experimental organism to be tested with compounds can have at least one copy of the target protein kinase gene mutated at the residue equivalent to Val127 and the effect of compounds on the organism compared with a control organism which does not have the Val127 mutated. It should be noted that PDK1 or AGC kinase mutants at Val127 to Met, Phe or Tyr may also provide an effect similar to Val127 and may be used.

Preferred organisms models of disease can be any suitable eukaryotic organism, such as the amoeba Dictiostelium discoideum, fungals such as Sacharomyces cereviceae, Candida albicans, insects such as Drosophila, worms such as C. elegans. It is also preferred that the model organism of disease is a mammal, for example a mouse. Numerous human diseases can be mimicked in mouse models. In particular, cancer models, insulin resistance, diabetic models and hypertension models are preferred. Mouse models of cancer could have PTEN mutated or may express or overexpress active protein kinases, such as PKB.

It is appreciated from our research that the identification of compounds that target one particular protein family member which is regulated by phosphorylation may lead to the identification of families of compounds which can target proteins from the same family or related families. Therefore, an aspect of the invention comprises the screening of compounds identified to affect the phosphorylation-dependent conformation on a protein family member to other related protein family members—either known to be regulated by phosphorylation or not—. It is anticipated that the in vitro or in vivo effect of those compounds could be similar or different in a related protein family members. Thus, in the example 1 we provide evidence that compounds which activate protein kinase PDK1 can be inhibitors of other AGC kinases and in example 2 we provide genetic evidence that displacement of the phosphorylated polypeptide by mutation of the “turn-motif”/Z-phosphate binding site (which could be mimicked by suitable identified compounds) can inhibit some AGC kinases, but also activate PKB/Akt (this is also the case for MSK1, not shown).

In addition, another aspect of the invention are small compounds which can bind to the PIF-pocket of PDK1 and other AGC kinases and prompt conformational changes on the proteins as will be shown below.

A further aspect of the invention is the use of the small compounds for drug discovery, as lead compounds, to evaluate effects in cells and validate drug targets, to crystallize with AGC kinases or model onto three dimensional models of AGC protein kinases and perform structure based drug design.

A yet further aspect of the invention is the use of the small compounds as part of medicaments for treatment of human beings.

In order to employ the method of the invention, it is a requirement that the target protein is regulated either intra-molecularly or inter-molecularly by phosphorylation-dependent interactions. In example 1, the PDK1 phosphorylation-dependent conformational transition is given in-trans with polypeptides derived from substrates of PDK1; alternatively, in example 2, the phosphorylation-dependent conformational change is prompted physiologically in-cis by a phosphorylation within the AGC kinase polypeptide. The interaction between the target polypeptide and the second polypeptide can be measured directly, for example in a pull-down experiment, using surface-plasmon resonance, a fluorescence technology based on the binding and displacement of a known labelled molecule, by following intrinsic fluorescence which is sensible to binding or conformational change; alternatively, the interaction can be measured by any indirect method such as enzyme activity measurement, if the phosphorylation-dependent conformational change prompted changes in a measurable property of the activity of the target protein.

The invention contemplates that the protein target is tested for the effect on the conformation of the target protein using phosphorylated polypeptides derived from the phosphorylation-dependent interacting partner (in-trans), or derived from polypeptides derived from phosphorylation sites within the target polypeptide. It is further preferred that the polypeptides include one or more hydrophobic aminoacids. It is further preferred that the polypeptides are not derived from sequences of aminoacids predicted to form part of protein domains.

Polypeptides can be synthetic synthesized or produced as recombinant fusion proteins, for example as a fusion to GST which allow binding to glutathione resins or SNAP-tag (Covalys), which can be covalently labelled with different groups, including fluorescent groups, biotin, etc. These and other tags could facilitate measurement of binding to the target protein.

The invention further contemplates that the derived polypeptides may not be from the actual physiologically interacting partner but from the equivalent region of a protein from the same family or chimeras.

Furthermore, the invention further contemplates that the phosphorylated polypeptide may be replaced by a polypeptide which contains a Glutamic acid or an Aspartic acid instead of the phosphorylated residue to mimic the phosphorylated polypeptide or a different, non-acidic amino acid, such as Alanine or Valine, to mimic a non-phosphorylated polypeptide.

Another aspect of the invention is the use of mutants of the target polypeptide, the mutation affecting the target pocket.

Another aspect of the invention is the use of mutants of the target polypeptide at the phosphate binding site within the target polypeptide. The invention contemplates that the phosphate binding site can be probed by modelling the target protein and selecting for areas within the surface of the protein where one or more positively charged residues or Glutamine, Asparagine or Histidine residues are located within a restricted space suitable for phosphate binding. Alternatively, in the absence of structural information, positively charged aminoacids can be mutated and the phosphor-peptide effects compared between the non-mutated and the Arg/Lys mutated forms of the target protein.

The invention further identified that, mutation of phosphate-binding site residues can lead to inhibition of protein activity or uncontrolled activation of protein activity. Since in disease related proteins such mutations can lead to disease states, the invention further contemplates the use of this information for genetic screenings for mutatins of AGC kinases turn-motif/Z-phosphate binding site residues. For example, the invention contemplates the screening of mutations at the PKB turn-motif/Z-phosphate binding in samples from patients. Mutations in these residues may correlate with increased PKB activity and cell survival, which could prompt cell survival and favour cancer development in cancer tissues. Based on the results of the screening a patient may be treated with PKB inhibitors and not with upstream inhibitors such as EGFR or PDK1 inhibitors. Similarly, mutations in MSK prompted increased kinase activity. Thus, screenings in mutations in MSK could help to determine the source of a disease and plan the appropriate treatment of a patient.

A major problem in generating screening systems for phosphorylation-dependent regulatory sites is that the affinity of interacting polypeptides may be usually low affinity. Therefore screenings and interactions assays suitable for low affinity interactions are preferred. The assay system should be defined based on the affinity of the interaction and characteristics of the targe protein. For example, it may be often preferred that in the case when the target protein is an enzyme, an activity assay is employed.

Aspects of the invention are further illustrated in the Example 1 and 2 below.

### Example 1

### Method of Modulation of Protein Phosphorylation-Dependent Conformational Transitions with Low Molecular Weight Compounds (Below)

### Example 2

### A Method of Activation of AGC Kinases by Linker and Hydrophobic Motif Phosphorylation Sites (Below)

### Introduction to Example 1

50 years after its discovery, protein phosphorylation is the most widely studied intracellular regulatory mechanism (Pawson and Scott, 2005). Phosphorylation of proteins often induces conformational changes with physiological outcomes, such as increased or decreased activity of enzymes. Phosphorylation-mediated conformational transitions are likely to be of general relevance, since it is estimated that up to one third of cellular proteins are phosphorylated. Protein phosphorylation is catalysed by protein kinases, which transfer the terminal phosphate from an NTP (generally ATP) to substrate proteins. In fact, protein kinases are often regulated by phosphorylation, which triggers conformational changes in their catalytic domains (Huse and Kuriyan, 2002). As deregulation of protein kinases can lead to disorders such as cancer (Blume-Jensen and Hunter, 2001), they have emerged as one of the major groups of drug targets in the pharmaceutical industry (Cohen, 2002).

We and others have previously gained insight into the biochemical, molecular and structural aspects of the mechanism by which a family of protein kinases termed AGC kinases are regulated via phosphorylation within a hydrophobic motif (HM, Phe-Xaa-Xaa-Phe-Ser/Thr(P)-Tyr), which is usually located 45-60 residues C-terminal to the protein kinase catalytic core (Biondi and Nebreda, 2003; Etchebehere et al., 1997; Newton, 2003a; Parker and Parkinson, 2001; Pearl and Barford, 2002). In AGC kinases, the HM phosphorylation site acts in concert with the “activation loop” phosphorylation site to stabilize the active conformation. The mechanism by which HM phosphorylation triggers activation relies on the docking of the phosphorylated HM to a particular HM binding pocket in the protein kinase catalytic domain. The HM binding pocket was first defined in the cAMP dependent protein kinase (PKA) structure (Knighton et al., 1991) (FIG. 1A). In the phosphoinositide-dependent protein kinase 1 (PDK1) the pocket was characterized as a regulatory site and was termed the “PIF-pocket” (Biondi et al., 2000). In PDK1, the HM/PIF-pocket docks the HM of substrate protein kinases, e.g. RSK, S6K, SGK, only when they are phosphorylated. This interaction not only provides docking for the substrates, but it also activates PDK1 to enable phosphorylation, and hence activation of RSK, S6K and SGK (Biondi et al., 2001; Collins et al., 2003; Frodin et al., 2000). The equivalent HM/PIF-pocket was subsequently found to be a regulatory site in many AGC kinases (Frodin et al., 2002; Yang et al., 2002). Significantly, inactive structures of the AGC kinases PKB and MSK show that the HM-pocket is disturbed; two of its lining walls, the conserved α-C helix and the α-B helix, are either disordered or replaced by an unusual β-sheet (Huang et al., 2003; Smith et al., 2004; Yang et al., 2002). Concomitant to this change in the HM/PIF-pocket, the structure of the ATP binding site in the inactive catalytic domains of AGC kinases is significantly different from that observed in the active kinase. In order to activate the kinases, the binding of the phosphorylated HM sequence (P-HM) to the HM/PIF-pocket regulatory site must bring about a conformational change, which directly involves the HM/PIF-pocket and allosterically affects the ATP binding site. Thus, the inactive-active transition involves a phosphorylation-dependent conformational change (FIG. 1E). The model of allostery between the active site and the regulatory site suggests that the interaction of a phosphorylated HM to the HM/PIF-pocket activates PDK1 by stabilizing the □-C helix in the active form. In this process, a conserved Glu residue (Glu130 in PDK1) correctly positions a key active site Lys residue (Lys111 in PDK1) which directly interacts with the phosphate from ATP (Biondi, 2004; Biondi et al., 2002; Pearl and Barford, 2002). Biochemical and structural studies on PDK1 and AGC kinases have defined a phosphate binding site next to the HM/PIF-pocket (Biondi et al., 2002; Frodin et al., 2002). This phosphate-binding site is responsible for triggering the binding of the HM to the HM/PIF-pocket only when it is phosphorylated. However, overexpression of PDK1 can trigger the phosphorylation of substrates which lack the HM sequences (Frodin et al., 2002), indicating a subtle regulation of the interaction. For this reason, the requirement of the HM/PIF-pocket in PDK1 for inter-molecular interactions with substrates in vivo has been studied using knock-in cell lines where the levels of PDK1 are kept at physiological levels (Collins et al., 2003; Collins et al., 2005). On the other hand, the regulation by HM phosphorylation in AGC kinases can be viewed as a particular example of an induced intra-molecular interaction regulated by the presence of the phosphate.

Other well known molecular mechanisms of regulation by phosphorylation employ a similar intra-molecular phosphorylation-dependent interaction strategy. Such is the case of tyrosine kinases (Sicheri et al., 1997; Xu et al., 1997) and the glycogen synthase protein kinase 3 (GSK3) (Dajani et al., 2001; Frame et al., 2001), where phosphorylation at a site outside the catalytic domain triggers the intra-molecular binding of the phosphorylated sequence to a phosphate binding site in the catalytic domain and the regulation of GSK3 activity. Although the molecular events that trigger the conformational changes by phosphorylation in most proteins are not known (Johnson and Lewis, 2001), it is expected that an analogous mechanism involving intra- or inter-molecular phosphorylation-dependent docking may promote conformational changes in a larger range of proteins.

In this study, we present the rational design and characterization of small molecules that, binding to the HM/PIF-pocket regulatory site in PDK1, allosterically activate the kinase by mimicking the conformational transitions physiologically triggered by phospho-peptide docking. These findings open a novel field in the development of small modulators of protein kinase activity. Most importantly, our work has implications for modulating conformational transitions in other proteins.

One of the objects of the invention is therefore a method to activate kinases by mimicking the conformational transitions physiologically triggered by phospho-peptide docking

### Results

Discovery of Small Molecules which Increase PDK1 Activity

Phosphorylation can sometimes be mimicked by replacement of the phosphorylatable residue with an acidic residue. This occurs physiologically in the HM of the protein kinase C related protein kinase 2 (PRK2), from where the 24 amino acid polypeptide PIFtide is derived. PIFtide can activate both PDK1 and PKB with high potency (Biondi et al., 2000; Yang et al., 2002). We found that—surprisingly—other chemical groups, distinct from phosphate, may mimic the required interactions and trigger the allosteric conformational changes. In addition, PIFtide has considerably higher affinity for PDK1 than any other HM and P-HM tested (Biondi et al., 2001). However, a relatively large polypeptide comprising the complete hydrophobic motif present in PIFtide (GFRDFDY) did not activate PDK1 at concentrations up to 500 □M (data not shown). Therefore, in order to test if small compounds can trigger the phosphorylation-dependent conformational changes in AGC kinases, we next evaluated whether non-peptide small molecules could be suitable as allosteric modulators of PDK1.

As a first step in the rational design of small compounds to mimic the phosphorylation-dependent transition, we compared the structure of the PDK1 HM/PIF-pocket with that of the closed, active conformation of PKA. The HM/PIF-pocket contains two sub-pockets, where the two Phe residues from the HM dock (FIG. 1A,B). In the PDK1 structure, one of these sub-pockets appeared significantly diminished in depth due to the positioning of Phe157 (Biondi et al., 2002). In addition, even in the presence of ATP, PDK1 crystallized in an “intermediate” form, with active site residues not positioned correctly for catalysis. Therefore, to enable the development of compounds to mimic the active form of AGC kinases, we decided to use the closed, active structure of PKA as a model. We performed in silico screening of a chemical library consisting of 60,000 small molecular weight compounds and selected compounds predicted to bind to the PIF-pocket site on the active PKA structure. In particular, we focussed on the positions of the aromatic rings from Phe347 and Phe350 in PKA (FIG. 1B).

Depending on the parameters imposed, between 250 and 2500 different compounds were identified in the in silico screenings. The selected compounds were visually evaluated and 220 compounds were further tested in vitro. The results revealed that two compounds significantly increased the intrinsic activity of PDK1 towards a polypeptide substrate that comprises the activation loop residues of PKB, known as T308tide (Biondi et al., 2000). As a control, these two compounds did not modify the activity of PDK1 in the presence of an excess of the HM polypeptide, PIFtide. Based on the structure and the common characteristics of the hits, we further evaluated related small molecular weight compounds with different scaffolds and selected compound 1 (3-(p-Chlorophenyl)-3-oxo-1-phenyl)-propyl sulfanyl acetic acid) for further characterization (FIG. 1C).

Another object of the invention is therefore a screening method which identifies compounds that activates kinases. Further object of the invention is a method for rational drug design and evaluation. Also object of the invention is 3-(p-Chlorophenyl)-3-oxo-1-phenyl)-propyl sulfanyl acetic acid and its use for the manufacture of a medicament for the treatment of cancer.

### Activation of PDK1 by P-HM Polypeptides and Compound 1 Requires the Presence of a Negative Charge

The initial compounds that activated PDK1 possessed a carboxylate group. Therefore, we first characterized the requirement of the carboxylate on compound 1 and compared the results to the requirement of the phosphate on P-HM polypeptides. PDK1 was activated by polypeptides P-HM-PKB and P-HM-RSK, derived from the phosphorylated HMs of AGC kinases PKB and p90 ribosomal S6 kinase (RSK), respectively (FIG. 2A). The corresponding non-phosphorylated polypeptides did not activate PDK1, confirming that the activation of PDK1 by the HM of substrates was dependent on the phosphorylation of the HM. Similarly to P-HM polypeptides, compound 1 activated PDK1 with an AC50 of 25 μM. In contrast, a compound bearing a methyl ester instead of a free carboxylate group (compound 2) was inactive across a wide range of concentrations (FIG. 2B).

### Activation of PDK1 by Compounds is Abolished by Mutations in the HM/PIF-Pocket

We then evaluated the mode of action of compound 1 by analysing how its activating properties were affected by a series of mutations in the HM/PIF-pocket and surrounding residues. Table I summarizes the results obtained with PDK1 mutants. As previously described (Biondi et al., 2000), most PDK1 proteins mutated along the HM/PIF-pocket showed only partially reduced binding to PIFtide and could still be activated by PIFtide, although they required higher concentrations for maximal activation. We also tested the effect of P-HM-peptides and small compounds on PDK1 proteins mutated at different positions within the HM/PIF-pocket (FIG. 2C). Mutation of Gln150, Thr148 or Ile119 completely abolished the activation of PDK1 by the phosphorylated polypeptides P-HM-RSK (20 μM) and P-HM-PKB (FIG. 2C and not shown). On the other hand, as expected from the interaction with a much smaller compound, most PIF-pocket mutants were still activated by compound 1 (e.g. mutants PDK1[Gln150Ala] and PDK1[Thr148Val]). Constitutively active PDK1 mutants, in which the hydrophobic residue Leu155 is replaced with Glu or Ser (Biondi et al., 2000), could not be further activated by P-HM peptides nor by compound 1 (FIG. 2C and Table I).

Mutation of Val127 (at the base of the HM/PIF binding pocket) to Leu, abolished activation by small compounds (FIG. 2D). On the other hand, PDK1[Val127Leu] was fully activated by PIFtide and P-HM-RSK (FIG. 2E). Val127 is a non-conserved residue forming part of the base of the HM/PIF-pocket in PDK1. In PKA, its equivalent is Thr88, which is located at the base of the Phe347 subpocket (FIG. 1B). Replacement of Val127 by Thr, generated a PDK1 HM/PIF-pocket mutant that had increased specific activity (200%) and could be further activated by PIFtide (260%) and compound 1 (240%), suggesting that a Thr at this position did not abolish the ability of compounds to bind and activate the kinase. Altogether the results suggested that compound 1 targeted the intended site, the HM/PIF-pocket in PDK1 and that the identity of residues forming the hydrophobic pocket, could determine the ability of compounds to activate PDK1. Thus, our results provide evidence that the residues forming part of the HM/PIF-pocket could provide specificity to the compounds.

### PDK1 Arg131 is Required for Activation by Compound 1

Structural and biochemical studies have previously defined the residues which form the phosphate binding site responsible for docking the P-HM onto PDK1 (Biondi et al., 2002; Frodin et al., 2002). Thus, the crystal structure of PDK1 defined a sulfate binding site next to the HM/PIF-pocket, comprising Arg131, Thr148, Lys76 and Gln150. Biochemical analysis of PDK1 mutants identified Gln150 and Arg131 as the most important residues that allowed binding and promoted activity of PDK1 by the phosphorylated HM polypeptides. As described above (FIG. 2C), PDK1[Gln150Ala] and PDK1[Thr148Val] were still activated by compound 1 suggesting that the carboxylate group from compound 1 did not make any specific interactions with these residues. We next examined if the positively charged Arg131 was required for the activation by compound 1. Indeed, when Arg131 was mutated to Met or Ala, the resulting PDK1 mutant was no longer activated by compound 1 (FIG. 2E). This result indicated that the activation of the enzyme by compound 1 required the positive charge from Arg131.

### The Activity of a PDK1 50-360[CT-PIF] Chimera is not Affected by Compound 1.

We generated a PDK1 chimera consisting of the catalytic domain of PDK1 comprising residues 50-360 joined to the last 48 aminoacids from PRK2, which include the sequence of PIFtide (PDK1 50-360[CT-PIF]). PIFtide binds with high affinity to PDK1. Therefore, we expected that the HM/PIF-pocket in this protein would be strongly bound to the C-terminus of PRK2. In this scenario, the specific activity of the PDK1 50-360[CT-PIF] chimera would not be affected by compounds which otherwise would bind to the PDK1 50-360 HM/PIF-pocket. The PDK1 50-360[CT-PIF] chimera had 1.5-fold higher specific activity than the PDK1 catalytic domain alone. Furthermore, the activity of PDK1 50-360[CT-PIF] chimera was not affected by PIFtide at concentrations that activated the PDK1 50-360 protein (FIG. 2G). Finally, PDK1 50-360[CT-PIF] specific activity was not affected by compound 1, further supporting the notion that PIFtide and compound 1 targeted an overlapping site in the catalytic domain of PDK1.

Compound 1 Blocks the Interaction of PDK1 with the HM-Polypeptide PIFtide

We next used surface plasmon resonance to test the ability of compound 1 to compete with the interaction between GST-PDK1 1-556 to PIFtide. We first probed the binding of PDK1 to the biotin-PIFtide coated chip at different PDK1 concentrations (FIG. 3A). The interaction of PDK1 with biotin-PIFtide indicated a K_{D} of approximately 65 nM. Pre-incubation of PDK1 with unlabelled PIFtide abolished this interaction (FIG. 3B), verifying that the PDK1-biotin-PIFtide interaction was specific. Therefore, in order to allow a sensitive measurement of any displacement of binding by small compounds, the competition experiments were performed at a PDK1 concentration of 45 nM. Under the established conditions, compound 1 was able to disrupt the binding of PDK1 to PIFtide in a concentration dependent manner (FIG. 3C). By contrast, the ester form (compound 2) did not displace the binding significantly, suggesting that it did not interact with PDK1 at the HM/PIF-pocket site with comparable affinities. These experiments further confirmed that compound 1 interacted with the PIF-binding pocket in PDK1.

### The Identity of R1, R2 and R3 Substituents in Compound 1 Confer Specificity Towards PDK1

In order to confirm that the compound action on PDK1 was specific, we then synthesized a series of related compounds containing chlorine substituents at different positions (FIG. 1D). Compounds with chlorine at R1, or in both R1 and R2 positions were capable to activate PDK1, whereas compounds with chlorine only at R2 or in the three positions R1, R2 and R3 were less active towards PDK1, or even acted as inhibitors at higher concentrations (Table II, first column). This indicated that substitutions in R1, R2 and R3 conferred specificity for PDK1. Substitutions in R1 and R2 were permissive and the resulting compounds were active towards PDK1. Thus, the specific location of the chlorine substitutions in analogue compounds had markedly different effects on PDK1 and provided evidence of a specific interaction.

The expert skilled in the art will recognize, that a number of compounds with similar substitution patterns will have similar properties towards PDK1.

Another object of the invention is therefore a compound according to general formula I

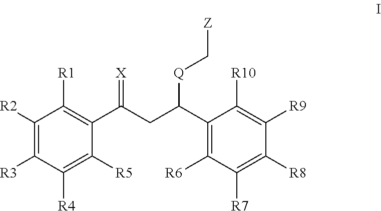

in which X is selected from O, N—R, or NO—R, R is H, C1-C4-alkyl, or —(CH₂)₁₋₄—Y, wherein Y is a functional group Q is selected from S or CH2, Z is selected from COOH, tetrazolyl, nitril, phosphonic acid, phosphate, or COOE, in which E is C1-C5-alkanoyloxy-C1-C3-alkyl or C1-C-alkoxycarbonyloxy-C1-C3-alkyl and R1, R4-R10 is selected from H, halogen, C1-C4-alkyl, C2-C4-alkenyl, or trifluoromethyl, and R2, R3 are either member of benzoanneleted cyclopentane, cyclohexane or benzene or are independently selected from H, halogen, C1-C4-alkyl, C2-C4-alkenyl, or trifluoromethyl.

A further aspect of the invention are compounds of formula I in which R1, R4-R7 and R10 is selected from H or F, and R2, R3, R8 and R9 are selected from H, halogen, C1-C4-alkyl, C2-C4-alkenyl, or trifluoromethyl, and at least one of R2, R3, R8 or R9 is not H.

A further aspect of the invention are compounds of formula I in which X is selected from O or NOH, and Z is selected from COOH or COOE in which E is C1-C5-alkanoyloxy-C1-C3-alkyl or C1-C-alkoxycarbonyloxy-C1-C3-alkyl.

A still further aspect of the invention are compounds of formula I in which R1, R4-R7, R9 and R10 is H.

A still further aspect of the invention are compounds of formula I in which X is O.

A still further aspect of the invention are compounds of formula I in which E is selected from acetoxymethyl, propionyloxymethyl, isopropionyloxymethyl, N-butyryloxymethyl, isobutyryloxymethyl, 2,2-dimethylpropionyloxymethyl, isovaleryloxymethyl, 1-acetoxy-1-ethyl, 1-acetoxy-1-propyl, 2,2-dimethylpropionyloxy-1-ethyl, 1-methoxycarbonyloxy-1-ethyl, 1-ethoxycarbonyloxy-1-ethyl, 1-isopropoxycarbonyloxyethyl or methoxycarbonyloxymethyl.

An expert skilled in the art will further recognise, that in the derivatives based on formula I where X is N—R or NO—R, R is an appropriate moiety for further derivatisation, e.g. by parallel synthesis approaches. It is evident for experts in the field that R can include more functions than an alkyl chain. Thus, R can consist of a linear spacer group such as methyl, ethyl or ethylene glycol, followed by any functional group including, but not limited to, alcohol, ester, amide, carboxyl, amine, aromatics, aromatic and aliphatic heterocycles.

Another aspect of the invention are compounds of formula II

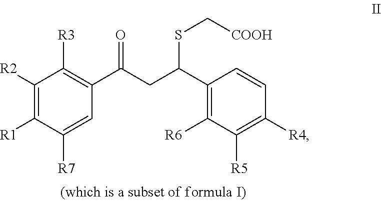

in which R1-R7 have the meanings indicated in the following table:

| Compound | |
|---|---|
| No. | |
| II.1 | R1 = Cl, R2-R7 = H |
| II.2 | R1 = Br, R2-R7 = H |
| II.3 | R1 = I, R2-R7 = H |
| II.4 | R1 = CF₃, R2-R7 = H |
| II.5 | R1 = CH₃, R2-R7 = H |
| II.6 | R1 = ethyl, R2-R7 = H |
| II.7 | R1 = propyl, R2-R7 = H |
| II.8 | R1 = isopropyl, R2- |
| | R7 = H |
| II.9 | R1 = Cl, R2 = Cl, R3- |
| | R7 = H |
| II.10 | R1 = H, R2 = Cl, R3- |
| | R7 = H |
| II.11 | R1 = Cl, R2 = Cl, |
| | R4 = Cl, |
| | R3 = R5 = R6 = R7 = H |
| II.12 | R1 = CF₃, R4 = Cl, |
| | R2 = R3 = R5 = R6 = R7 = |
| | H |
| II.13 | R1 = Cl, R4 = Cl, |
| | R2 = R3 = R5 = R6 = R7 = |
| | H |
| II.14 | R1 = Br, R4 = Cl, |
| | R2 = R3 = R5 = R6 = R7 = |
| | H |
| II.15 | R1 = I, R4 = Cl, |
| | R2 = R3 = R5 = R6 = R7 = |
| | H |
| II.16 | R1 = Cl, R4 = F, |
| | R2 = R3 = R5 = R6 = R7 = |
| | H |
| II.17 | R1 = Cl, R3 = Cl, R2 = H, R4- |
| | R7 = H |
| II.18 | R1 = H, R2 = H, R3 = Cl, R7 = Cl, |
| | R4-R6 = H |
| II.19 | R1 = Cl, R5 = Cl, R3-R4 = H, |
| | R6 = R7 = H |
| II.20 | R1 = Br, R5 = Cl, R3-R4 = H, |
| | R6 = R7 = H |
| II.21 | R1 = I, R5 = Cl, R3-R4 = H, |
| | R6 = R7 = H |
| II.22 | R1 = Cl, R2-R5 = H, R6 = F, |
| | R7 = H |
| II.23 | R1 = Br, R2-R5 = H, R6 = F, |
| | R7 = H |
| II.24 | R1 = I, R2-R5 = H, R6 = F, |
| | R7 = H |
| II.25 | R1 = Cl, R2-R5 = H, R6 = Cl, |
| | R7 = H |
| II.26 | R1 = Br, R2-R5 = H, R6 = Cl, |
| | R7 = H |
| II.27 | R1 = I, R2-R5 = H, R6 = Cl, |
| | R7 = H |
| II.28 | R1 = CF₃, R5 = Cl, R3-R4 = H, |
| | R6 = R7 = H |
| II.29 | 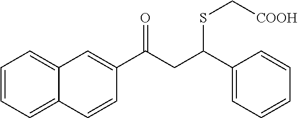 |
| II.30 | 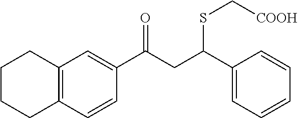 |
| II.31 | 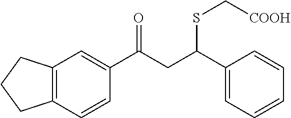 |

Another object of the invention is therefore a compound according to general formula III

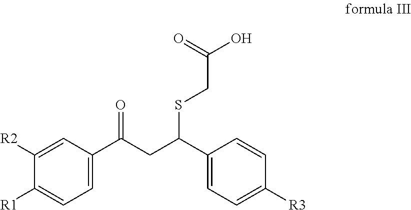

in which R1, R2 and R3 independently of each other have the meaning of a hydrogen, fluorine, chlorine, bromine, iodine atom, a nitro group, or a C1-C4 alkyl group which may be saturated or unsaturated which may be straight or branched and which may be partially or completely fluorinated.

Preferred embodiments of formula III are compounds in which at least one of R1, R2 or R3 are a chlorine atom, a methylgroup or a trifluormethylgroup.

More preferred embodiments of formula III are compounds in which R1 is a chlorine atom, a methylgroup or a trifluormethylgroup.

A still further aspect of the invention is the use of any of the compound of formulae I-III for co-crystallization with a protein.

A still further aspect of the invention is the use of any of the compound of formulae I-III for target validation studies.

A still further aspect of the invention is the use of any of the compound of formulae I-III for as a lead compound for drug development, including virtual docking to target proteins.

A still further aspect of the invention is the use of any of the compound of formulae I-III for the production of a pharmaceutical preparation.

A still further aspect of the invention is the use of compounds according to general formula I-III for the production of a pharmaceutical preparation for the treatment of cancer.

A still further aspect of the invention is the use of compounds according to general formula I-III for the production of a pharmaceutical preparation for the treatment of insulin resistance and diabetes.

Compound 1 does not Affect the Intrinsic Activity of Related AGC Kinases

Since the initial compounds were selected in silico based on properties of the HM-pocket that are common between different members of the AGC kinase family, we evaluated the protein kinase specificity of compound 1 by testing its effect on other AGC kinases (Table II A). At a concentration of 20 μM, compound 1 activated PDK1, but had no significant effect on the AGC kinases PKBa/AKT1, SGK1, PRK2, PKCζ, S6K or PKA. At higher concentrations, compound 1 did not increase the activity of any other AGC kinase tested. On the other hand, its methyl-ester form (compound 2), without the negative charge, did not affect significantly the activity of any protein kinase tested. Interestingly, a similar compound with Cl at position R2 (compound 4) or R1 and R2 (compound 3) retained the ability to activate PDK1, but also significantly inhibited other protein kinases in the panel, such as SGK and PKCζ. On the other hand, compound 5, with Cl at R1, R2 and R3 did not affect the activity of any protein kinase in the panel (Table II B). We conclude that the compounds developed to mimic the conformational transition in AGC kinases can be produced with considerable selectivity towards one AGC kinase family member.

Compound 1 Blocks the Phosphorylation and Activation of its Substrates S6K and SGK which Require the HM/PIF-Pocket for Docking

SGK and S6K are substrates of PDK1 that require the docking of their phosphorylated HM to the PDK1 HM/PIF-pocket to trigger their phosphorylation and activation by PDK1 (Biondi et al., 2001; Collins et al., 2003). Thus, based on those studies, it was expected that compounds that specifically target the HM/PIF-pocket in PDK1 would inhibit S6K and SGK phosphorylation and activation by PDK1. Therefore, we tested the effect of compound 1 on the phosphorylation and activation of S6K and SGK constructs containing a negatively charged residue in place of the HM-phosphorylation site (Thr412Glu and Ser422Asp, respectively). These kinases are activated by PDK1 phosphorylation. Compound 1 decreased the PDK1-catalyzed phosphorylation of S6K1 (FIG. 4A), in agreement with the hypothesis that compound 1 bound to the HM/PIF-pocket in PDK1. In addition, compound 1 inhibited SGK1 activation by PDK1. In contrast, SGK activation by PDK1[Val127Leu] was not inhibited by compound 1 (FIG. 4B), suggesting that the inhibitory effect was not due to an interaction between compound 1 and SGK, but rather due to the binding of compound 1 to the hydrophobic HM/PIF-pocket of PDK1.

We also attempted to verify if compound 1 could have a similar effect on S6K phosphorylation in a cell assay approach. To this end, we transfected HEK 293 cells with plasmids encoding for GST-S6K-T2[Thr412Glu] and GST-S6K-T2[Thr412Ala] and purified the fusion proteins using a glutathione-sepharose resin. In agreement with the in vitro data, treatment of cells with 200 μM of compound 1 caused a decrease of S6K1-T2[Thr412Glu] activity, which correlated with a decrease in the phosphorylation at the activation loop (FIG. 4C). We also tested the effect of compound 1 on S6K1-T2[Thr412Ala], an inactive mutant which also interacts with PDK1, albeit with lower affinity (Biondi et al., 2001). Again, we observed that the incubation of the cells with compound 1 significantly decreased the activation loop phosphorylation (FIG. 4C). A similar decrease in activity of GST-S6K-T2[Thr412Glu] was observed when the kinase assay was performed using anti-GST immunoprecipitates (FIG. 4D). The results suggested that the PDK1 HM/PIF-binding pocket is available for interaction with a small molecular weight compound within the environment of the cell. Similar results are achieved with other compounds according to general formula 1.

### Probing the Conformational Change Induced by HM-Polypeptides and Compound 1 by Use of the Fluorescent ATP Analogue TNP-ATP

Since the ATP binding site appears structurally affected in the crystals formed by inactive conformations of PKB and MSK (Huang et al., 2003; Smith et al., 2004; Yang et al., 2002), the model suggests that activation of the kinase by HM phosphorylation in those kinases must prompt conformational changes in the ATP binding site. We have already indirectly measured a conformational change in PDK1 by measuring the allosteric activation of PDK1 by compound 1. In addition, we also examined if the conformational change obtained by phosphopeptides was similar to that obtained with small compounds probing the ATP binding site conformation in solution using a fluorescent ATP analogue, trinitrophenyl-ATP (TNP-ATP). In the absence of PDK1, TNP-ATP produced approximately 1.5 arbitrary units (a.u.) of fluorescence, and the inclusion of PDK1 increased its fluorescence intensity to approximately 3.3 a.u. without change in the emission spectra. Inclusion of excess ATP (1 mM) diminished the effect, indicating that ATP competed with TNP-ATP for binding to PDK1. As an additional control, the fluorescence emission scans were performed in the absence of PDK1; PIFtide, P-HM-polypeptides and the compounds did not modify the TNP-ATP fluorescence in the absence of PDK1 (not shown). We then tested the effect of HM-polypeptides and compound 1 on the fluorescence intensity of TNP-ATP/PDK1. The addition of P-HM-polypeptides and PIFtide (FIG. 5A and not shown) decreased the intensity of TNP-ATP emitted fluorescence. Similarly, small compounds produced a concentration-dependent decrease in TNP-ATP fluorescence in the presence of PDK1 (FIG. 5B), while they had no effect on TNP-ATP fluorescence in the absence of the kinase. Thus, as measured by the fluorescence emission of TNP-ATP, HM-polypeptides and compound 1 produced similar effects on the ATP binding site of PDK1. The same applies to the other compounds of general formula I.

Mutation at Thr226 in PDK1 Uncouples the Ligand Binding from Activation

Previous work has identified some requirements for PDK1 activation by HM polypeptides. In order to shed light on the molecular requirements for the allosteric transition, we generated PDK1 proteins comprising the catalytic domain (CD) (50-360) mutated in a Phe or Thr residue located within or just next to the DFG motif. In the first mutant, the Phe residue from the DFG motif was mutated to Trp (PDK1[Phe224Trp]). In the second mutant, the Thr residue within the sequence DFGT was mutated to Trp (PDK1 [Thr226Trp]). Activity tests indicated that both PDK1 [Phe224Trp] and PDK1 [Thr226Trp] mutants had 3 fold reduced basal specific activity as compared to the wild type protein (data not shown), indicating that they were overall well folded. Further biochemical characterization revealed that the wild type protein and PDK1 [Phe224Trp] were activated by PIFtide and compound 1. In sharp contrast, PDK1 [Thr226Trp] was not activated by PIFtide or by compound 1 (FIG. 6A). Surprisingly, surface plasmon resonance experiments described below indicated that the three proteins bound to biotin-PIFtide and biotin-P-HM-S6K to similar levels. Thus, the wild type protein produced approximately 600 response units (RU) when all PIFtide binding sites on the chip were occupied (FIG. 6B), while the concentration of protein required for half maximal binding was 0.5 μM. We were not able to accurately measure the binding of PDK1 [Phe224Trp] and PDK1 [Thr226Trp] at high concentrations because they produced non-specific binding to the chip. Nevertheless, as the maximal RU is dependent on the size of the protein, the mutants should have identical maximal binding as the wild type protein. Using the same chip, we could establish that mutant proteins required only 2-4 fold higher concentration of protein to reach a binding level of approximately 250 RUs, indicating only a moderate decrease in affinity for biotin-PIFtide (FIG. 6 C-E). To further verify that the binding of PDK1 [Phe224Trp] and PDK1 [Thr226Trp] P-HM-S6K was indeed specific and with a similar affinity to the wild type protein, we pre-incubated the PDK1 proteins with distinct concentrations of PIFtide prior to injection. The results show that the measured interaction was specific to the HM/PIF-pocket and that the affinities of the two mutants for HM-polypeptides were both 3-4 fold lower than the wild type counterpart (FIG. 6C-E). Taken together with the activity data, we conclude that PDK1 [Thr226Trp] was able to bind PIFtide but was not activated by this interaction. The above results suggested that mutation of Thr226 in PDK1 does not abolish activity but abolishes the activation by PIFtide, uncoupling the binding of PIFtide from the activation mechanism.

### BRIEF DESCRIPTION OF THE DRAWINGS

FIG. 1—C-terminal hydrophobic motif and the PIF-pocket of PKA. Structure of compounds mimicking the phosphorylated hydrophobic motif. (A) The PKA catalytic domain surface structure is shown with underlying ribbon representation. The C-terminal Phe-X-X-Phe-COOH residues and the ATP molecule are shown as sticks. (B) Close up representation of the HM/PIF-pocket. Alpha helices lining the pocket (α-B and α-C) are indicated. (C,D) Structure of compound 1, and scaffold indicating positions of R1, R2 and R3. (E,F) Scheme of AGC protein kinase activation by phosphorylation (E) and by small compounds (F). The red circle represents protein phosphorylation while the green one represents a small compound. The represented activation of the kinase involves conformational changes in the HM/PIF-pocket and in the ATP binding site, as described for PKB and MSK.

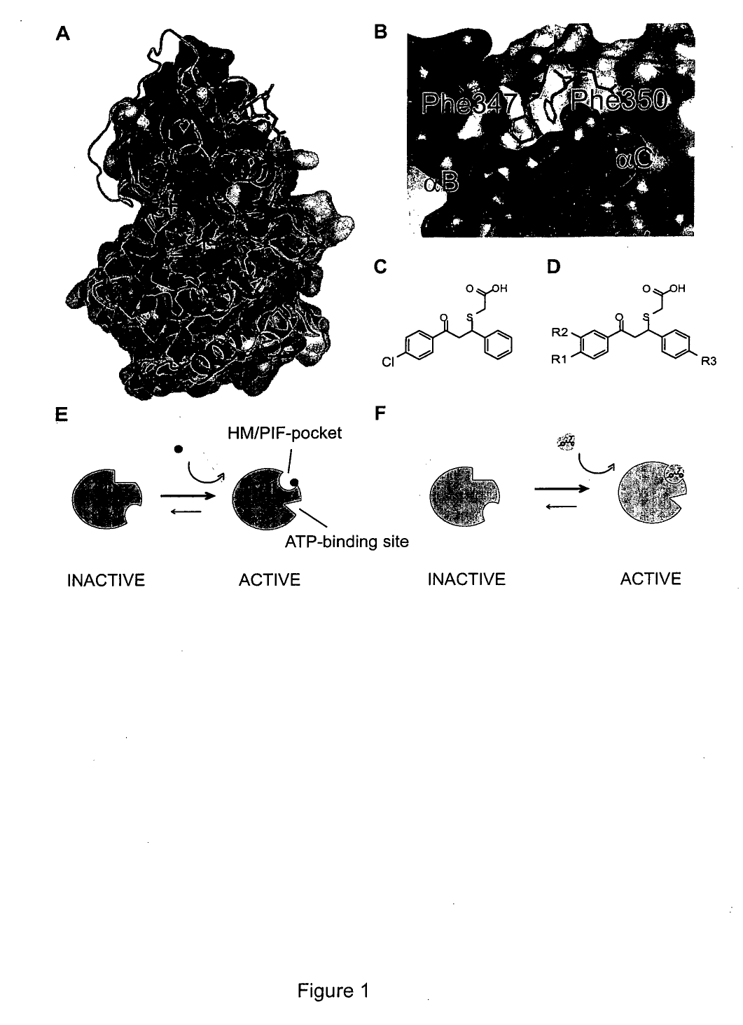

FIG. 2—Activation of PDK1 and mutants by phosphopeptides and small compounds designed to target the HM/PIF-pocket. The intrinsic activity of purified PDK1 and the indicated mutants were measured in vitro using the polypeptide T308tide as a substrate in the presence or absence of the indicted P-HM-polypeptides, PIFtide or small compounds. (A) Effect of phosphopeptides (closed), dephosphopeptides (open), derived from the HM of PKB (squares) or RSK (circles) on the specific activity of PDK1. (B) Effect of compound 1-comprising a carboxyl group- (open triangles) and its methyl-ester form, compound 2 (closed triangles) on PDK1 activity. (C) Effect of P-HM-RSK and compound 1 (both at 20 μM) on PDK1 wild type (WT) and PIF-pocket PDK1 mutants [Gln150Ala], [Thr148Val], [Leu155Glu] and [Ile119Ala]. (D) Compound 1 activated PDK1 wt (open triangles) but not PDK1[Val127Leu] (open rhombi). (E) PDK1[Val127Leu] retained the ability to be activated by PIFtide with similar AC₅₀. (F) Arg131, which forms part of the PDK1 P-HM binding site adjacent to the HM/PIF-pocket, is required for the activation by PIFtide and compound 1. (G) The activity of a PDK1 50-360[CT-PIF] chimera is not affected by PIFtide or compound 1. PDK1 50-360[CT-PIF] comprises the catalytic domain of PDK1 joined to the C-terminal residues from protein kinase PRK2, comprising the PDK1 interacting fragment (PIF).

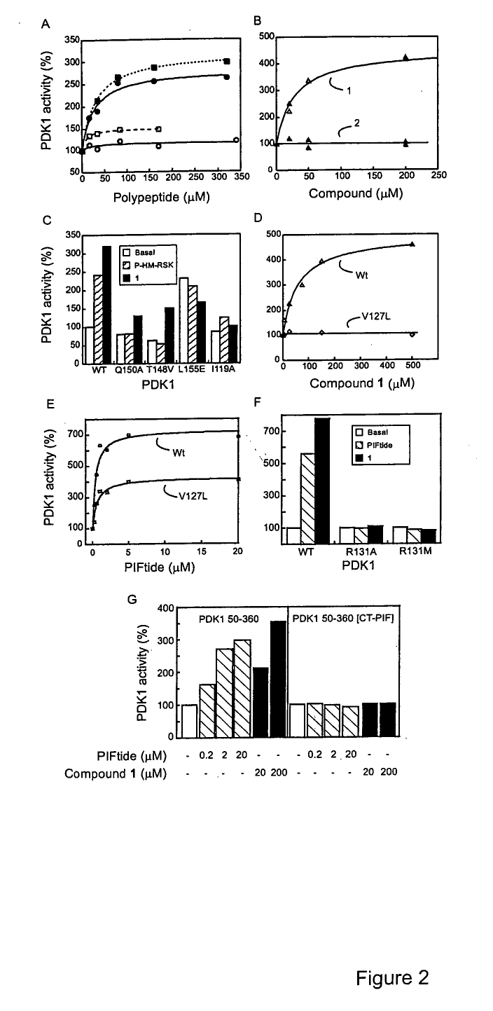

FIG. 3—Small compounds displace the binding of PDK1 1-556 to the HM peptide derived from its substrate PRK2, PIFtide. Surface plasmon resonance measurements were carried out on a BiaCore system to measure the interaction between GST-PDK1 and biotin-PIFtide coupled to a streptavidin-coated chip. (A) Direct measurement of PDK1 interaction with biotin-PIFtide. PDK1 was injected at the indicated concentrations (nM). For the following competition experiments, PDK1 45 nM was used. (B) PDK1 interaction with biotin-PIFtide was abolished by pre-incubation with PIFtide. (C) Compound 1 (open triangles) displaced the interaction of PDK1 with biotin-PIFtide while compound 2 (closed triangles), which lacks the carboxylate group, did not affect the binding of PDK1 to the chip. The data points represent the maximal response units obtained after 30 s injection in the presence of the indicated concentration of compound. In (A) and (B) the spikes at the start and end of injection have been eliminated for clarity.

FIG. 4—Small compounds designed to bind the HM/PIF-pocket of PDK1 can block the PDK1-dependent phosphorylation and activation of substrates that require docking to the HM/PIF-pocket of PDK1. (A) Effect of compound 1 (100 and 200 μM) on PDK1 phosphorylation of its substrate S6K1. The phosphorylation reaction was performed in vitro with [γ³²P]ATP and then separated on SDS-PAGE followed by exposure to phosphorimager. The radioactive band corresponding to phosphorylated GST-56K1-T2[Thr412Glu] (S6K[412E]) is indicated. (B) PDK1 or PDK1[Val127Leu] (PDK1[V127L]) were pre-incubated with GST-□N-SGK1 [Ser422Asp] (SGK[422D]) in the presence or absence of compound 1 (60 μM) followed by the addition of SGK substrate to measure its activity. (C and D) Effect of compound 1 on the activation loop phosphorylation and activity of S6K mutants transfected into HEK293 cells. Cells overexpressing GST-S6K1-T2[Thr412Glu] and the inactive mutant S6K1-T2[Thr412Ala] (S6K[412E]) were treated for 90 minutes with 200 μM compound 1 or vehicle (DMSO), followed by cell lysis. (C) The activity of S6K was measured in vitro after glutathione sepharose purification; aliquots of purified proteins were separated on SDS-PAGE and the level of activation loop phosphorylation was visualized by immunoblotting using phospho-specific antibodies that recognize the phosphorylated activation loop, followed by stripping of the membranes and re-probing with anti-GST to verify the presence of equal amounts of protein. (D) Cells overexpressing GST-56K1-T2[Thr412Glu] were treated as in (C) but S6K activity was measured in vitro after immunoprecipitation of the GST-fusion protein with anti-GST antibodies; the level of S6K expression was analysed by western-blotting of crude extracts. In (C) and (D), the duplicates are from independent transfections performed in parallel.

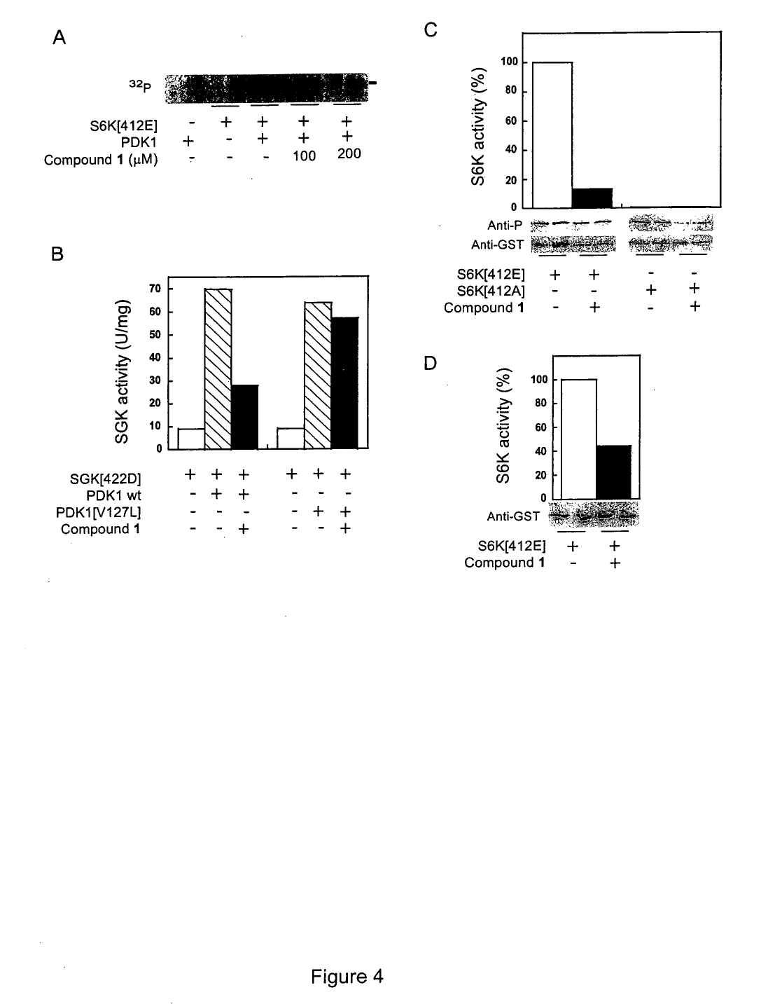

FIG. 5—Modulation of PDK1 conformation by phosphopeptides and small compounds. Effect of P-HM-polypeptides and compound 1 on the emission fluorescence of trinitrophenyl-ATP (TNP-ATP)/PDK1. The fluorescence emission spectra from TNP-ATP were recorded in the presence or absence of P-HM-polypeptides, PIFtide and compound 1. (A) Effect of P-HM-RSK (65 μM) on the fluorescence by TNP-ATP. (B) Effect of compound 1 on the fluorescence emission from TNP-ATP. Fluorescence intensities are expressed as %. The maximal effect of PIFtide in this assay (65%) was obtained at 10 times the value of its Kd for PDK1. The effect of P-HM-PKB was identical to that by P-HM-RSK.

FIG. 6—A mutation at Thr226 in PDK1 uncouples the ligand binding to the HM/PIF-pocket from activation of the kinase. The catalytic domain (CD) of PDK1 50-360 and its mutants Phe224Trp and Thr226Trp were expressed in insect cells and purified. (A) PDK1 activity was measured in the presence or absence of the HM-polypeptide PIFtide or compound 1 using T308tide as a substrate. PDK1 CD wt and PDK1 [Phe224Trp] were activated by PIFtide and compound 1 whereas PDK1 CD [Thr226Trp] mutant was not activated. (B,C,D,E) PDK1 [Phe224Trp] and PDK1 [Thr226Trp] retain significant binding to P-HM polypeptides and PIFtide. Surface plasmon resonance measurements were carried out on a BiaCore system to evaluate the binding affinities of PDK1 CD wt, PDK1 [Phe224Trp] and PDK1 [Thr226Trp] to HM-polypeptides. Biotin-P-HM-polypeptide derived from S6K1 was immobilized on a streptavidin-coated sensor chip and the specific interaction with PDK1 was recorded. (B) The direct binding of PDK1 CD wt to biotin-P-HM-S6K was recorded at the indicated concentrations of PDK1 CD. The Kd (0.05 μM) was estimated based on the response units obtained at each concentration at equilibrium (insert). (C,D,E) PDK1 CD (50-360), wt (0.35 μM), PDK1 [Phe224Trp] or PDK1 [Thr226Trp] were injected onto the system alone or in the presence of the indicated concentrations of PIFtide.

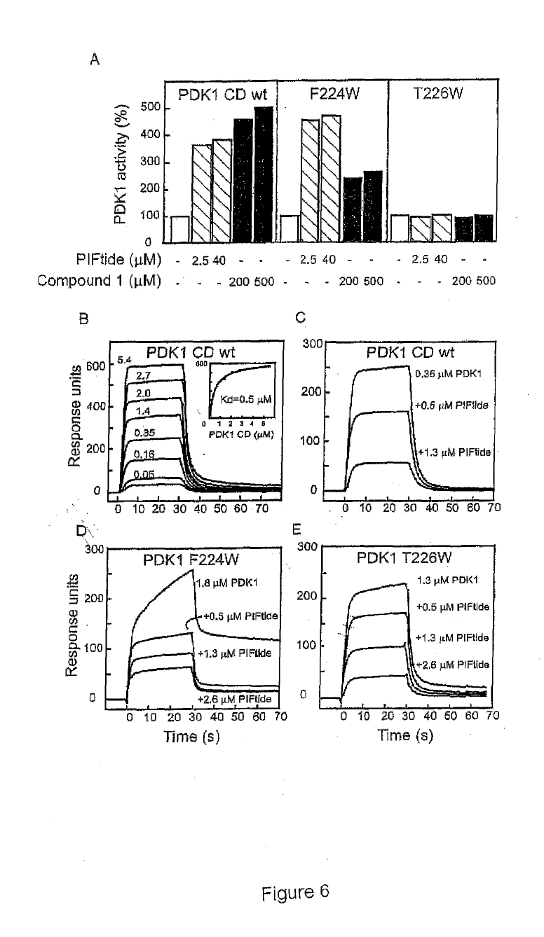

FIG. 7—Model for compound 1 docking and activation of PDK1. (A) Model for compound 1 docking to the HM/PIF-binding pocket on PDK1. Molecular docking of compound 1 onto PDK1 crystal structure was unreliable. Therefore, the presented model was built based on the mode of interaction of the C-terminal Phe347 and Phe350 to PKA, whereas the position of the carboxylate was further adapted to fit the biochemical results which suggest a specific interaction with the positive charge of Arg131. For this model, we have moved Phe157 out of the second Phe cavity by choosing another rotamer of Phe157. (B) Scheme for the activation of PDK1 by compound 1. The mechanism of activation of PDK1 by compound 1 involves the binding to the HM/PIF-pocket. We here describe Val127, Ile119, Leu155, and Arg131 as residues which are important for the activation of PDK1 by compound 1. Val127, Ile119 and Leu155 are expected to provide hydrophobic interactions to compound 1, while Arg131 appears to be responsible for the interaction with the carboxylate from compound 1. The activation process also requires the presence of the phosphate at the activation loop (Komander et al., 2005). As the mechanism of activation of AGC kinases appears highly conserved, the model for the activation of PDK1 by small compounds may be the starting point for the development of small compounds targeting AGC kinases by similar approaches.

FIG. 8—Characterization of compound 1 interaction with PDK1 50-360 by Isothermal Titration Calorimetry (ITC). Upper panel, raw data of the titration of compound 1 into PDK1 50-360. Lower panel, integrated heats of injection, corrected for the heat of dilution, with the solid line corresponding to the best fit of the data using Origin™ software. The best fit corresponded to a model with one type of sites. The parameters defining the fitted curve are K_{d}=18+2 μM, ΔH=−2.1+0.1 kcal·mol⁻¹, TΔS=4.3+0.2 kcal·mol⁻¹, and N=1.0+0.1. The calorimetric titration was performed using the VP-ITC instrument from MicroCal Inc. (Northampton, Mass.) as previously described (Schaeffer et al., 2002) with the following modifications. PDK1 and compound 1 were prepared in 50 mM Tris-HCl (pH 7.5), 175 mM NaCl, 1 mM DTT and 3% DMSO. Titration was performed by 30 successive injections (10 μl) of compound 1 (0.9 mM) into a 1.41 ml reaction cell containing PDK1 50-360 (500 M). Raw calorimetric data were corrected for the heat of dilution, and analyzed using the program Origin™ provided by the manufacturer. Binding stoichiometry, enthalpy and equilibrium dissociation constants were determined by fitting corrected data to a model with a single binding site.

FIG. 9. Molecular modelling suggests a widely conserved binding site for the tail/linker phosphoSer/Thr within the catalytic domain of AGC kinases. (A) Model of active PKBβ shown as a ribbon representation with side chains of selected residues. The kinase domain is shown in green and the tail/linker in red. Phosphate groups are shown in yellow. K/R residues predicted to bind the phosphate of phosphoT451 are shown in blue. Other phosphate-binding residues are in cyan. The hydrophobic motif, glycine-rich loop and ATP analogue are shown in magenta, orange and white, respectively. Partial sequence alignment of the tail/linker (B) and predicted binding site for the tail/linker phosphate (C) of AGC kinases. (B, C) Phosphorylation sites/phosphate-mimicking residues are shown in red. The aromatic residues that define the hydrophobic motif are underlined. Basic residues predicted to bind the tail/linker phosphoSer/Thr are shown in blue and labelled 1 to 4. Sequences are human except S6K1 (rat, p70 isoform) and RSK2 (mouse).

FIG. 10. Role of the tail phosphorylation site in activation and phosphorylation of AGC kinases in vivo. COS7 cells were transfected with plasmid expressing HA- or GST-tagged wt or mutant kinase. After 16 h and a final 4 h serum-starvation period, cells were exposed to 1 μM insulin for 10 min (PKBa), to 20 nM EGF for 30 min (S6K1) or 15 min (RSK2), to 10 μg/ml anisomycin for 40 min (MSK1) or left in serum-containing medium (PRK2_{Δ1-500}) and then lysed. The kinases were precipitated from aliquots of the cell lysates with antibody to the HA tag or with glutathione beads. The precipitates were subjected to kinase assay, to immunoblotting with the indicated phosphorylation site-specific Ab or anti-HA Ab or stained for protein. Experiments were repeated at least 3 times and activity data (expressed as percent) are means±SD.

FIG. 11. The tail phosphate-binding site is essential for normal activation and phosphorylation of AGC kinases in vivo. The activity and phosphorylation state of wt and mutant AGC kinases expressed in COS7 cells (A, C) or S2 cells (B) were analyzed as described in the legend to FIG. 10, except that Drosophila S6K was activated by exposure of cells to 1 μM insulin and 10 μM pervanadate for 15 min. Experiments were repeated at least 3 times and activity data (expressed as percent) are means±SD.

FIG. 12. Further evidence of the tail phosphate-binding site. The activity of wt and mutant PRK2_{Δ1-500} (A) or MSK1 (B) expressed in COS7 cells were analyzed as described in the legend to FIG. 10. However, in (A) kinase activity was also determined in the presence of the indicated concentration of Zn²⁺ and expressed as percent of activity in the absence of Zn²⁺. Experiments were repeated at least 3 times and activity data are means±SD.

FIG. 13. The tail phosphate synergistically enhances the ability of the HM phosphate to activate S6K, dependent on the tail phosphate-binding site. (A) The kinase activity of purified kinase domain of S6K1 (S6K1₁₋₃₆₄), pre-phosphorylated by PDK1 in the activation loop, was determined in the presence of increasing concentrations of synthetic S6K1 tail peptide (residues 366-395) that was either non-phosphorylated (S371/T389), phosphorylated at Ser371 (pS371/T389) or Thr389 (5371/pT389) or phosphorylated at both sites (pS371/pT389). (B) The kinase activity of S6K1₁₋₃₆₄ and S6K1₁₋₃₆₄K144N, either pre-phosphorylated or not by PDK1, was determined in the absence or presence of 90 μM S371/pT389 or pS371/pT389. (C) pS371/pT389 S6K1 tail peptide was biotinylated and used to coat streptavidin Sensor Chips. The chips were thereafter analyzed for binding to purified GST-S6K1₁₋₃₆₅ or GST-56K1₁₋₃₆₅K144N. (A-C) Experiments were repeated at least 3 times and activity data (expressed as percent) are means±SD.

FIG. 14. The tail phosphate promotes a compact global conformation of the AGC kinase domain and protects the tail phosphate-binding site and αC-helix from solvent exposure.

(A) Effect of S6K1 tail peptides, described in the legend to FIG. 13, on global deuteron uptake by purified GST-56K1₁₋₃₆₅. (B) Kinase activity of wt and mutant GST-PKCζ. (C) Global deuteron uptake by wt and mutant GST-PKCζ. (D) Local deuteron uptake by wt and mutant GST-PKCζ. In the PKCζ model, peptides showing strong and no-or-slight protection by the tail phosphate are shown in pink and grey, respectively. The panels show HX curves of the peptides. Experiments were repeated 2 times (A, C, D) or 3 times (B) and data are means±range (A, C) or ±SD (B)

FIG. 15. The tail phosphate may correspond to E333 of PKA. (A) Analogous positions and interactions of E333 of PKA and the tail phosphate. Left and right panel shows a structure of PKA (1ATP) and our model of RSK2, respectively. Colour codes are described in the legend to FIG. 9A, except that residues known/thought to interact with the turn motif phosphate of PKA and phosphoS375 of RSK2 are shown in grey. (B) Effect of mutation of E332/E333 and R56 on autophosphorylation and activity of PKAa expressed in E. coli. (C) Effect of mutation of S375 in RSK2 expressed and analyzed as described in the legend to FIG. 10. (B and C) Data are means±SD of 3 independent experiments. (D) Proposed alignment and naming of phosphorylation sites in the tail of AGC kinases.

FIG. 16. Growth factor-activated AGC kinases contain three conserved phosphorylation sites required for full activation. The growth factor-activated AGC kinases PKB, S6K, RSK, MSK, PRK and PKC contain three phosphorylation sites required for full activation: the activation loop site, the tail/linker site and the hydrophobic motif site (numbered according to the kinases used in the present study). PRK2 and some PKC family members, including PKCζ, contain a phosphate-mimicking Asp or Glu residue at the hydrophobic motif site. PKA also has a phosphorylation site, named the turn motif site, in the tail region. The kinases thought to phosphorylate the conserved phosphorylation sites in the various AGC kinases are shown in the brackets.

FIG. 17. Conservation of the predicted binding site for the tail phosphoSer/Thr in 26 human growth factor-activated AGC kinases and in 4 orthologs of S6K. Partial amino acid sequence alignment of the kinase domain (A) and tail region (B) of various AGC kinases. Conserved basic residues that may bind the tail phosphoSer/Thr are shown in blue and labelled basic residue 1 to 4. Phosphorylation sites/phosphate-mimicking acidic residues are shown in red. The aromatic residues that define the HM are underlined. The sequence is human if nothing else is indicated.

FIG. 18. Molecular modelling of S6K1 and RSK2 suggests a homologous binding site for the tail phosphoSer/Thr within the small lobe of the kinase domain. The kinase domain and linker of S6K1 (A) and RSK2 (B) were modelled as described in Materials and Methods. Colour codes are as described in the legend to FIG. 9A.

FIG. 19. Effect of motation of the tail phosphorylation site to a glutamic acid residue. The activity and phosphorylation state of wt and mutant PKBα, RSK2, MSK1, and PRK2Δ1-500 expressed in COS7 cells were analyzed as described in the legend to FIG. 2. Experiments were repeated at least 3 times and activity data (expressed as percent) are means+/−SD.

FIG. 20. Effect of various mutations in the tail phosphate-binding site on the activity and phosphorylation state of AGC kinases. The activity and phosphorylation state of wt and mutant S6K1 (A), MSK1 (B), PRK2Δ1-500 (C), or ΔPH-PKB<−S473E (D, E), expressed in COS7 cells were analyzed as described in the legend to FIG. 10. Experiments were repeated at least 3 times and activity data (expressed as percent) are means+/−SD.

FIG. 21 (A) Aliquots of S6K1 protein from bar 1, 2, 5 and 6 in FIG. 5B were subjected to immunoblotting with phosphorylation site specific Ab against the activation loop or anti-HA Ab. (B) The kinase activity of PKB<143-479, prephosphorylated by PDK1 in the activation loop, was determined in the presence of increasing concentrations of synthetic PRK2 tail peptides encompassing the HM, which were either nonphosphorylated (PIFtide) or phosphorylated at the tail site (pT958-PIFtide). The figure shows a representative experiment with kinase activity expressed as percent. (C) The kinase activity of purified PDK1 was determined in the presence of increasing concentrations of synthetic S6K tail peptide (residues 366-395) that was either phosphorylated at T389 (S371/pT389) or phosphorylated at both 5389 and 5371 (pS371/pT389). Kinase activity is expressed as percent, and data are means+/−SD of 3 independent experiments. (D) Synthetic S371/pT389 S6K tail peptide and pS371/pT389 S6K tail peptide were biotinylated and used to coat streptavidin Sensor Chips. PDK150-360 was injected at different concentrations (0.05-5.4 mM) onto the peptide coated chips. In the inserts, the kinetic constants were obtained by fitting the data to a hyperbola using kaleidagraph software. Representative results from one of several experiments are shown.

FIG. 22. Effect of mutation of the tail site and the tail phosphate-binding site on the kinase activity of PKCζ. COS7 cells were transfected with plasmid expressing GST-tagged wt or mutant PKCζ. After 20 h the cells were lysed, and the kinases were precipitated from aliquots of the cell lysates with glutathione beads. The precipitates were subjected to kinase assay or stained for protein. Experiments were repeated at least 3 times and activity data (expressed as percent) are means+/−SD.

FIG. 23. Amide hydrogen (1H/2H) exchange of PKCζ monitored by mass spectrometry. (A) Global exchange analysis of wt PKCζ. Deconvoluted spectra were obtained after 0, 1, 5, 15, and 45 min deuteration. The insert depicts the charge-state distribution of the intact non-deuterated protein. (B) Deuterium incorporation vs. exchange time for wt PKCζ. (C) Local exchange analysis by pepsin digestion of labelled wt PKCζ and PKCζT560A tail site mutant. Mass spectra are derived from peptide I294-L313. (D) Deuterium incorporation of peptide I294-L313 vs. exchange time.

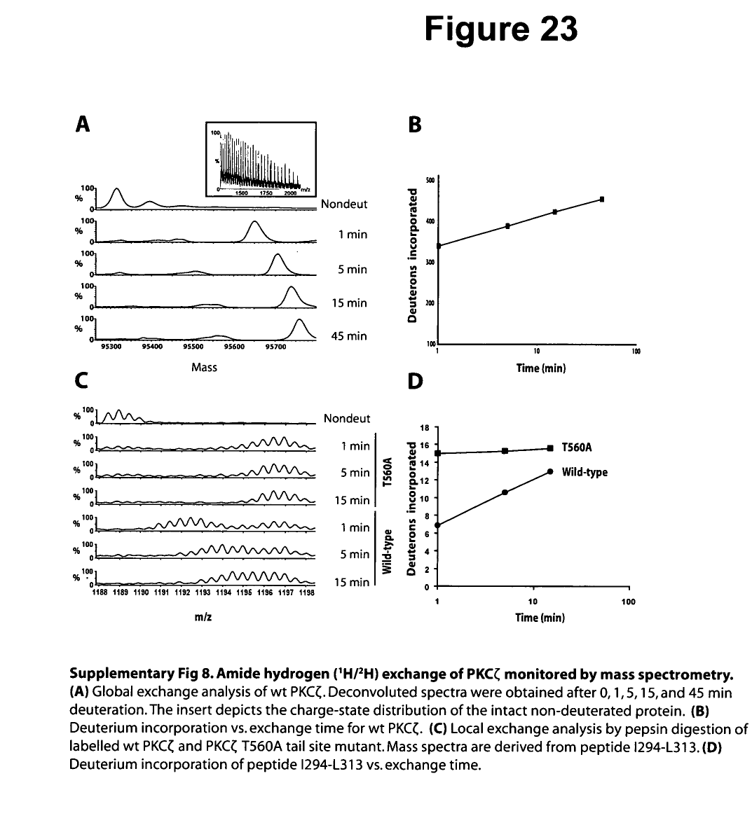

### DETAILED DESCRIPTION OF THE INVENTION

Previous work suggested a molecular model to explain the molecular mechanism of activation of AGC kinases by HM phosphorylation. We concluded that the essence of the mechanism involves a ligand binding to the regulatory site, and therefore we here further studied the allosteric mechanism of regulation and explored if the essential aspects of the regulation could be mimicked by small molecules, which, in the future, could be developed into orally available drugs. Based on the molecular model of activation of AGC kinases and in silico analysis of the HM/PIF-pocket structure, we have now developed small molecular weight compounds which activate PDK1. Mutagenesis at the centre of the HM/PIF-pocket (e.g. Leu155Glu and Val127Leu) blocked the effect of the compounds on PDK1, suggesting that the HM/PIF-pocket is the target site for compounds according to general formula 1, especially compound 1. The ability of compounds to activate PDK1 also required the positive charge from Arg131, which forms part of the associated phosphate binding site that physiologically interacts with the phosphate within the HM phosphorylation site from substrates (Collins et al., 2005). The activation by compound 1 required the presence of the free carboxylate group, since compound 2, possessing a methyl-ester instead, lost the ability to bind and activate PDK1. These results supported the notion that compound 1 interacted with the HM/PIF-pocket and with the positive charge from Arg131 residue which forms the adjacent phosphate binding site. This was further supported by surface plasmon resonance experiments which showed that the small compounds can displace the binding between PDK1 and the HM polypeptide PIFtide. In further agreement with this notion, compound 1 did not affect the activity of a PDK1 chimera which has the sequence of PDK1 linked to the C-terminal 48 residues from PRK2 that includes the 24 aminoacids present in PIFtide. In addition, the compounds were able to block PDK1 phosphorylation of its substrates S6K and SGK, which depend on the docking to the HM/PIF-pocket of PDK1 for efficient phosphorylation and activation. Therefore, we conclude that the ability of compound 1 to activate PDK1 is related to its ability to interact specifically with the HM/PIF-pocket and the associated phosphate binding site of PDK1. On the other hand, the specificity of the effect of compounds on PDK1 was verified by synthesizing compound analogues. Some of these analogues where inactive and unable to interact with PDK1; such marked structure-activity relationship argues for selectivity of compounds and helps to define the requirements to activate PDK1. Finally, we probed the ATP binding site conformational change induced by P-HM peptides in solution and verified that the effect of compound 1 was identical to the effect induced by P-HM polypeptides on PDK1, suggesting that they both brought about a similar conformational change, which effects on the ATP binding site. Altogether our results provide extensive evidence that small compounds can modulate the conformational inactive-active transition of PDK1 by targeting the HM/PIF-pocket regulatory site. To our knowledge, this is the first report of a rational development of small compounds that mimic a conformational transition physiologically induced by phosphorylation.

Our work was based on the matured model that described the molecular mechanism of activation by HM phosphorylation in AGC kinases (Biondi, 2004; Newton, 2003b; Pearl and Barford, 2002). The actual success in the development of these compounds strengthens the validity of the models used as a base of our hypothesis and further highlights the HM/PIF-pocket as a minimum requirement for the modulation of activity. In a general AGC kinase, the HM phosphorylation triggers the intra-molecular binding of the P-HM to the HM/PIF-pocket and the contiguous phosphate binding site. In the specific case of PDK1, it binds the HM of its substrates inter-molecularly in a phosphorylation dependent manner. Thus, the conformational changes induced by phosphorylation of AGC kinases can be simplified to a system where a ligand binds to the HM/PIF-pocket regulatory site in a phosphorylation-dependent manner.

Analogous mechanisms of intra-molecular and inter-molecular regulation of protein kinases have been described. We have found that the mechanism of regulation of GSK3 by N-terminal phosphorylation involves the intra-molecular binding of the N-terminal phosphorylated residues to a phosphate binding site in the catalytic domain, implying a phosphorylation-dependent intra-molecular docking (Dajani et al., 2001; Frame et al., 2001), which resembles the regulatory C-terminal HM phosphorylation in AGC kinases (Yang et al., 2002). In striking similarity with PDK1, the regulatory phosphate binding site in the catalytic domain of GSK3 is required for the inter-molecular binding of substrates in a phosphorylation-dependent manner (Frame et al., 2001). Another example is the mechanism of inhibition of Tyr kinases by C-terminal tail Tyr phosphorylation. In this case, the C-terminal Tyr phosphorylation prompts the intra-molecular binding to the Src homology (SH2) domain (Sicheri et al., 1997; Xu et al., 1997). This binding triggers the docking of a Trp residue from the linker region into the small lobe of the catalytic domain, which then stabilizes an inactive conformation (Gonfloni et al., 1997). Interestingly, the site of binding of the Trp residue is homologous to the HM/PIF-pocket site in AGC kinases. Thus, phosphorylation-dependent intra- and inter-molecular interactions, in their different forms, are physiologically relevant in diverse protein kinases and may be widely present in nature in other proteins. Our success in the generation of small molecules that can activate PDK1 by mimicking the phosphorylation-dependent docking of substrates suggests that it may be possible to modulate phosphorylation-dependent conformational changes with small compounds in other systems.

The structure of the catalytic domain in active protein kinases is extremely conserved. In contrast, the inactive structures appear to be diverse. Thus, in the crystal structures of inactive PKB and MSK, the alpha-C helix is disturbed and the DFG motif is distinctly positioned as in the active structures (Smith et al., 2004; Yang et al., 2002). Thus, activation of PKB and MSK is thought to involve a large conformational transition. On the other hand, the crystal structure of the inactive PDK1 protein mutated in the activation loop phosphorylation site (Ser241Ala) has the αC-helix and the DFG motif in a similar position as in the active form (Komander et al., 2005). The authors concluded that the structure of PDK1 Ser241Ala represents the inactive structure of PDK1 and therefore the inactive-active transition may not involve similar conformational changes as thought for PKB or MSK. Since a strong electron density was observed at the position equivalent of the phosphor-Ser241 in PDK1 [Ser141Ala] crystal, it is also possible that this unidentified high affinity binding ligand stabilized the active conformation of the αC-helix and the DFG motif in those crystals.

We have here attempted to probe the phosphorylation-dependent conformational change of PDK1 in solution by using a fluorescent analogue of ATP, TNP-ATP. In fact, PIFtide and P-HM-polypeptides prompted a decrease in TNP-ATP fluorescence intensity, suggesting a significant conformational change in the ATP binding site. We further provide evidence that the conformational change is prompted allosterically by the binding to the HM/PIF-pocket, since small compounds that bind to this pocket are able to mimic the same effects on TNP-ATP. Our results therefore show that the small compounds prompted equivalent conformational changes to PIFtide or phosphor-peptides.

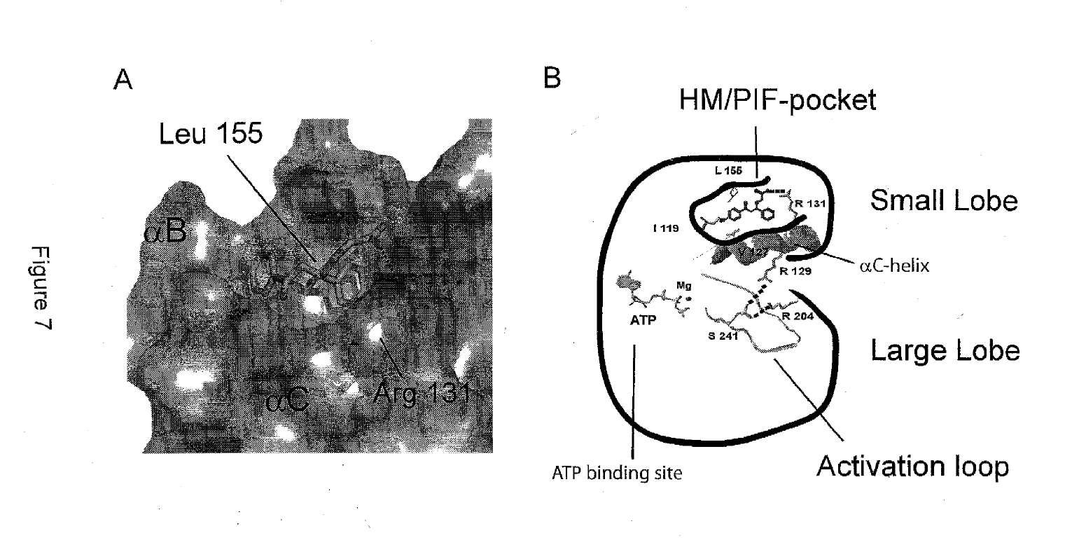

Our mutagenesis work further defines, at a molecular level, the regions of PDK1 that are required for the activation. Thus, a small molecule occupying a limited space in the HM/PIF-pocket defined by Ile119, Leu155, Val127 and Arg131, was able to activate PDK1. Mutations in these residues blocked activation by compound 1. It appears likely that the hydrophobic residues Ile119, Leu155 and Val127 form part of the hydrophobic pocket required for binding the phenyl groups of compound 1 while the positive charge on Arg131 may make specific electrostatic interactions with the carboxylate group from compound 1. This is in agreement with in silico docking experiments. Mutations in Ile119, Leu155 and Arg131 also inhibited PIFtide and P-HM-polypeptide binding and activation of PDK1 (Biondi et al., 2000; Biondi et al., 2002). By contrast, other residues that were important for binding the HM-polypeptide were found not to be essential for the activation process since they were not required for activation by compound 1. This was the case for Gln150 and Lys115, which play a major role in PDK1 binding to PIFtide (Biondi et al., 2000; Komander et al., 2005). In addition, we here described Thr226 as an unexpected determinant in the allosteric effects induced by HM-polypeptides. The equivalent residue in other AGC kinases is Phe, Leu or Met, while it is Trp in Aurora protein kinase, which is most closely related to AGC kinases and may share an analogous mode of regulation by interaction with TPX2 (Bayliss et al., 2003). Therefore, we predicted that the mutation of Thr226 to Trp may not have major detrimental effects on protein folding and stability. In agreement with this, PDK1 [Thr226Trp] had only marginally lower basal activity. Nevertheless, in spite of binding PIFtide with high affinity, PDK1 [Thr226Trp] was not activated by PIFtide or compound 1. Thr226 is located in the sequence DFGT, just next to the DFG motif. We can speculate that the molecular impediments in the activation process of PDK1 [Thr226Trp] are related to the contiguous DFG motif. Such model would be in agreement with the important role ascribed to this motif in the activation process of PKB (Yang et al., 2002). Komander et al. had characterized the requirement of the activation loop for PDK1 activation (Komander et al., 2005). Taken together, our present model suggests that the molecular mechanism of activation involves the docking of a ligand to the HM/PIF-pocket, as defined for compound 1, activation loop phosphorylation, and, finally, it also requires the integrity of the region surrounding the DFG motif in PDK1 which is disrupted by Thr226Trp mutation (FIG. 7).

Although the small compounds were able to mimic the conformational changes induced by phosphorylation of the HM from substrates, the small compounds were not able to mimic all the effects that the HM polypeptides exert on PDK1. Thus, while PDK1 stability to temperature is increased by 10° C. in the presence of PIFtide (Biondi et al., 2000), compound 1 did not stabilize PDK1 to thermal denaturation (data not shown), suggesting that the larger set of interactions by PIFtide may be required for this increased stability. Importantly, we here demonstrate that all additional specific interactions set by physiological docking polypeptides outside the HM/PIF-pocket are not required for the activation of PDK1.

We predict that the HM/PIF-pocket regulatory site in other AGC kinases may also be targeted by small compounds which could stabilize the inactive structures and inhibit the intrinsic activity of the enzymes. Interestingly, compound 1 and compound 3 (Table II), which stimulated PDK1 intrinsic activity, inhibited PKCζ in our panel. In contrast, compounds 4 and 5, which possess single differences in the identity of substituents in R1 and R3, did not inhibit PKCζ in a comparable manner. It is thus possible that compounds 1 and 3 are allosteric inhibitors of PKCζ which target the HM/PIF-pocket site.

Further object of the invention is therefore a method of inhibiting PKCζ by small molecules.

Finally, compound 1 proved to be reasonably specific for PDK1 and did not affect significantly the activity of other AGC kinases. We here identified that mutation in two non conserved residues, Ile119 and Val127, abolished activation by compound 1. In other AGC kinases, the equivalent residue to Val127 is Thr, Ile, Leu or Ala and the equivalent to Ile119 is Val, Leu, Asn or His. Thus, our initial characterization of the compound 1 binding site suggests that the pocket may be amenable to the development of specific compounds directed to other AGC kinases.

In conclusion, the rational development of compound 1 proves that small molecule compounds can provide all the necessary requirements to induce the conformational changes required for activation of AGC protein kinases, by interacting with the regulatory HM/PIF-pocket. The activation induced by the small molecular weight compounds described here is physiologically achieved in AGC kinases by HM phosphorylation. The general mechanism by which the phosphorylation transduces into a conformational change in AGC kinases is a phosphorylation-dependent docking interaction of a regulatory sequence to the catalytic domain. A similar mechanism operates in other well characterized examples of protein regulation by phosphorylation. Therefore, our work provides the first evidence that, through understanding the molecular mechanism by which a protein is regulated via phosphorylation, it is possible to rationally design small molecular weight drugs to mimic the conformational states physiologically achieved by phosphorylation. Such compounds could have future applications in the treatment of disease by specifically modulating the phosphorylation-dependent conformation of a target protein.

### Methods

General materials and methods, the expression and purification of protein kinases, the protein kinase activity tests, in silico screening and the synthesis and characterization of compounds is described in the Supplementary information section.

### Peptides

Polypeptides T308tide: KTFCGTPEYLAPEVRR; Crosstide: GRPRTSSFAEG; Kemptide: LRRASLG; HM-PIFtide: GFRDFDY and PIFtide: REPRILSEEEQEMFRDFDYIADWC, were synthesised by us and the purity verified by HPLC. Where required, the polypeptides were HPLC purified. Polypeptides used were at least 75% pure; the identity of polypeptides was confirmed by N-terminal sequencing and mass spectrometry. P-HM-PKB: KGAGGGGFPQFS(P)YSA, HM-PKB: KGAGGGGFPQFSYSA, P-HM-RSK: KGAGGGGFRGFS(P)FVA and HM-RSK: KGAGGGGFRGFSFVA, were obtained from ThermoHybaid, and identity verified by mass spectrometry. Biotinylation of PIFtide was performed by amine-coupling using Biotin-N-hydroxysuccinimide (Perbio) following the instructions provided by the manufacturer. Modified peptides were re-purified to over 90% purity on a Jupiter C5 HPLC column (Phenomenex) applying a linear gradient from 17% acetonitrile/0.1% trifluoroacetic acid/83% water to 70% acetonitrile/0.1% trifluoroacetic acid/30% water, lyophilised and kept at −20° C. until use. Biotin-P-HM-S6K (Biotin-KQTPVDS(P)PDDSTLSESANQVFLGFT(P)YVAPSV was a gift from Morten Frodin (Copenhagen, Denmark).

Interaction of PDK1 with PIFtide and the Displacement by Small Compounds.

Binding of PDK1 to PIFtide was analysed by surface plasmon resonance on a BiaCore 3000 system using a streptavidin-coated Sensor chip (SA) and biotin-PIFtide, as previously described (Biondi et al., 2001). Biotin-PIFtide was bound to the chip to a level of 15 to 25 response units in different experiments. GST-PDK1 bound to biotin-PIFtide with an affinity of 90 nM, in good agreement with previous data obtained using the BiaCore system. For interaction-displacement assays, GST-PDK1 (60 nM) was injected (30 μl/min) in a buffer containing 10 mM HEPES pH 7.5, 150 mM NaCl, 0.005% Tween20, 1 mM DTT and 1% DMSO, in the presence or absence of compounds. GST-PDK1 was pre-incubated with compounds for 1 to 10 minutes before injection into the system, with similar results. Experiments were performed at least twice using different biotin-PIFtide coated chips, with similar results obtained on each occasion.

### Probing the Conformation of the ATP Binding Site in PDK1.

The activation of PDK1 by P-HM-polypeptides and small compounds is due to a change in the conformation of the enzyme. We probed the conformation of the ATP binding site in PDK1 by scanning the steady-state fluorescence of TNP-ATP/PDK1, in the presence or absence of P-HM-polypeptides, PIFtide, or small molecular weight compounds. Data were obtained in a Varian Cary Eclipse spectrofluorometer (excitation γ=479 nm; emission scanning, γ=500-600 nm; excitation slit=10 nm; emission slit=10 nm) at a rate of 200 nm/min, with 150 datapoints/100 nm scanning and 0.3 s averaging time. The incubation was performed at 20° C. in a buffer containing 50 mM Tris-HCl pH 7.5, 0.1 mM EDTA, 187 mM NaCl, 40 μM TNP-ATP, 15 μM PDK1 catalytic domain (50-360), 1 mM DTT and 1% DMSO. Non-phosphorylated HM-polypeptides increased the fluorescence intensity of TNP-ATP in the absence of PDK1 and were not used in the study. In preliminary tests, inclusion of 2.5 mM MgCl₂ in the assay mix produced similar results. Data for each condition are the average of 3 scans.

### Supplementary Materials and Methods

### General Materials and Methods

Complete protease inhibitor cocktail tablets were from Roche. Protein concentration was estimated using a Coomassie reagent from Perbio. Protein was concentrated using Vivaspin concentrators (Vivascience). Glutathione sepharose, Ni-NTA sepharose and chromatography columns were from Amersham Pharmacia Biotech. A phosphor-specific antibody which recognizes the phosphorylated activation loop of several AGC kinases was from Upstate Biotechnology. Anti-GST (B-14) was from Santa Cruz Biotechnology. Chemiluminescent substrate used in western-blot (Roti-lumin) was from Roth. Western-blot stripping buffer (Restore) was from Pierce. Human embryonic kidney (HEK) 293 cells (ATCC collection) were cultured on 10 cm dishes in Dulbecco's modified Eagle's medium containing 10% fetal bovine serum (Gibco). Transient transfection of HEK 293 cells was performed using a calcium chloride protocol. Materials for mammalian tissue culture were from Greiner. Insect cell expression system and related material were from Invitrogen and were used as recommended by the manufacturer. Molecular biology techniques were performed using standard protocols. Site-directed mutagenesis was performed using a QuikChange kit (Stratagene) following the instructions provided by the manufacturer. DNA constructs used for transient transfection were purified from bacteria using a Qiagen plasmid Mega kit according to the manufacturer's protocol. DNA sequences were verified by automatic DNA sequencing (Applied Biosystems 3100 Genetic Analyzer). Commercial small molecular weight compounds used in preliminary screenings were obtained from Chembridge (San Diego, USA), Maybridge (Tintagel, UK) or Specs (Rijswijk, Netherlands).

### Expression and Purification of Protein Kinases.

For the expression and purification of protein kinases fused to GST, pEBG2T derived plasmids were transfected by a modified calcium phosphate method (10 μg plasmid/10 cm dish) into HEK293 cells, the cell media exchanged after 20 h and the cells lysed after 20 h in a buffer containing 50 mM Tris-HCl pH 7.5, 1 mM EGTA, 1 mM EDTA, 1% (w/v) Triton X-100, 1 mM sodium orthovanadate, 50 μM sodium fluoride, 5 mM sodium pyrophosphate, 0.27 M sucrose, 0.1% β-mercaptoethanol, and 1 tablet of protease inhibitor cocktail per 50 ml of buffer. Lysates were frozen in liquid nitrogen and kept at −80° C. until required. Purification involved incubation of the cleared lysate with glutathione sepharose, 4 washes with 0.5 M NaCl in lysis buffer, followed by 10 washes with a buffer containing 50 mM Tris-HCl, 0.5 mM EGTA and 0.1% β-mercaptoethanol, and elution with the same buffer containing 20 mM glutathione. GST fusion proteins were aliquoted, snap frozen in liquid nitrogen and kept at −80° C. until use. Purity at this stage was above 85% as estimated by SDS-PAGE and staining with Coomassie brillant blue R250. PDK1 was expressed from pEBG-2T-PDK1, S6K1 from pEBG-2T-S6K1-T2[Thr412Glu] and pEBG-2T-S6K1-T2[Thr412Ala], SGK1 from pEBG-2T-SGK1-ΔN[Ser422Asp], PKBα from pEBG-2T-PKBα[Ser473Asp] (Biondi et al., 2001), PKCζ from pEBG-2T-PKCζ, PRK2 from pEBG-2T-PRK2-ΔN (Balendran et al., 2000).

For the spectrofluorometer assay, the PDK1 protein used was the catalytic domain of PDK1 (50-359) produced in SF9 insect cells using baculovirus expression technology (Invitrogen). PDK1 was cloned in pFastbac-HT plasmid (NcoI-KpnI sites) taking advantage of an endogenous NcoI site in the PDK1 DNA sequence. The codon corresponding to aminoacid 360 was replaced by a stop codon. The protein was produced and purified essentially as described previously (Biondi et al., 2002) with differences due to the use of TEV protease to cleave the His-tag. TEV protease (Ser205Val) was expressed from pET19b plasmid kindly supplied by A. Scheidig (University of Saarland). After cleavage, the purified PDK1 protein contained an extra Gly residue preceding residue 50 of PDK1 and was homogeneous as verified by SDS-PAGE, IEF and the ability to form crystal needles. PDK1 [Phe224Trp] and PDK1 [Thr226Trp] were produced in the same pFastbac-HT PDK1 50-360 vector, expressed and purified as described for the catalytic domain of PDK1. The PDK1-PIF chimera was also produced in the pFastbac-HT 50-360 fused in frame to the last 48 aminoacids from PRK2.

### Protein Kinase Activity Tests.

Protein kinase activity tests were performed essentially as previously described (Balendran et al., 2000; Biondi et al., 2000; Biondi et al., 2001). Substrates used were either MBP (for PKCζ or polypeptides (Crosstide for PKB, S6K, SGK and PRK2; T308tide for PDK1, and Kemptide for PKA). PKA (Sigma) was measured in the presence of 100 μM cAMP. Alternatively, assays were performed in a 96 well format, aliquots spotted on p81 phosphocellulose papers (Whatmann), washed in 0.01% phosphoric acid, dried, and then exposed and analysed using Phospholmager technology (Storm, Molecular Dynamics). Activity measurements were performed in duplicates with less than 10% difference between duplicate pairs. Experiments were repeated at least twice, although most of the experiments were repeated multiple times, with similar results.

PDK1 activity assay was performed in a 20 μl mix containing 50 mM Tris-HCl pH 7.5, 0.05 mg/ml BSA, 0.1% β-mercaptoethanol, 10 mM MgCl₂, 100 μM [γ³²P]ATP (5-50 cpm/pmol), 0.003% Brij, 150 ng PDK1, and T308tide (from 0.05 to 1 mM). PDK1 specific activity was approximately 5 U/mg (when measured at 1 mM T308tide). The effect of small compounds on PDK1 was repeated with proteins from different purification batches and with different protein constructs, comprising the full length protein or the catalytic domain alone, with similar results. Nevertheless, some differences were observed in the maximal activation of PDK1 between different batches of the same PDK1 construct (4× to 7× maximal activation). This variability may be due to the PIFtide/compound-stimulated autophosphorylation of the activation loop in certain preparations having less than a 1:1 molar phosphorylation stoichiometry at this site.

Phosphorylation of 56K1-T2[T412E] and SGK1-ΔN[S422D] by PDK1 in vitro was performed as previously described (Biondi et al., 2001), with the sole difference that the reaction mix included 1% DMSO, and, where indicated, compound 1. In order to measure the effect of compound 1 on the activity and the level of activation loop phosphorylation of S6K1 in cells, pEBG2T vectors coding for GST-S6K1-T2[Thr412Glu] and GST-S6K1-T2[Thr412Ala] were transfected into HEK 293 cells (3.5 cm dishes), and the cells serum starved during 16 h previous to treatment with compound 1 (200 μM). Cells were lysed 36 h after transfection, cleared by centrifugation and incubated with glutathione sepharose. The resin was washed two times with lysis buffer supplemented with 0.250 M NaCl, followed by 2 washes with a buffer containing 50 mM Tris-HCl, 0.1 mM EGTA and 0.1% β-mercaptoethanol. The GST-fusion proteins were eluted by the addition of glutathione 20 mM and the mix was cleared from resin by filtration through Spin-X tubes. The resulting proteins were used both for western-blotting and for activity measurements. Similar decrease in S6K activity was observed when the cells were treated with IGF1 for 20 min prior to cell lysis. Activity assays of the immunoprecipitated S6K, in the presence of resin, were done under agitation.

### In Silico Screening of Small Molecular Weight Compound Databases.

Searches of the Maybridge database of small compounds were performed using the flexible search routine of Unity 4.3 (Tripos), which adjusts the conformations in the course of the search and treat, to some extent, compound flexibility. The structural base for the conducted searches was the PKA structure with the two interacting partners being the hydrophobic HM/PIF-pocket and its natural C-terminal ligand (Cys343 to Phe 350). The aminoacids Gln35, Leu74, Lys76, Val79, Val80, Ile85, Glu86, Leu89, Leu92, Arg93, Lys111, Leu116 were considered to be relevant for limiting the two hydrophobic regions in the PIF-pocket. Different ligand searches were performed, which included Phe347 and Phe350, and possible hydrogen bond donor oxygens. Restrictions were applied with either an exclusion volume related to Leu116 or to distance constrains between the benzene rings. Depending on the restrictions applied, a few hundred to several thousands compounds were retrieved from each search, without a scoring system. A diverse set of compounds were then tested in vitro and the results used for further selection of compounds in an iterative manner.

### Synthesis and Characterization of Compounds.

Compound 1 (3-(p-Chlorophenyl)-3-oxo-1-phenyl)-propyl sulfanyl acetic acid): freshly distilled benzaldehyde was dissolved in 95% ethanol and sodium hydroxide was added. 4′-chlorophenacetone was added and the mixture stirred for 1.5 h at 22° C. The solid chalcone product was then filtered and washed with 70% ice-cold ethanol in water followed by water. The product was dissolved in 3 ml acetonitrile and 1.4 mmol of triethylamine and 1.2 mmol of fresh thioglycolic acid were added to the mixture at RT. After stirring at RT for 2 h, the solvent was removed by rotary evaporation and the crude product re-dissolved in ethylacetate. The organic layer was washed three times with 1 N HCl, dried over MgSO₄ and the ethylacetate removed by rotary evaporation. The crude product, appearing as a slightly yellow oil was purified over a silica gel flash column using n-hexane/ethylacetate/formic acid (73:24:3 v/v/v) as a mobile phase. Fractions containing the desired compound were pooled and the mobile phase removed by rotary evaporation, after which compound 1 was obtained as a white solid (yield: 70%): mp: 123-124° C.; TLC (n-hexane:ethylacetate:formic acid, 73:24:3 v/v/v): R_{f}=0.32;

For compound 2 (3-(p-Chlorophenyl)-3-oxo-1-phenyl)-propyl sulfanyl acetic acid methyl ester), the same chalcone precursor as for compound 1 was reacted in the following way: 1 mmol of the chalcone was dissolved in 3 ml methanol and thioglycolic acid (1.5 mmol) was added. The mixture was stirred under reflux for 24 h, then a few drops of acetic acid were added, and stirring under reflux was continued for another 16 h. The methanol was removed by rotary evaporation and the crude product purified by Flash chromatography using a mobile phase of n-hexane/ethylacetate (3:1 v/v). Compound 2 was obtained as a yellow oil (yield: 64%): TLC (n-hexane:ethylacetate:formic acid, 73:24:3 v/v/v): R_{f}=0.48.

The identity of the products were confirmed by ¹H-NMR, IR analysis and mass spectrometry.

Compounds according to general formula I are achievable by the above described methods starting from the educts with desired substitution pattern or by the application of other methods described known to the expert skilled in the art.

### REFERENCES FOR EXAMPLE 1

Bayliss, R., Sardon, T., Vernos, I. and Conti, E. (2003) Structural basis of Aurora-A activation by TPX2 at the mitotic spindle. Mol Cell, 12, 851-862.
Balendran, A., Biondi, R. M., Cheung, P. C., Casamayor, A., Deak, M. and Alessi, D. R. (2000) A 3-phosphoinositide-dependent protein kinase-1 (PDK1) docking site is required for the phosphorylation of protein kinase Czeta (PKCzeta) and PKC-related kinase 2 by PDK1. J Biol Chem, 275, 20806-20813.
Biondi, R. M., Cheung, P. C., Casamayor, A., Deak, M., Currie, R. A. and Alessi, D. R. (2000) Identification of a pocket in the PDK1 kinase domain that interacts with PIF and the C-terminal residues of PKA. Embo J, 19, 979-988.
Biondi, R. M., Kieloch, A., Currie, R. A., Deak, M. and Alessi, D. R. (2001) The PIF-binding pocket in PDK1 is essential for activation of S6K and SGK, but not PKB. Embo J, 20, 4380-4390.
Biondi, R. M., Komander, D., Thomas, C. C., Lizcano, J. M., Deak, M., Alessi, D. R. and van Aalten, D. M. (2002) High resolution crystal structure of the human PDK1 catalytic domain defines the regulatory phosphopeptide docking site. Embo J, 21, 4219-4228.
Biondi, R. M. (2004) Phosphoinositide-dependent protein kinase 1, a sensor of protein conformation. Trends Biochem Sci, 29, 136-142.
Biondi, R. M., Cheung, P. C., Casamayor, A., Deak, M., Currie, R. A. and Alessi, D. R. (2000) Identification of a pocket in the PDK1 kinase domain that interacts with PIF and the C-terminal residues of PKA. Embo J, 19, 979-988.
Biondi, R. M., Kieloch, A., Currie, R. A., Deak, M. and Alessi, D. R. (2001) The PIF-binding pocket in PDK1 is essential for activation of S6K and SGK, but not PKB. Embo J, 20, 4380-4390.
Biondi, R. M., Komander, D., Thomas, C. C., Lizcano, J. M., Deak, M., Alessi, D. R. and van Aalten, D. M. (2002) High resolution crystal structure of the human PDK1 catalytic domain defines the regulatory phosphopeptide docking site. Embo J, 21, 4219-4228.
Biondi, R. M. and Nebreda, A. R. (2003) Signalling specificity of Ser/Thr protein kinases through docking-site-mediated interactions. Biochem J, 372, 1-13.
Blume-Jensen, P. and Hunter, T. (2001) Oncogenic kinase signalling. Nature, 411, 355-365.
Cohen, P. (2002) Protein kinases—the major drug targets of the twenty-first century? Nat Rev Drug Discov, 1, 309-315.
Collins, B. J., Deak, M., Arthur, J. S., Armit, L. J. and Alessi, D. R. (2003) In vivo role of the PIF-binding docking site of PDK1 defined by knock-in mutation. Embo J, 22, 4202-4211.
Collins, B. J., Deak, M., Murray-Tait, V., Storey, K. G. and Alessi, D. R. (2005) In vivo role of the phosphate groove of PDK1 defined by knockin mutation. J Cell Sci, 118, 5023-5034.
Dajani, R., Fraser, E., Roe, S. M., Young, N., Good, V., Dale, T. C. and Pearl, L. H. (2001) Crystal structure of glycogen synthase kinase 3 beta: structural basis for phosphate-primed substrate specificity and autoinhibition. Cell, 105, 721-732.
Etchebehere, L. C., Van Bemmelen, M. X., Anjard, C., Traincard, F., Assemat, K., Reymond, C. and Veron, M. (1997) The catalytic subunit of Dictyostelium cAMP-dependent protein kinase—role of the N-terminal domain and of the C-terminal residues in catalytic activity and stability. Eur J Biochem, 248, 820-826.
Frame, S., Cohen, P. and Biondi, R. M. (2001) A common phosphate binding site explains the unique substrate specificity of GSK3 and its inactivation by phosphorylation. Mol Cell, 7, 1321-1327.
Frodin, M., Antal, T. L., Dummler, B. A., Jensen, C. J., Deak, M., Gammeltoft, S, and Biondi, R. M. (2002) A phosphoserine/threonine-binding pocket in AGC kinases and PDK1 mediates activation by hydrophobic motif phosphorylation. Embo J, 21, 5396-5407.
Frodin, M., Jensen, C. J., Merienne, K. and Gammeltoft, S. (2000) A phosphoserine-regulated docking site in the protein kinase RSK2 that recruits and activates PDK1. Embo J, 19, 2924-2934.
Gonfloni, S., Williams, J. C., Hattula, K., Weijland, A., Wierenga, R. K. and Superti-Furga, G. (1997) The role of the linker between the SH2 domain and catalytic domain in the regulation and function of Src. Embo J, 16, 7261-7271.
Huang, X., Begley, M., Morgenstern, K. A., Gu, Y., Rose, P., Zhao, H. and Zhu, X. (2003) Crystal structure of an inactive akt2 kinase domain. Structure (Camb), 11, 21-30.
Huse, M. and Kuriyan, J. (2002) The conformational plasticity of protein kinases. Cell, 109, 275-282.
Johnson, L. N. and Lewis, R. J. (2001) Structural basis for control by phosphorylation. Chem Rev, 101, 2209-2242.
Knighton, D. R., Xuong, N. H., Taylor, S. S. and Sowadski, J. M. (1991) Crystallization studies of cAMP-dependent protein kinase. Cocrystals of the catalytic subunit with a 20 amino acid residue peptide inhibitor and MgATP diffract to 3.0 A resolution. J Mol Biol, 220, 217-220.
Komander, D., Kular, G., Deak, M., Alessi, D. R. and van Aalten, D. M. (2005) Role of T-loop phosphorylation in PDK1 activation, stability, and substrate binding. J Biol Chem, 280, 18797-18802.
Newton, A. C. (2003a) Regulation of the ABC kinases by phosphorylation: protein kinase C as a paradigm. Biochem J, 370, 361-371.
Newton, A. C. (2003b) Regulation of the ABC kinases by phosphorylation: protein kinase C as a paradigm. Biochem J, 370, 361-371.
Parker, P. J. and Parkinson, S. J. (2001) AGC protein kinase phosphorylation and protein kinase C. Biochem Soc Trans, 29, 860-863.
Pawson, T. and Scott, J. D. (2005) Protein phosphorylation in signaling—50 years and counting. Trends Biochem Sci, 30, 286-290.
Pearl, L. H. and Barford, D. (2002) Regulation of protein kinases in insulin, growth factor and Wnt signalling. Curr Opin Struct Biol, 12, 761-767.
Sicheri, F., Moarefi, I. and Kuriyan, J. (1997) Crystal structure of the Src family tyrosine kinase Hck. Nature, 385, 602-609.
Smith, K. J., Carter, P. S., Bridges, A., Horrocks, P., Lewis, C., Pettman, G., Clarke, A., Brown, M., Hughes, J., Wilkinson, M., Bax, B. and Reith, A. (2004) The structure of MSK1 reveals a novel autoinhibitory conformation for a dual kinase protein. Structure (Camb), 12, 1067-1077.
Xu, W., Harrison, S. C. and Eck, M. J. (1997) Three-dimensional structure of the tyrosine kinase c-Src. Nature, 385, 595-602.
Yang, J., Cron, P., Thompson, V., Good, V. M., Hess, D., Hemmings, B. A. and Barford, D. (2002) Molecular mechanism for the regulation of protein kinase B/Akt by hydrophobic motif phosphorylation. Mol Cell, 9, 1227-1240.

### Example 2

### A Method of Activation of Agc Kinases by Linker and Hydrophobic Motif Phosphorylation Sites

### Introduction to Example 2

### Introduction

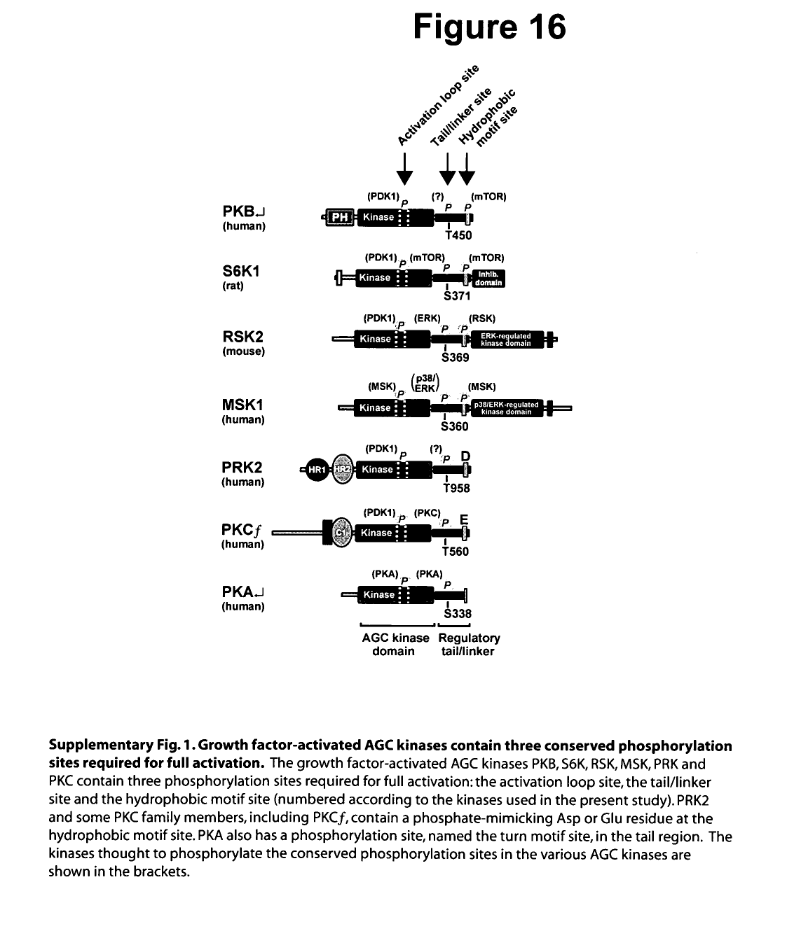

A significant portion of growth factor/insulin signalling is mediated by a functionally diverse, but structurally related group of protein kinases that belong to the AGC kinase family. The group, here called the growth factor-activated AGC kinases, includes protein kinase B (PKBα-γ or AKT1-3), p70 ribosomal S6 kinase (S6K1,2), p90 ribosomal S6 kinase (RSK1-4), mitogen- and stress-activated protein kinase (MSK1,2) and several members of the protein kinase C (PKC) family. These kinases regulate cellular division, growth, survival, metabolism, motility and differentiation and several are implicated in human disease. The kinases function in partly distinct signalling pathways, such as the phosphoinositide 3-kinase (PI3-K) pathway (PKB and S6K) (Kozma and Thomas, 2002), MAP kinase pathways (RSK and MSK) (Hauge and Frodin, 2006), in calcium/lipid signalling (PKC) (Parekh et al., 2000; Newton, 2003), or in Rho GTPase signalling (PRK2) (Parekh et al., 2000). The responsiveness to distinct upstream pathways is partly due to distinct signalling modules flanking the kinase domain in the various kinases (FIG. 16).

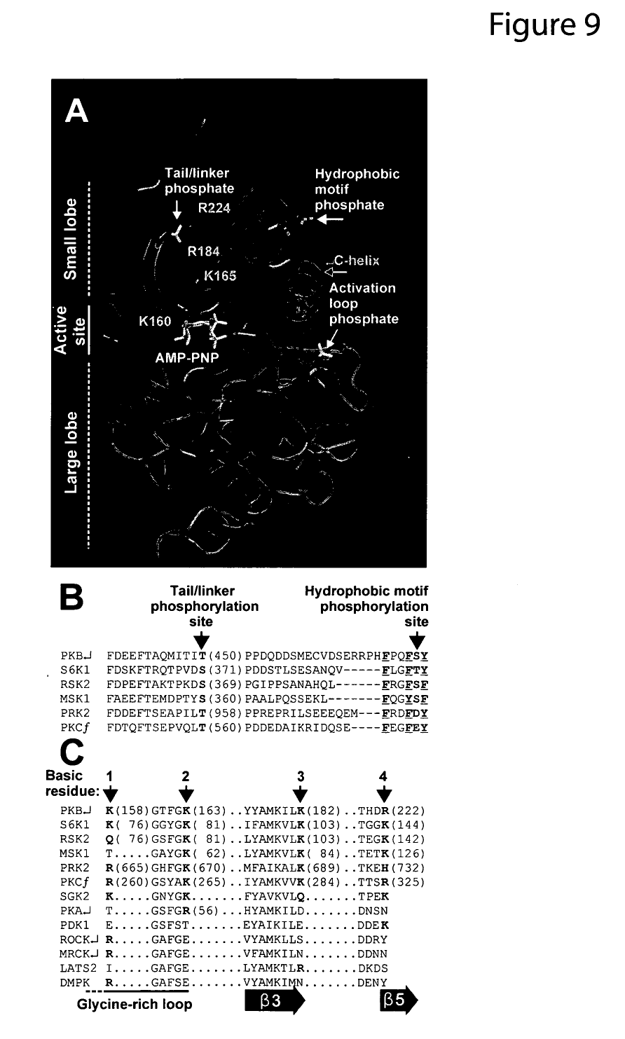

In addition to the divergent regulation, the growth factor-activated AGC kinases share a common core mechanism of activation, which is based on 3 conserved phosphorylation sites. Viewed simplistically, the flanking signalling modules serve to induce proper phosphorylation of these phosphorylation sites. The 3 sites are located in the activation loop in the kinase domain, in the middle of a tail/linker region C-terminally to the kinase domain, and within a hydrophobic motif (HM) at the end of the tail region, respectively (FIG. 9B and FIG. 16). The various kinases thought to target the 3 phosphorylation sites are indicated in FIG. 16.

Intense efforts have recently established the mechanism of action of the HM and the activation loop phosphorylation sites (Biondi et al., 2000; Frodin et al., 2002; Yang et al., 2002a; Yang et al., 2002b; Smith et al., 2004). Phosphorylation of the HM triggers interaction of the phosphate with a phosphate-binding site in the small lobe of the kinase domain, providing the final binding energy required for the aromatic residues of the HM to interact with and stabilize a nearby hydrophobic pocket, which is disordered in the inactive kinase. Stabilization of the so-called αC-helix in this pocket is thought to be of key importance, since this helix contains key residues for regulation of phosphotransferase activity and since it may aid to stabilize the activation loop in an optimal conformation. In RSK and S6K, the phosphorylated HM additionally functions as a phosphorylation-dependent docking site that recruits and activates the activation loop kinase PDK1 (Frodin et al., 2000; Biondi et al., 2001). The phosphate in the activation loop stimulates kinase activity by binding to basic residues in loops within the active site, which helps position catalytic residues (Knighton et al., 1991). Individual phosphorylation of the HM and the activation loop induces negligible and low level activation, respectively. However, in combination, the two phosphorylation events synergistically stimulate kinase activity (Alessi et al., 1996; Frodin et al., 2002; Yang et al., 2002b). One mechanism for this cooperativity is thought to derive from the ability of both phosphates to promote interaction between the αC-helix and the activation loop, leading to mutual stabilization of these key regulatory structures and promotion of the active, closed conformation of the kinase domain.

The phosphorylation site in the middle of the tail is the most poorly characterized of the 3 conserved sites, yet its mutation significantly reduces kinase activity and in some AGC kinases also HM phosphorylation (Moser et al., 1997; Bellacosa et al., 1998; Weng et al., 1998; Parekh et al., 2000; Newton, 2003; Matsuzaki et al., 2004; McCoy et al., 2004). In RSK and MSK, the site is phosphorylated by ERK and p38 or ERK, respectively, during activation (Dalby et al., 1998; McCoy et al., 2004). In S6K, the site may be phosphorylated by mTOR and displays high basal phosphorylation, which is increased 2-fold in response to growth factor/insulin (Saitoh et al., 2002). In PKB, the site is constitutively phosphorylated by an unknown kinase (Alessi et al., 1996). In PKCs, the site is thought to be autophosphorylated during maturation to the latent catalytically competent conformation (Parekh et al., 2000; Newton, 2003).

The mechanism of action of the phosphorylation site in the middle of the tail is elusive. The AGC protein kinase A (PKA) also contains a phosphorylation site in the middle of its tail region, known as the “turn motif” site, since the phosphate binds nearby residues within the tail and thereby stabilizes a turn in the tail. It has been widely assumed that the tail phosphate in the growth factor-activated AGC kinases performs the same function. Consequently, the site is also known as the turn motif in these kinases and has been aligned with the turn motif of PKA (Yang et al., 2002b; see review by Newton, 2003; or Roux and Blenis, 2004).

Here, we report the mechanism(s) whereby the tail phosphorylation site activates PKBα, S6K1, RSK2, MSK1, PRK2 and PKCζ, which represent 6 of the 7 families of growth factor-activated AGC kinases. We report that this phosphorylation site is not equivalent to the turn motif site of PKA, but rather corresponds to Glu333 of PKA. In the growth factor-activated AGC kinases, the tail phosphate binds a phosphoSer/Thr-binding site in the kinase domain near the hydrophobic pocket, serving to deliver the HM to its binding site in a zipper-like manner. Our results suggest that the tail phosphate thereby synergistically enhances kinase activation via HM-mediated stabilization of the αC-helix and, in a subset of the kinases, also controls the phosphorylation state of the HM. Based on these findings, it could be considered referring to the tail site in growth factor-activated AGC kinases as the Z (zipper) site instead of the turn motif site.

Our results suggest that the overall mechanism described is a key feature in activation of up to 26 human AGC kinases that has been widely conserved during evolution.

### Results

### A Potential Binding Site for the Tail PhosphoSer/Thr is Widely Conserved in the Catalytic Domain of AGC Kinases

PKBβ has been crystallized in an active conformation showing the HM bound to the hydrophobic pocket (Yang et al., 2002a). However a large portion of the tail region, including the tail phosphorylation site (T451), was not visible in the structure. The visible tail region ends with a short helix near the small lobe of the kinase domain.

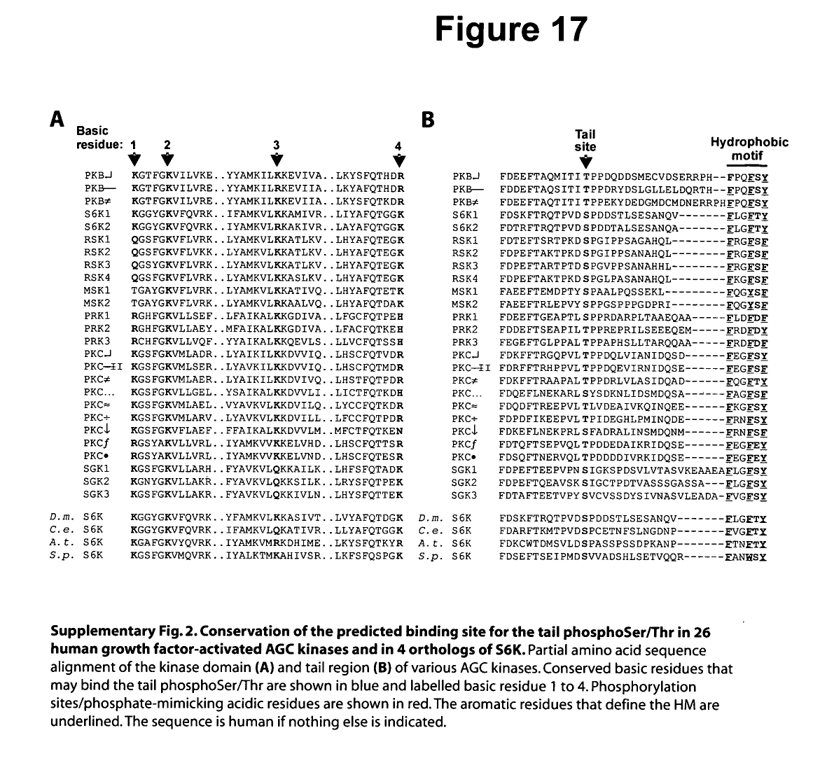

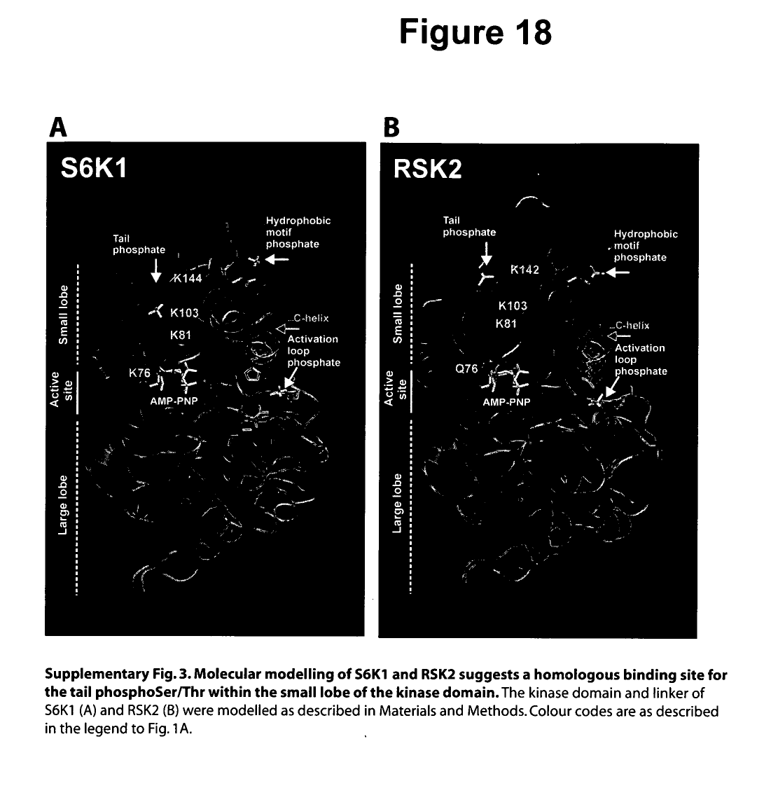

Our initial modelling analysis did not support the possibility of a binding site for phosphoT451 within the tail region. We therefore analysed the small lobe of PKB for binding sites for phosphoT451 using the programs GRASP and GRID. Among various potential sites, we focused on the most interesting site, located above the ATP-binding, glycine-rich loop and formed by 4 basic residues, K160, K165, R184 and R224 (hereafter referred to as basic residues 1 to 4) of which the first two are part of the glycine-rich loop. Ab initio modelling of the non-crystallized region of the tail suggested that the phosphate of T451 might be located in the middle of this basic cluster. The location appeared energetically favourable, since the phosphate remained in the site during dynamics simulations on the model, constantly interacting with 2 or 3 of the basic residues which differed over time (FIG. 9A). The 4 basic residues are conserved in all 23 members of the PKB, S6K, RSK, MSK, PRK and PKC families (FIG. 9C and FIG. 17). They are also conserved in the 3 members of the SGK family of growth factor-activated AGC kinases, which have a tail phosphorylation site (Kobayashi et al., 1999), required for full kinase activity (C. J. Jensen and M. Frodin, unpublished observation). Finally, the tail site and the basic residues are co-conserved during evolution as illustrated by S6K orthologues from D. melanogaster, C. elegans, A. thaliana, and S. pombe (FIG. 17). Modelling of S6K1 and RSK2 supported the existence of a phosphate-binding site homologous to that of PKBβ (FIG. 18). The basic residues are poorly conserved in AGC kinases not thought to contain a tail phosphorylation site (PDK1, ROCK, MRCK, LATS and DMPK, FIG. 9C).

Thus, modelling and sequence conservation suggested that in the growth factor-activated AGC kinases, the tail phosphoSer/Thr interacts with a phosphate-binding site within the kinase domain, implying a different role of this phosphorylation site from that of the turn motif site in PKA. The functional characterization presented below suggests that the tail phosphate promotes zipper-like binding of the tail and HM to the kinase domain, aimed at controlling activation of the kinases by the HM.

### Role of the Tail Site in Phosphorylation and Activation of AGC Kinases In Vivo

We first characterized further the importance of the tail site in the growth factor-activated AGC kinases. PKBα, S6K1, RSK2 and MSK1 were purified from transiently transfected COS7 cells exposed to an appropriate stimulus. PRK2, including the truncated mutant used here, PRK2_{Δ1-500}, was active in non-stimulated COS7 cells and was therefore purified from non-treated cells.

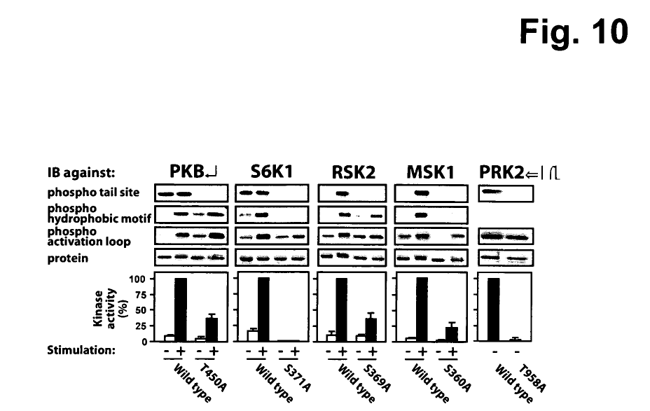

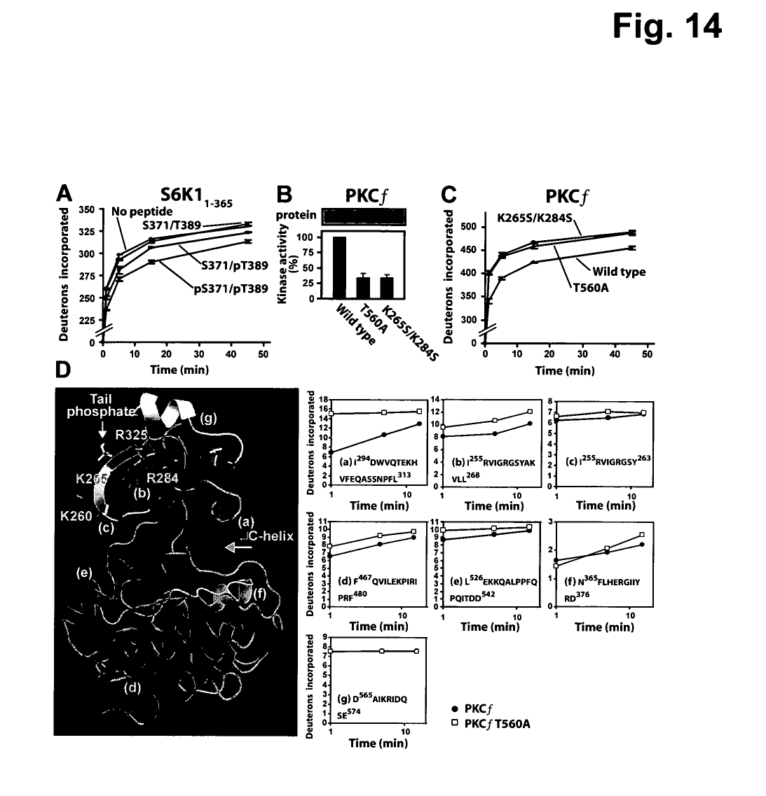

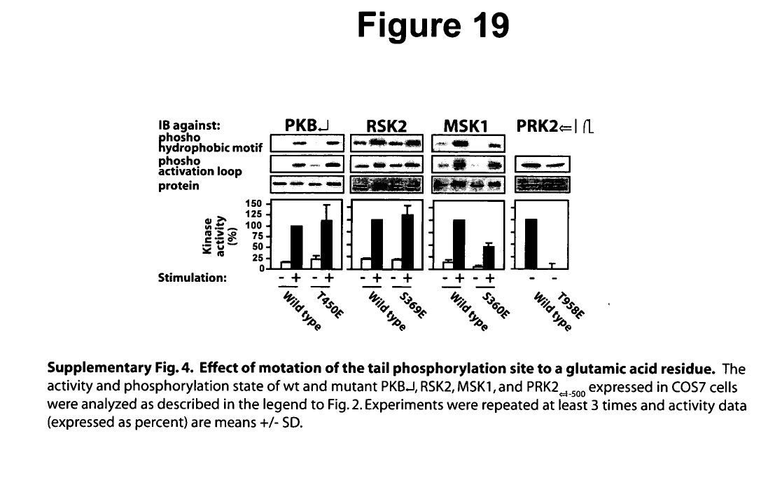

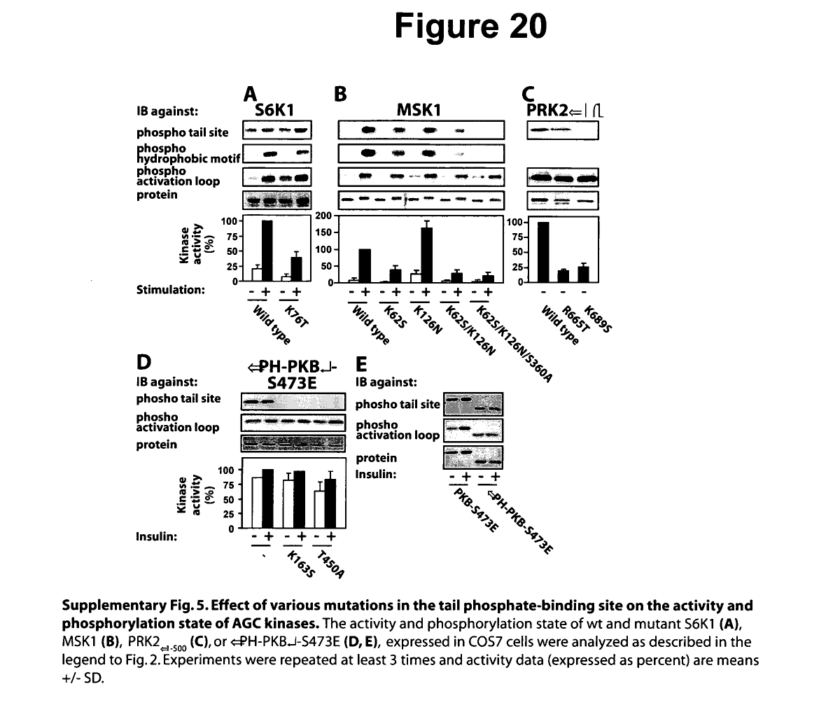

Immunoblotting with phosphospecific antibodies showed that the tail site was constitutively phosphorylated in PKBα, PRK2 and S6K1 (sometimes a 2-fold induction was observed in S6K1, FIG. 20A) and strongly induced in RSK2 and MSK1 following stimulation (FIG. 10). Mutation of the tail site to Ala reduced activation from ≈60% in PKBα to ≈98% in S6K1 (FIG. 10). Furthermore, in S6K1, MSK1 and RSK2, mutation of the tail site reduced phosphorylation of the HM with moderate to profound effects. In PKBα, mutation of the tail site modestly enhanced phosphorylation of the HM and the activation loop. Mutation of the tail site to phosphate-mimicking Glu could substitute for phosphorylation in PKBα and RSK2, partially in MSK1, but not in PRK2 (FIG. 19), similar to findings with S6K1 (Moser et al., 1997). These results further establish the importance of the tail site in PKB, S6K, RSK and MSK and report its role in the PRK family (and in PKCζ, FIG. 14) for the first time.

### The Predicted Binding Site for the Tail Phosphate is Essential for Normal Activation and Phosphorylation of AGC Kinases In Vivo

We next mutated the predicted binding site for the tail phosphate by introducing amino acids that are present at the corresponding positions in AGC kinases without a tail site, assuming that such mutations would not compromise tertiary structure. The assumption was supported by normal expression of nearly all mutants. Analysis of 76 point-mutants suggested that the predicted binding site is functional in all growth factor-activated AGC kinases and identified the most important individual or combinations of basic residues in the various AGC kinase subfamilies.

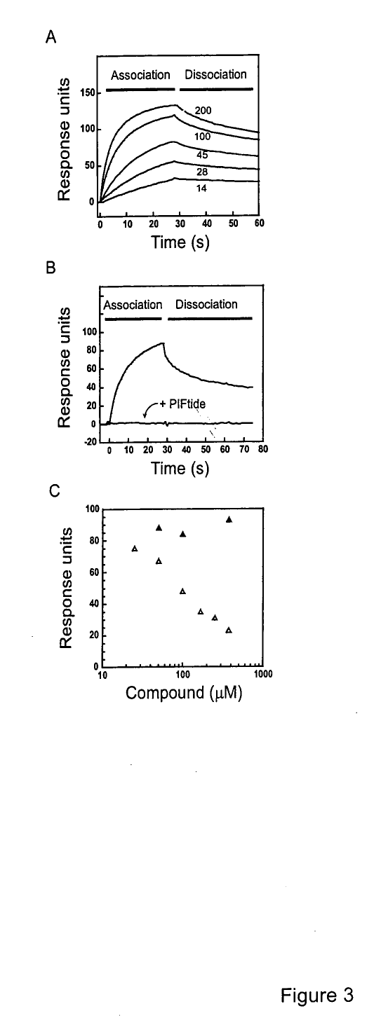

In PKBα, quadruple mutation of the four basic residues (as in PKBα-K158T/K163S/K182S/R222N), reduced kinase activity by ≈40% (FIG. 3A), comparable to the ≈60% reduction resulting from mutation of the tail site T450 (FIG. 10). Moreover, phosphorylation of T450 was considerably reduced in PKBα-K158T/K163S/K182S/R222N, suggesting that the binding of phosphoT450 to the basic residues protects it from dephosphorylation. The finding that PKBα-K158T/K163S/K182S/R222N had somewhat higher activity than PKBα-T450A likely results from residual phosphorylation at T450 in the former mutant. The residual phosphate may interact with the introduced Thr, Ser and Asn residues and induce a small degree of activation, as these amino acids can bind phosphoSer/Thr, although with lower affinity than Lys and Arg.

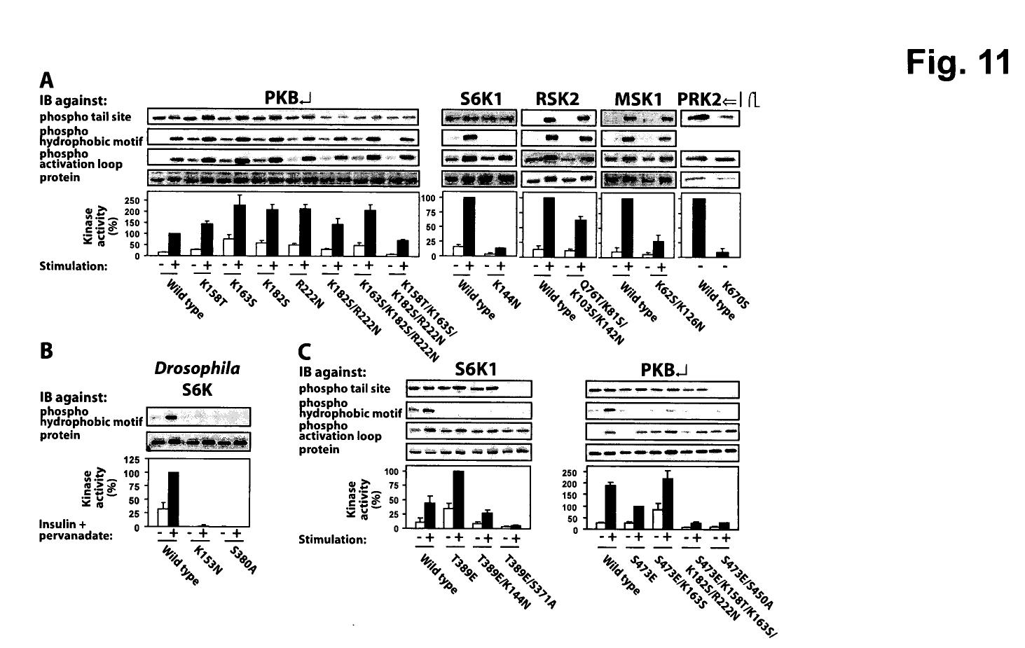

Surprisingly, single to triple mutation of the basic residues in PKBα resulted in significantly increased basal and insulin-stimulated kinase activity, which apparently resulted from increased phosphorylation of the HM and the activation loop (FIG. 11A). These mutations also increased the biological activity of PKB, since NIH 3T3 cells virally transduced with PKBα-K163S or PKBα-K182S showed a small, but significant increase in colony formation in sparsely seeded cultures, as compared to cells transduced with wt PKBα (C. Hauge and M. Frodin, unpublished observation).

In S6K1, mutation of basic residue 4 reduced kinase activity by ≈85% (FIG. 11A), comparable to the ≈98% reduction caused by mutation of the tail site S371 (FIG. 10). Furthermore, mutation of basic residue 4 profoundly reduced phosphorylation in the HM and moderately in the activation loop, similar to the effects obtained by mutation of the tail site. Mutation of basic residue 1 reduced kinase activity and HM phosphorylation by ≈60% (FIG. 20A). In RSK2, individual mutation of basic residue 1 to 4 had negligible effect, but mutation of all four residues reduced kinase activity by ≈40%, comparable to the ≈60% reduction caused by mutation of the tail site S369, and slightly reduced HM phosphorylation. In MSK1, double mutation of basic residues 2 and 4 reduced kinase activity to almost the same low level as that obtained after mutation of the tail site S360. The double mutation also significantly reduced phosphorylation of the HM, as did mutation of the tail site. In PRK2, individual mutation of basic residue 2 (or 1 or 3, FIG. 20C), reduced kinase activity to similarly low levels as that obtained by mutation of the tail site T958. Mutation of the basic residues caused a profound reduction in phosphorylation of T958, suggesting that the binding of phosphoT958 to these residues protects it from dephosphorylation. In the various kinases, the effects of mutating the tail site and the critical basic residues were not additive (FIG. 20B and data not shown), indicating that the tail phosphate and its predicted binding site regulate kinase activity by the same mechanism.

The role of the tail phosphate appears highly conserved during evolution, since mutation of the tail site S380 and basic residue 4 (K153) abolished kinase activity and HM phosphorylation of Drosophila S6K expressed in Drosophila S2 cells (FIG. 11B).

Since mutation of the tail phosphate-binding site decreased the phosphorylation of HM in S6K1 and MSK1, it could not be determined whether the site affected kinase activity by a mechanism other than regulation of HM dephosphorylation. To render the HM insensitive to phosphatases, we generated mutants with Glu in the HM phosphorylation site (S6K1-T389E, MSK1-S376E). S6K1-T389E possessed higher activity than wt S6K1 (FIG. 11C), in accordance with previous findings (Weng et al., 1998). More importantly, mutation of basic residue 1 or 4 in S6K1-T389E reduced kinase activity to the same extent as did these mutations in wt S6K1 without affecting the phosphorylation state (FIG. 11C and data not shown). Similar results were obtained with MSK1-S376E (data not shown). Thus, the tail phosphate-binding site can activate S6K1 and MSK1 by a mechanism distinct from the one protecting the HM from dephosphorylation.

In PKBα with Glu at the HM phosphorylation site (PKBα-S473E), the quadruple mutation (K158T/K163S/K182S/R222N) and the K163S mutation of the tail phosphate-binding site had the same effects as in wt PKBα (FIG. 11C). This agrees with the results obtained with wt PKBα that severe disruption of the interaction between the tail phosphate and its binding site precludes full kinase activity and that high levels of phosphorylation in the activation loop and Glu in the HM cannot compensate for the disrupted interaction. In PKBα-S473E with deletion of the PH domain (ΔPH-PKBα-S473E), the K163S mutation did not increase phosphorylation or kinase activity of PKBα (FIG. 20D), further suggesting that this mutation stimulates PKBα activity by inducing hyperphosphorylation, which may occur at the plasma membrane. In ΔPH-PKBα-S473E, mutation of the tail site had less effect on kinase activity compared to full length PKBα (FIG. 20D), possibly because ΔPH-PKBα-S473E was less phosphorylated at the tail site (FIG. 20E), which suggests that membrane localization of PKBα promotes phosphorylation of the tail site. However, these experiments should be interpreted with caution, since deletion of the PH domain likely alters the conformation of PKB.

We conclude that the tail phosphate interacts with a binding site widely conserved among growth factor-activated AGC kinases. We propose that the tail phosphate thereby functions as a molecular zipper that helps deliver the HM to its binding site and stabilize it there, which has two consequences. Firstly, in all of the kinases this directly stimulates kinase activity, presumably by stabilization of the active kinase conformation. Secondly, in a subset of the kinases this controls the phosphorylation state of the HM, presumably by restricting its exposure to phosphatases and kinases. While a functional tail phosphate-binding site is conserved in all of the kinases studied, the key basic residues involved in forming the site varies somewhat among the different AGC kinase families.

### Further Evidence of the Tail Phosphate-Binding Site

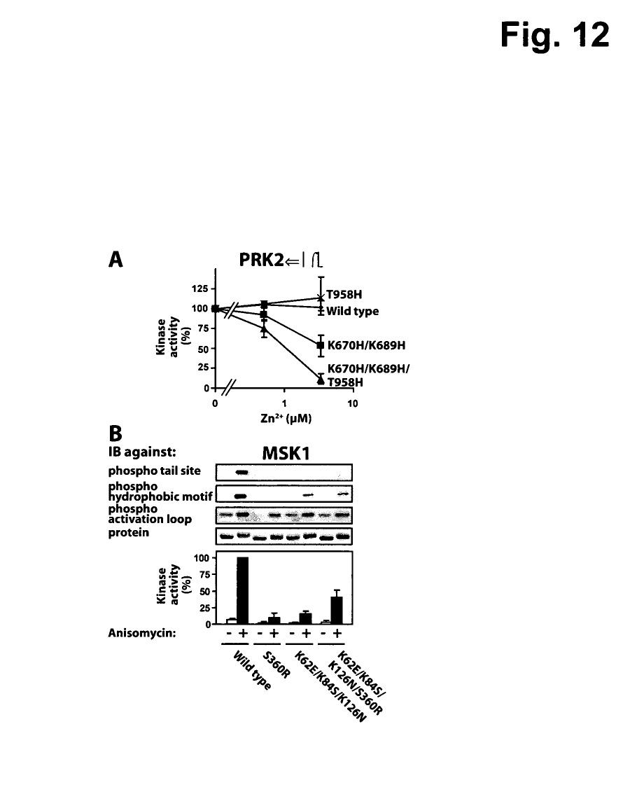

If the tail phosphate and the basic residues are indeed within interaction distance, it might be possible to engineer a Zn²⁺-binding site by replacing the tail site Ser/Thr and the basic residues with histidines. A Zn²⁺-binding site may be detected by Zn²⁺-dependent modulation of enzymatic function, most often inhibition due to distortion of protein structure. Zn²⁺ up to 3.3 μM had no effect on the activity of wt PRK2 or PRK2 with His in the tail site (PRK2-T958H) (FIG. 12A). By contrast, PRK2 with His in place of basic residues 2 and 3 (PRK2-K670H/K689H) was inhibited by Zn²⁺ at 3.3 μM, but not at lower concentrations. Thus, the close proximity of basic residues 2 to 4 (wt PRK2 has His at the position of basic residue 4) allowed engineering of a Zn²⁺-binding site, which caused Zn²⁺-dependent inhibition of kinase activity, possibly due to distortion of the glycine-rich loop. More importantly, introduction of His in the tail site (T958H) of PRK2-K670H/K689H, generated a Zn²⁺-binding site with increased affinity, as evidenced by more profound inhibition at 3.3 μM and detectable inhibition already at 0.5 μM Zn²⁺. Similar results were obtained after Zn²⁺ site engineering in PKBα (data not shown).

Furthermore, if the tail site Ser/Thr and the basic residues are within interaction distance, mutation of the tail site Ser/Thr to Arg might inhibit kinase activity to a higher extent than an Ala mutation due to electrostatic repulsion of the tail. In MSK1, an S360R mutation indeed inhibited kinase activity to a higher extent (≈90%, FIG. 12B) than an S360A mutation (≈80%, FIG. 10). MSK1-K62E/K84S/K126N, which has an acidic residue in place of basic residue 2 and neutral charge in place of basic residues 3 and 4 had ≈15% activity compared to wt MSK1. Strikingly, in this mutant, introduction of the S360R mutation was not inhibitory, but rather increased kinase activity to ≈40% of that of wt MSK1. In this charge-reversal mutant, R360 presumably binds E62 introduced in place of basic residue 2 and thereby partially rescues kinase activity.

In conclusion, these experiments provide evidence that the tail phosphate and the basic cluster are within interaction distance in the active AGC kinase conformation.

### The Tail Phosphate Synergistically Enhances AGC Kinase Activation by the Phosphorylated HM, Dependent on the Tail Phosphate-Binding Site

We established an in vitro reconstitution assay that could test a direct activation of the AGC kinase domain by the tail phosphate and characterize its cooperation with the HM and activation loop phosphates. The deletion mutant S6K1₁₋₃₆₄, which contains the kinase domain, but lacks the region of the tail containing the tail site and the HM as well as the C-terminal autoinhibitory domain, was expressed and purified from COS7 cells, either non-phosphorylated or phosphorylated at T221 in the activation loop, achieved by co-expression with PDK1. Purified S6K1₁₋₃₆₄ was then incubated with synthetic peptides of the S6K1 tail (residues 366-395: QTPVDS³⁷¹PDDSTLSESANQVFLGFT³⁸⁹YVAPSV), which were either non-phosphorylated (S371/T389), phosphorylated at the tail site (pS371/T389), phosphorylated in the HM (S371/pT389) or phosphorylated at both sites (pS371/pT389). Subsequently, the kinase activity of S6K1₁₋₃₆₄ was determined.

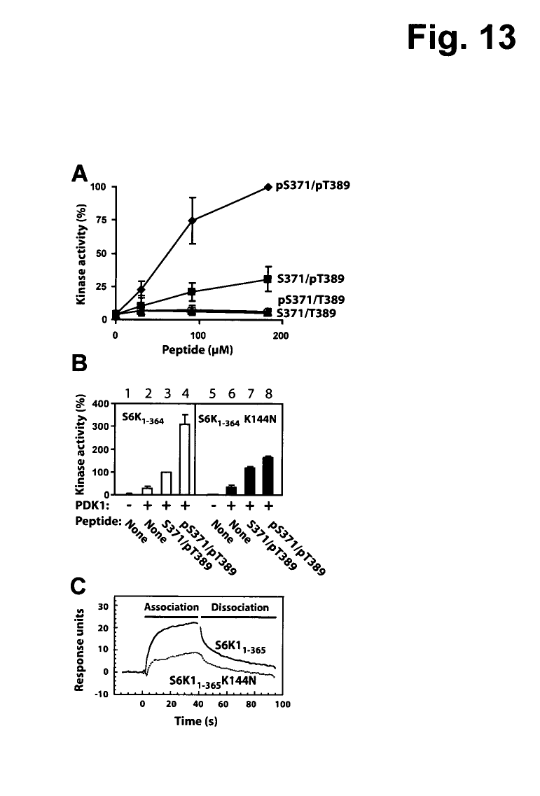

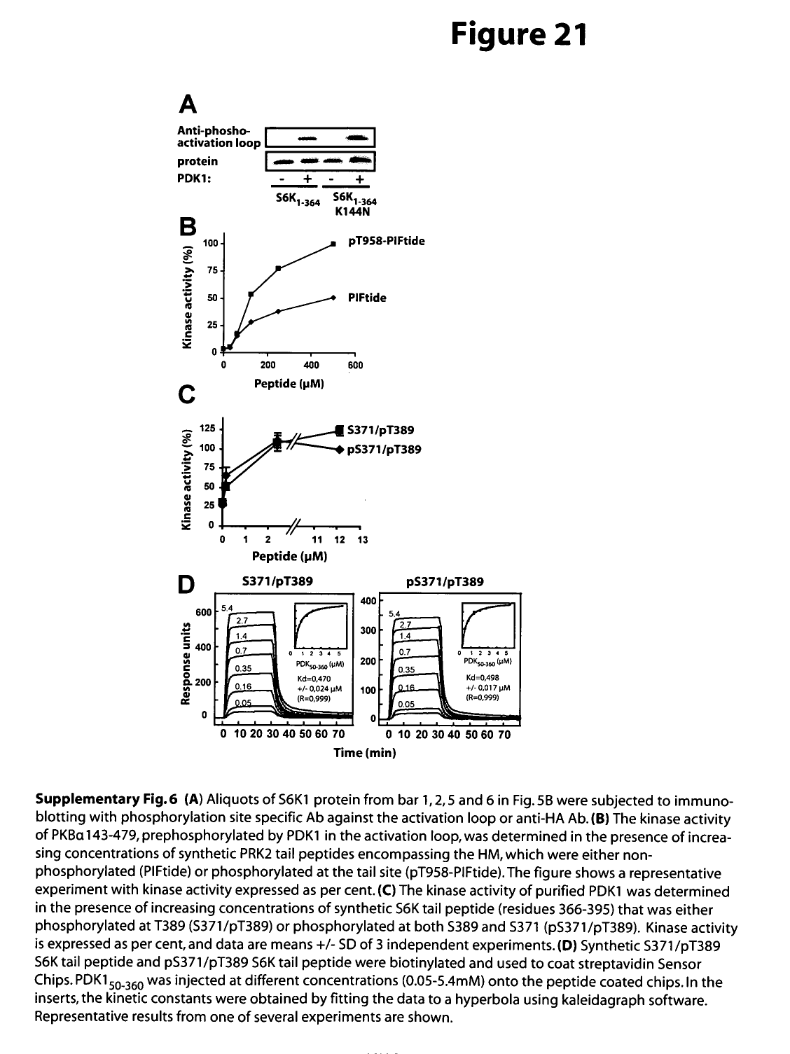

S371/T389 or pS371/T389 tail peptides did not stimulate the kinase activity of Thr221-phosphorylated S6K1₁₋₃₆₄, whereas S371/pT389 peptide induced a 5- to 7-fold stimulation of kinase activity at 190 μM (FIG. 13A). More importantly, pS371/pT389 peptide induced a 16- to 22-fold stimulation of kinase activity at 190 μM. These experiments revealed that the tail phosphate synergistically enhances S6K1 activation by the HM phosphate, while having no effect on its own. In similar experiments with a truncated PKBβ kinase domain, the tail phosphate also enhanced the ability of the HM to stimulate kinase activity (FIG. 21B).

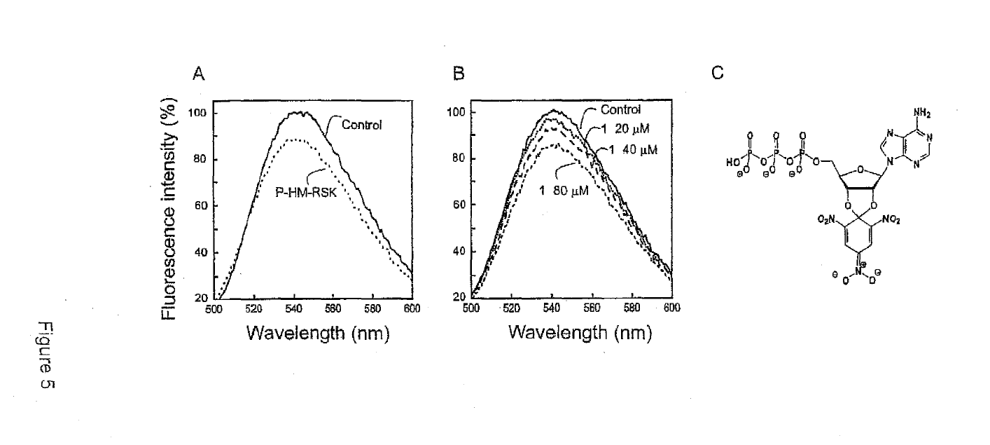

We next investigated the role of the tail phosphate-binding site. The basic residue 4 mutant S6K1₁₋₃₆₄K144N was activated normally by S371/pT389 peptide (FIG. 13B, compare bars 3 and 7), but could not be hyperactivated by pS371/pT389 (compare bars 4 and 8), indicating that the tail phosphate-binding site mediates the kinase-activating effect of the tail phosphate. As a control, S6K1₁₋₃₆₄K144N was phosphorylated and activated normally by PDK1 (compare bars 2 and 6 in FIG. 5B, FIG. 21A), indicating that the K144N mutation did not compromise the tertiary structure of S6K1, but selectively disrupted the tail phosphate-binding site. As another control, PDK1, which lacks a tail phosphate-binding site (FIG. 9B) but contains a pHM-binding site (Biondi et al., 2002; Frodin et al., 2002), was activated to the same extent by S371/pT389 and pS371/pT389 (FIG. 21C).

Surface plasmon resonance measurements revealed that the K144N mutation decreased the binding of S6K1₁₋₃₆₅ to pS371/pT389 peptide by ≈50% (FIG. 13C). Similarly, the K144N mutation decreased the binding of S6K1₁₋₃₆₅ to pS371/T389 peptide by about 40%, whereas the K144N mutation had no effect on the binding of S6K1₁₋₃₆₅ to S371/pT389 peptide (data not shown). No specific binding of S6K1₁₋₃₆₅ or S6K1₁₋₃₆₅K144N to S371/T389 peptide could be detected. Binding constants for these interactions could not be determined using the BiaCore instrument, since the affinities were too low for kinetic analysis, in accordance with the AC₅₀ value of ≈60 μM for pS371/pT389 towards S6K1₁₋₃₆₅ (FIG. 13A). As a control, PDK1 bound equally well to S371/pT389 and pS371/pT389 (FIG. 21D). We conclude that the binding contribution of pS371 in these experiments result from interaction with the tail phosphate-binding site.

These results support the model that the interaction between the tail phosphate and its binding site promotes the binding of the tail to the kinase domain and thereby the tail phosphate synergistically enhances kinase activation by the phosphorylated HM.

The Tail Phosphate Promotes a Compact AGC Kinase Conformation and Protects the Tail Phosphate-Binding Site and αC-Helix from Solvent Exposure as Revealed by Amide Hydrogen (¹H/²H) Exchange and Mass Spectrometry (HXMS)

Proteins analyzed by HXMS are incubated in D₂O and the mass increase resulting from isotopic exchange of backbone amide protons (1 per residue, except for proline) for solvent deuterons is measured by mass spectrometry. Differences in hydrogen exchange rates between protein samples reflect differences in solvent exposure due to conformational change and/or protein-protein interaction. Sites of conformational change/interaction are revealed as regions with increased protection in analyses of peptic digests of the labelled proteins (local HXMS).

Addition of S371/pT389 and pS371/pT389 peptide to S6K1₁₋₃₆₅, resulted in ≈10 and ≈25 fewer deuterons incorporated, respectively, in S6K1₁₋₃₆₅ at early time points of analysis, as compared to no peptide added (FIG. 14A). The non-phosphorylated S371/T389 peptide had no effect, demonstrating that non-specific peptide binding to S6K1₁₋₃₆₅ did not contribute to the surface protection.

We next investigated the effect of the tail phosphate on deuteron uptake in a full-length AGC kinase. For this analysis we chose PKCζ, since only this kinase, among the ones tested, was found to be stoichiometrically phosphorylated at all the regulatory phosphorylation sites, which is critical for HXMS analysis. First, we demonstrated that the tail site T560 is essential for full PKCζ activity and identified basic residues 2 and 4 (K265, K284) as key residues in the tail phosphate-binding site (FIG. 14B). Second, we found that mutation of the tail site and its binding site greatly increased global deuteron uptake by PKCζ and to exactly the same extent (FIG. 14C). At early time points of analysis, the tail phosphate protected ≈60 residues. Finally, we sought to identify specific regions in PKCζ protected by the tail phosphate by local HXMS analysis. Strikingly, peptide (a), corresponding to the regulatory αC-helix was dramatically protected by the tail phosphate (FIG. 14D). Moreover, protection was observed in peptide (b), but not in the overlapping peptide (c). This means that the 2-3 protected residues in peptide (b) are to be found in the sequence AKVLL, which encompasses a C-terminal residue of the glycine-rich loop and basic residue 2 (underlined). Protection was also observed in peptide (d), encompassing part of the αG-helix, consistent with the finding that this helix is disturbed in PKB structures with a disordered αC-helix. Slight protection was observed in peptide (e) corresponding to the start of the tail region. No protection was observed in peptide (f), located in a region of the large lobe not predicted to be affected by the tail phosphate or in peptide (g) located between the tail site and the HM, in agreement with the solvent exposed and flexible nature of this segment, as revealed by its near-complete deuteron uptake in wt PKCζ. For technical reasons, the sequence coverage of PKCζ was not complete and we could therefore not perform a full analysis of the effect of the tail phosphate on local HX.

The local HXMS analysis provides strong evidence that the tail phosphate interacts with basic residue 2 in the binding site and that it promotes a dramatic stabilization of the regulatory αC-helix of the hydrophobic pocket. Moreover, the large extent of protection revealed by global HXMS strongly suggests that the tail phosphate promotes a significant allosteric change to a more compact conformation, which most likely corresponds to the closed, active AGC kinase conformation.

### The Tail Site is not Related to the Turn Motif Site in PKA

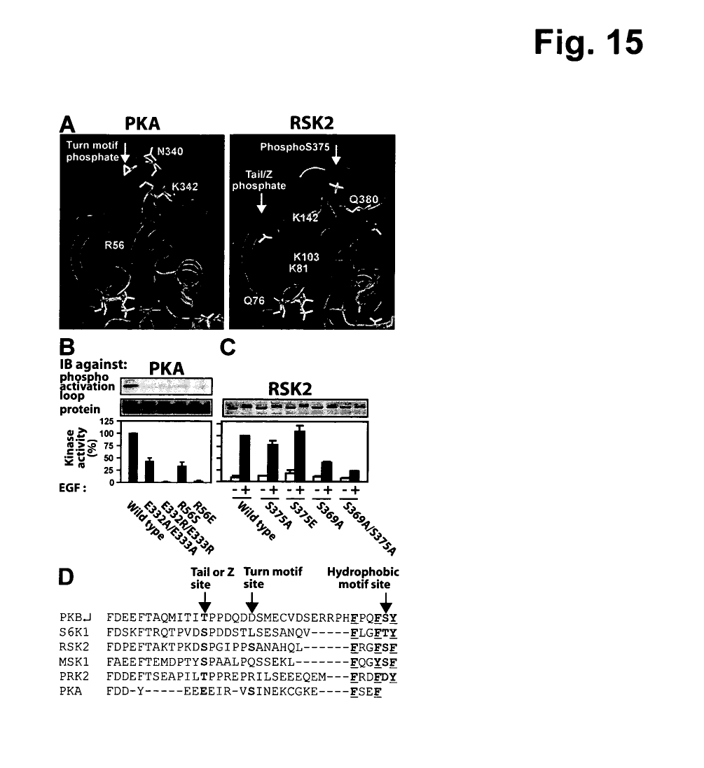

We noticed that in active, but not inactive structures of PKA (1ATP/1YDR/1FMO/1L3R and 1CTP/1CMK, respectively), R56, which aligns with basic residue 2, binds E333 in the PKA tail. Moreover, E333 holds a similar position as the tail phosphate in our models (FIG. 15A). Mutation of R56 or E333/E334 profoundly reduced autophosphorylation in the activation loop and catalytic activity of PKA (FIG. 15B). Individual mutation of E333 had little effect, suggesting compensatory action by E332, which is in close proximity to R56 in active PKA structures. Similar compensatory action has been reported upon mutation of the tail site in PKCβII (Newton, 2001). Our results with PKA suggest that the E333/R56 interaction evolved from the tail phosphate/basic residue 1-4 interaction or vice versa, implying that the tail site should be aligned with E333, rather than with the turn motif site (FIG. 15D). In such a revised alignment, the PKA turn motif site aligns well with S375 in RSK2, recently identified as a site of phosphorylation (Ballif et al., 2005). Interestingly, mutation of S375 to Ala reduced EGF-stimulated RSK2 activity by whereas a phosphate-mimicking Glu mutation did not (FIG. 15C). The inhibitory effects of the S375A and the tail site S369A mutations were additive, indicating that the two phosphorylation sites stimulate RSK2 activity by distinct mechanisms. In agreement with this conclusion, modelling and dynamics simulations suggested that phosphoS375 does not interact with the tail phosphate-binding site but may bind neighbouring polar residues within the tail, thereby resembling the turn motif phosphate in PKA (FIG. 15A).

We conclude that the tail site and the turn motif site are non-related rather than being the same site which adopts two distinct conformations in growth factor-activated AGC kinases and PKA, respectively. Thus, AGC kinases may contain either or both sites, and in PKA, the tail site consists of a phosphate-mimicking Glu. Based on the present findings, we propose to refer to the tail site in growth factor-activated AGC kinases as the Z (zipper) site instead of the turn motif site.

### Discussion

The molecular mechanism whereby the tail phosphorylation site stimulates the activity of the growth factor-activated AGC kinases has been the last unresolved issue in their common activation mechanism. The data presented here support the following model: the tail phosphate interacts with a phosphate-binding site in the small lobe of the kinase domain, located on top of the ATP-binding, glycine-rich loop. The tail phosphate-binding site thereby provides an anchoring point for the tail, which increases the local concentration of the HM in the immediate vicinity of the binding site through which the HM stimulates kinase activity. This increase in local concentration is likely to be important, since the affinity of the phosphorylated HM for its binding site is very low, with estimated K_{d}'s ranging from 30 to 600 μM among various AGC kinases (Frodin et al., 2002; Yang et al., 2002b; this study). By increasing the local concentration, the tail phosphate-binding site enhances the ability of the phosphorylated HM to interact with the hydrophobic pocket. Our model further proposes that the tail phosphate-binding site thereby allosterically affects the αC-helix and protects the HM from dephosphorylation.

The tail phosphate-binding site thus promotes kinase activity by at least two mechanisms. In the first mechanism, which likely operates in all of the growth factor-activated AGC kinases, the tail phosphate-binding site allosterically supports the re-ordering of the HM-binding pocket, including the αC-helix. Stabilization of the αC-helix is thought to be of key importance in activation of AGC kinases. In the ordered αC-helix, a conserved Glu stabilizes a conserved Lys, which positions the α- and β-phosphates of ATP, whereas other residues are thought to stabilize the activation loop, which likely constitutes a mechanism for cooperation between the HM and activation loop phosphates in stimulation of kinase activity. It is therefore a highlight of the present study that local HXMS analysis showed dramatic stabilization of the αC-helix by the tail phosphate. We estimate that the tail phosphate protects ≈50% of the residues in the αC-helix, providing strong evidence for our model that the tail phosphate functions to aid the HM to bind and re-order the hydrophobic pocket. The results also provide the first in-solution evidence of stabilization of the αC-helix during AGC kinase activation. Previously, the disorder-to-order transition of the αC-helix was supported mainly by comparison of crystal structures of inactive and active PKB (Yang et al., 2002a; Yang et al., 2002b). Global HXMS analysis suggested that ≈60 residues were protected by the tail phosphate in PKCζ, a number exceeding the residues in the tail phosphate-binding sites and the αC-helix. This suggests that the tail phosphate promotes a significant allosteric change, which we assume corresponds to stabilization also of the αB-helix, another component of the HM-binding pocket and of the activation loop, leading to stabilization of the entire kinase domain in the closed, active conformation.

In the second mechanism, the tail phosphate-binding site stimulates kinase activity by increasing the phosphorylation level in the HM. This mechanism operates only in a subset of the AGC kinases such as S6K, MSK and to a slight extend RSK (this study), and likely also in several PKCs, where mutation of the tail site decreases HM phosphorylation (Parekh et al., 2000; Newton, 2003). The second mechanism is most likely linked to the first mechanism, since interaction of the phosphorylated HM with its binding site presumably renders the phosphate less accessible to phosphatases due to its binding to several charged/polar residues. The reason that the second mechanism may operate only in some AGC kinases may partly be due to the possibility that the HM in the various AGC kinases is targeted by distinct phosphatases with varying efficiency.

Basic residues 1 and 2 are located at the base of the glycine-rich loop which positions the γ-phosphate of ATP for phosphotransfer. In our HXMS analysis, the tail phosphate affected the flexibility of the segment AKVLL which encompasses part of the glycine-rich loop and basic residue 2. It would be interesting to investigate whether the tail phosphate may promote kinase activity by modulating the position of the glycine-rich loop via basic residue 2 in addition to the two mechanisms described above.

Our data suggest that the tail phosphate-binding site is composed of 4 basic residues. The in-dispensability of individual basic residues varied among the kinases. For instance, in S6K1 and PRK2, individual mutation of basic residue 4 and 1 to 3, respectively, inhibited kinase activity by >85%, whereas in PKBα and RSK2, all 4 basic residues must be mutated to achieve substantial inhibition. In some kinases we observed that individual mutation of a particular basic residue had negligible effect, but the same mutation enhanced the inhibitory effect of other basic residue mutations (data not shown). However, caution needs to be taken regarding conclusions on the exact contribution/importance of specific basic residues, since we introduced semi-conservative substitutions like Ser, Thr or Asn with some phosphate-binding ability. Nevertheless, taken together, our results suggest that the tail phosphate may bind several distinct combinations of basic residues, which may change dynamically over time. This possibility is supported by the open appearance of the tail phosphate-binding site in the PKBβ crystal structure and by our molecular dynamics simulations. Similar dynamic interaction modes may also exist for the PKA turn motif phosphate, perhaps due to the flexible nature of the PKA tail region (Johnson et al., 2001). Thus, the turn motif phosphate binds R336, N340 and K342 in the structure 1FMO, N340 and K342 in 1ATP, and none of these residues in 1BKK. In the growth factor-activated AGC kinases, the 4 basic residues might perform partly distinct roles. For instance, basic residue 1 may function mainly to attract the tail phosphate, followed by docking of the tail phosphate to the more deeply positioned basic residues 2 to 4. Basic residues 3 and 4 are located in the non-flexible β-strands 3 and 5, respectively. Binding to these residues may fix the tail phosphate, allowing it to affect the position of the non-rigid glycine-rich loop via interaction with basic residue 2.

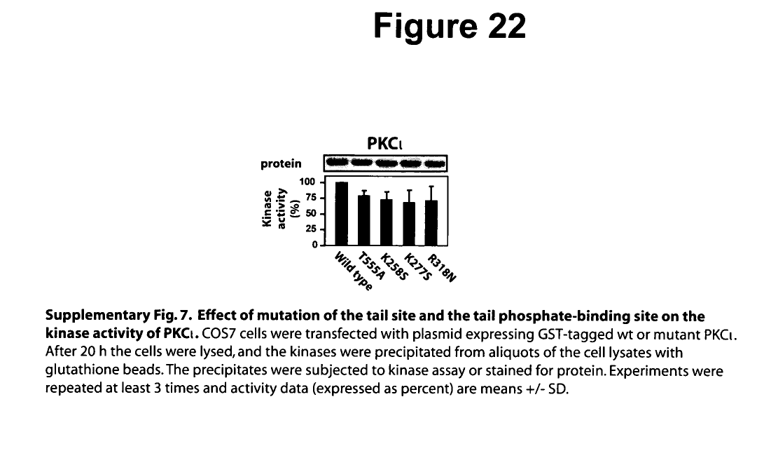

Recently, a crystal structure of the PKCt kinase domain was reported (Messerschmidt et al., 2005). In this structure, the tail phosphate binds basic residues 3 and 4, in agreement with our study. Unlike our model, the authors speculated that the phosphate may function 1) to stabilize the kinase domain, referring to the destabilizing effect of mutation of the turn motif site of PKA and the kinase-inactivating effect of mutation of the tail site in PKC, 2) to push the tail out of the active site or 3) to regulate the interaction of the kinase domain with the flanking signalling modules in PKC. The role of the tail phosphate and its interactions were not investigated. The side chain of the basic residue 2 Lys was not visible in the structure, but we noted that its backbone is located immediately beneath the tail phosphate, allowing binding of the side chain to the tail phosphate. We therefore mutated the tail site T555 and the basic residues 2-4 and observed a ≈25% reduction of kinase activity (FIG. 22). The mutational analysis thus revealed that the interaction is required for full PKCι activation, but also showed that the tail phosphate is less important for PKCι than for other AGC kinases that we have analyzed.

The present study provides the first characterization of the cooperation between all 3 conserved phosphates in stimulation of AGC kinase activity by using an in vitro reconstitution assay based on long tail peptides. Our data showed that the 3 phosphates act in a hierarchal manner: the tail phosphate has no activating effect alone or together with the activation loop phosphate. However, the tail phosphate synergistically enhances the ability of the HM phosphate to stimulate kinase activity in cooperation with the activation loop phosphate. Global HXMS analysis in this system suggested that the tail phosphate promoted a significant allosteric change, which we assume represents HM-mediated transition to the closed, active AGC kinase conformation. Our model and data thus implies that the activating conformational change induced by binding of the HM to the hydrophobic pocket is triggered not only by HM phosphorylation, as previously suggested, but is regulated by the dual phosphorylation events in the tail site and the HM, working in a cooperative manner.

These results together with previous studies underscore the extensive cooperation of the 3 conserved phosphorylation sites with respect to shifting the equilibrium of the AGC kinase catalytic domain from the inactive, open conformation towards the active, closed conformation during stimulus-induced activation. This cooperativity is likely also an important determinant during inactivation. In many of the kinases, the phosphorylation sites are rapidly dephosphorylated upon cessation of the activating stimulus. In this process, dephosphorylation at one site may greatly decrease the ability of the remaining phosphates to support the active, closed and more phosphatase-resistant conformation, resulting in accelerated dephosphorylation of the remaining phosphates and complete inactivation of the kinase.

Elevated PKB activity is essential for the progression of many human cancers and may result from mutations in e.g. PI3-K or PTEN. Recently, 2 point mutations in PKB were identified in colorectal cancer (Parsons et al., 2005). The present study reports mutations in the tail phosphate-binding site which yielded a considerable degree of activation and which stimulated cell growth (C. Hauge and M. Frodin, unpublished observation). Activation appeared to result from increased phosphorylation in the HM and the activation loop. Presumably, the aberrantly exposed HM of PKB in these mutants becomes hyperphosphorylated by the physiological PKB HM kinase or by some other HM kinase. Profound hyperphosphorylation of PKB was not observed after severe disruption of zipper function such as in PKBα-T450A. Conceivably, if the HM of PKB becomes excessively exposed, phosphatase action counteracts kinase action. The increased activation loop phosphorylation may be a consequence of the increased HM phosphorylation, since the phosphorylated HM is thought to promote the closed, and more phosphatase-resistant conformation of the AGC kinase domain. Alternatively, the abnormally exposed HM may increase activation loop phosphorylation by recruitment of PDK1, as HM phosphorylation may enhance phosphorylation of PKB by PDK1 under certain conditions (Scheid et al., 2002). Regardless of the mechanism, our results suggest that elevated PKB activity in cancer cells may result from mutations in the tail phosphate-binding site of PKB.

Our study suggests that the tail site and the turn motif site are two distinct phosphorylation sites that should not be aligned. Since it is problematic to use the designation “turn motif” for two distinct sites, we propose to refer to the tail site as the Z (zipper) site, a name which reflects one major function of this phosphorylation site according to the present findings.

In conclusion, the present study provides important information on the missing pieces in the core mechanism, whereby 3 conserved phosphorylation sites stabilize the active conformation of up to 26 human AGC kinases, which thus represents one of the most general activation mechanisms reported in the human kinome.

### REFERENCES

Alessi, D. R., Andjelkovic, M., Caudwell, B., Cron, P., Morrice, N., Cohen, P. and Hemmings, B. A. (1996) Mechanism of activation of protein kinase B by insulin and IGF-1. Embo J, 15, 6541-6551.
Ballif, B. A., Roux, P. P., Gerber, S. A., MacKeigan, J. P., Blenis, J. and Gygi, S. P. (2005) Quantitative phosphorylation profiling of the ERK/p90 ribosomal S6 kinase-signaling cassette and its targets, the tuberous sclerosis tumor suppressors. Proc Natl Acad Sci USA, 102, 667-672.
Bellacosa, A., Chan, T. O., Ahmed, N. N., Datta, K., Malstrom, S., Stokoe, D., McCormick, F., Feng, J. and Tsichlis, P. (1998) Akt activation by growth factors is a multiple-step process: the role of the PH domain. Oncogene, 17, 313-325.
Biondi, R. M., Cheung, P. C., Casamayor, A., Deak, M., Currie, R. A. and Alessi, D. R. (2000) Identification of a pocket in the PDK1 kinase domain that interacts with PIF and the C-terminal residues of PKA. Embo J., 19, 979-988.
Biondi, R. M., Kieloch, A., Currie, R. A., Deak, M. and Alessi, D. R. (2001) The PIF-binding pocket in PDK1 is essential for activation of S6K and SGK, but not PKB. Embo J., 20, 4380-4390.
Biondi, R. M., Komander, D., Thomas, C. C., Lizcano, J. M., Deak, M., Alessi, D. R. and van Aalten, D. M. (2002) High resolution crystal structure of the human PDK1 catalytic domain defines the regulatory phosphopeptide docking site. Embo J, 21, 4219-4228.
Dalby, K. N., Morrice, N., Caudwell, F. B., Avruch, J. and Cohen, P. (1998) Identification of Regulatory Phosphorylation Sites in Mitogen-activated Protein Kinase (MAPK)-activated Protein Kinase-1a/p90rsk That Are Inducible by MAPK. J. Biol. Chem., 273, 1496-1505.
Frodin, M., Antal, T. L., Dummler, B. A., Jensen, C. J., Deak, M., Gammeltoft, S. and Biondi, R. M. (2002) A phosphoserine/threonine-binding pocket in AGC kinases and PDK1 mediates activation by hydrophobic motif phosphorylation. Embo J., 21, 5396-5407.
Frodin, M., Jensen, C. J., Merienne, K. and Gammeltoft, S. (2000) A phosphoserine-regulated docking site in the protein kinase RSK2 that recruits and activates PDK1. Embo J., 19, 2924-2934.
Hauge, C. and Frodin, M. (2006) RSK and MSK in MAP kinase signalling. J Cell Sci, 119, 3021-3023.
Johnson, D. A., Akamine, P., Radzio-Andzelm, E., Madhusudan, M. and Taylor, S. S. (2001) Dynamics of cAMP-dependent protein kinase. Chem Rev, 101, 2243-2270.
Knighton, D. R., Zheng, J. H., Ten Eyck, L. F., Ashford, V. A., Xuong, N. H., Taylor, S. S. and Sowadski, J. M. (1991) Crystal structure of the catalytic subunit of cyclic adenosine monophosphate-dependent protein kinase. Science, 253, 407-414.
Kobayashi, T., Deak, M., Morrice, N. and Cohen, P. (1999) Characterization of the structure and regulation of two novel isoforms of serum- and glucocorticoid-induced protein kinase. Biochem J, 344 Pt 1, 189-197.
Kozma, S. C. and Thomas, G. (2002) Regulation of cell size in growth, development and human disease: PI3K, PKB and S6K. Bioessays, 24, 65-71.
Matsuzaki, H., Yamamoto, T. and Kikkawa, U. (2004) Distinct activation mechanisms of protein kinase B by growth-factor stimulation and heat-shock treatment. Biochemistry, 43, 4284-4293.
McCoy, C. E., Campbell, D. G., Deak, M., Bloomberg, G. B. and Arthur, J. S. (2004) MSK1 activity is controlled by multiple phosphorylation sites. Biochem J.
Messerschmidt, A., Macieira, S., Velarde, M., Badeker, M., Benda, C., Jestel, A., Brandstetter, H., Neuefeind, T. and Blaesse, M. (2005) Crystal structure of the catalytic domain of human atypical protein kinase C-iota reveals interaction mode of phosphorylation site in turn motif. J Mol Biol, 352, 918-931.
Moser, B., Dennis, P., Pullen, N., Pearson, R., Williamson, N., Wettenhall, R., Kozma, S, and Thomas, G. (1997) Dual requirement for a newly identified phosphorylation site in p70s6k. Mol. Cell. Biol., 17, 5648-5655.
Newton, A. C. (2001) Protein kinase C: structural and spatial regulation by phosphorylation, cofactors, and macromolecular interactions. Chem Rev, 101, 2353-2364.
Newton, A. C. (2003) Regulation of the ABC kinases by phosphorylation: protein kinase C as a paradigm. Biochem J, 370, 361-371.
Parekh, D. B., Ziegler, W. and Parker, P. J. (2000) Multiple pathways control protein kinase C phosphorylation. Embo J, 19, 496-503.
Parsons, D. W., Wang, T. L., Samuels, Y., Bardelli, A., Cummins, J. M., DeLong, L., Silliman, N., Ptak, J., Szabo, S., Willson, J. K., Markowitz, S., Kinzler, K. W., Vogelstein, B., Lengauer, C. and Velculescu, V. E. (2005) Colorectal cancer: mutations in a signalling pathway. Nature, 436, 792.
Roux, P. P. and Blenis, J. (2004) ERK and p38 MAPK-activated protein kinases: a family of protein kinases with diverse biological functions. Microbiol Mol Biol Rev, 68, 320-344.
Saitoh, M., Pullen, N., Brennan, P., Cantrell, D., Dennis, P. B. and Thomas, G. (2002) Regulation of an Activated S6 Kinase 1 Variant Reveals a Novel Mammalian Target of Rapamycin Phosphorylation Site. J. Biol. Chem., 277, 20104-20112.
Scheid, M. P., Marignani, P. A. and Woodgett, J. R. (2002) Multiple phosphoinositide 3-kinase-dependent steps in activation of protein kinase B. Mol Cell Biol, 22, 6247-6260.
Smith, K. J., Carter, P. S., Bridges, A., Horrocks, P., Lewis, C., Pettman, G., Clarke, A., Brown, M., Hughes, J., Wilkinson, M., Bax, B. and Reith, A. (2004) The structure of MSK1 reveals a novel autoinhibitory conformation for a dual kinase protein. Structure (Camb), 12, 1067-1077.
Weng, Q.-P., Kozlowski, M., Belham, C., Zhang, A., Comb, M. J. and Avruch, J. (1998) Regulation of the p70 S6 Kinase by Phosphorylation in Vivo. ANALYSIS USING SITE-SPECIFIC ANTI-PHOSPHOPEPTIDE ANTIBODIES. J. Biol. Chem., 273, 16621-16629.
Yang, J., Cron, P., Good, V. M., Thompson, V., Hemmings, B. A. and Barford, D. (2002a) Crystal structure of an activated Akt/protein kinase B ternary complex with GSK3-peptide and AMP-PNP. Nat Struct Biol, 9, 940-944.
Yang, J., Cron, P., Thompson, V., Good, V. M., Hess, D., Hemmings, B. A. and Barford, D. (2002b) Molecular mechanism for the regulation of protein kinase B/Akt by hydrophobic motif phosphorylation. Mol Cell, 9, 1227-1240.

### Materials and Methods

### Antibodies

Anti-p5386 RSK1 Ab (#06-826), detecting the phosphorylated HM of RSK, and anti-pS363 RSK1 Ab (#06-824), detecting the phosphorylated tail site of RSK, were from Upstate Biotechnology. Anti-pT389 S6K1 Ab (#9205), detecting the phosphorylated HM of S6K, anti-pS371 S6K1 Ab (#9208), detecting the phosphorylated tail site of S6K, anti-pT308 PKB Ab (#9275), detecting the phosphorylated activation loop of PKB, anti-pS360 MSK1 Ab (#9594), detecting the phosphorylated tail site of MSK, and anti pS376-MSK1 Ab (#9591), detecting the phosphorylated HM of MSK, were from Cell Signaling Technology. Anti-pS227 RSK2 Ab (#sc-12445-R), detecting the phosphorylated activation loop of RSK, anti-pS473 PKB Ab (#sc-7985-R), detecting the phosphorylated HM of PKB, anti-pT256 SGK Ab (#sc-16744-R), detecting the phosphorylated activation loop of S6K, PRK, and PKA, anti-PKA Ab (#sc-903), and rabbit anti-HA Ab (#sc-805) for immunoblotting were from Santa Cruz Biotechnologies. Anti-pT641 PKCβ Ab (#GTX25785), detecting the phosphorylated tail site of PRK and PKB, were from GeneTex, Inc. Anti-HA Ab for immunoprecipitation was from the 12CA5 mouse hybridoma cell line.

### Plasmid Constructs

pMT2-HA-RSK2 (mouse) (Zhao et al., 1996) was kindly provided by Dr. Christian Bjørbæk (Beth Israel Hospital, Boston, Mass., USA). pECE-PKBα-HA (human) and pRK5-GST-myc-S6K1 (the rat 70 kDa splice variant) are described in Kohn et al. (1995) and Pullen et al. (1998), respectively. pCMV5-myc-PDK1 (human) is described in Jensen et al. (1999). pMT2-HA-MSK1 (human) is described in Frodin et al. (2000). PRK2_{Δ1-500} (human) and pEBG-2T-PKCzeta (human) are described in Balendran et al. (2000). pMT2-HA-S6K1₁₋₃₆₄ (rat) is described in Frodin et al. (2002). pEBG-2T-GST-S6K₁₋₃₆₅ (rat) was generated by PCR amplification of the desired p70 S6K1 sequence using a 5′ primer introducing a BamHI site and a 3′ primer introducing a stop codon and a KpnI site. The PCR product was inserted in pEBG-2T vector digested with BamHI and KpnI. pEBG-2T-PKB S473D (human) and pEBG-2T-ΔPH-PKB S473D (human) are described in Biondi et al. (2001). PKCι (human) was cloned into the pEBG-2T vector. pT7-7-PKA was kindly provided by Dr. Dirk Bossemeyer (German Cancer Research Centre, Heidelberg, Germany). pLNCX-HA-Akt1 was kindly provided by William Sellers (Addgene plasmid 9004) and is described in Ramaswamy et al. (1999). Point mutations were introduced using the QuickChange mutagenesis procedure (Stratagene) and confirmed by sequencing.

### Modelling Procedures

Potential phosphate-binding sites in the PKBβ structure PDB entry 1OK6 (Yang et al., 2002a) were identified using GRID, version 2.2, (Goodford, 1985) and GRASP (http://trantor.bioc.columbia.edu/grasp/) which can compute energetically favourable binding sites for phosphate groups and electrostatic surface potentials of proteins, respectively. In 1OK6, non-visible tail up until the tail phosphorylation site was ab initio modelled using the Builder module in Insight II (version 2005) from Accelrys. The tail region between the tail site and the HM was generated with the random tweak algorithm in the Homology module in Insight II. The kinase domain and tail/linker region of mouse RSK2 (residues 62-387) and rat S6K1 (residues 62-390) were homology modelled using the visible regions of 1OK6 as a template. The sequence identity of PKBβ and RSK2 or S6K1 is 45% and 51%, respectively. The tail region not visible in 1O6K was modelled as described above. Fully constrained models were then surrounded by a box of water and subjected to dynamics simulation for 10 ps. Thereafter, unconstrained models in water were subjected to dynamics simulation for 10 ps. Before all dynamics simulations, energy minimization of the models was performed with 500 iterations using AMBER force field and the steepest descents algorithm in Insight II. The program PROCHECK (Laskowski et al., 1993) was used to validate the geometry of the models obtained after dynamics simulation.

### Cell Culture

COS7 cells were cultured at 37° C., in atmospheric air containing 5% CO₂, in Dulbecco's modified Eagle's medium with GlutaMAX (Gibco, #31966) supplemented with 10% foetal bovine serum, 100 U/ml penicillin and 100 μg/ml streptomycin. Drosophila S2 cells were cultured at 25° C. in Schneiders' Drosophila medium with L-glutamine (Gibco, #21720) supplemented with 10% foetal bovine serum, 100 U/ml penicillin, and 100 μg/ml streptomycin.

### Transfection, Immunoprecipitation and Protein Staining

COS7 cells were transfected using Lipofectamine 2000 reagent (Life Technologies, Inc.) or using FuGENE 6 reagent (Roche) according to the manufacturers instructions, using 4 μg DNA to 11 μl Lipofectamine 2000 or 1 μg DNA to 3 μl FuGENE 6, respectively, per 9.6 cm² dish. Drosophila S2 cells were transfected with FuGENE HD reagent (Roche) according to the manufacturers instructions, using 2 μg DNA to 4 μl FuGENE HD per 9.6 cm² dish. Cells were harvested the following day (COS7 cells) or after 2 days (Drosophila S2 cells) by solubilization for 15 min in 500 μl lysis buffer (0.5% Triton X-100, 150 mM NaCl, 50 mM Tris-HCl (pH 7.5), 1 mM Na₃VO₄, 5 mM EDTA, 50 mM NaF, 10 nM calyculin A, 10 μM leupeptin, 5 μM pepstatin, 1 μg/ml aprotinin) on ice and manipulated at <4° C. thereafter. For kinase assays with S6K or PRK2, 1 mM dithiothreitol was added to the lysis buffer. Cell extracts were clarified by centrifugation for 5 min at 18,000 g and the supernatant was incubated for 90 min with antibody with the addition of protein G agarose beads (Upstate) during the final 30 min or with glutathione-Sepharose beads (Amersham Pharmacia Biotech) for 60 min. The beads were then precipitated by centrifugation, washed 5 times with lysis buffer, drained and dissolved in SDS-PAGE sample buffer (2% sodium dodecyl sulfate, 62 mM Tris-HCl (pH 6.8), 10% glycerol, 50 mM dithiothreitol, 0.12% bromophenol blue). For kinase assays, the final 2 washes were with buffer A (30 mM Tris-HCl (pH 7.5), 10 mM MgCl₂, 1 mM dithiothreitol). Kinase protein amounts in precipitates were evaluated by subjecting the precipitate to SDS-PAGE and staining of the gel with SimplyBlue™ protein staining (Invitrogen) or immunoblotting with anti-HA Ab.

### Immuno Complex Kinase Assays

Kinase assays for PKB, RSK, S6K, MSK were done as described previously (Frodin et al., 2002). Agarose beads with precipitated kinase were resuspended in 20 μl 1.5× buffer A. The kinase reaction was initiated by the addition of 10 μl (final concentrations) 100 μM ATP (0.5 μCi [γ-³²P]ATP), and 130 μM S6 peptide (RRLSSLRA; in S6K and RSK assays), or 143 μM Crosstide (GRPRTSSFAEG; in PKB and MSK assays), or 8 μM myelin basic protein (in PRK2 assays). In PKCι and PKCζ assays 40 μM PKCζ substrate peptide (SIYRRGSRRWRKL from Calbiochem) was used. The specific activities of PKCι and PKCζ in these assays were similar to or higher than the specific activities of e.g. S6K and PKB using the well-established assays for these kinases. In Zn²⁺ site engineering experiments, Zn²⁺ was added to the precipitated kinases 5 min before the kinase assay was initiated. After 10 min of kinase reaction at 30° C. with vigorous shaking (the reaction was linear with time in all of the assays), 20 μl of the supernatant were removed and spotted onto phosphocellulose paper (Whatman p81). After washing with 150 mM orthophosphoric acid, [³²P]phosphate incorporated into peptide substrate was quantified in an FLA-3000 apparatus (Fujifilm).

### Immunoblotting

Immunoblotting of precipitated kinases were performed as described (Jensen et al., 1999) but with the following modifications. Hybond-ECL nitrocellulose membranes (Amersham Biosciences) were used, and for immunoblots with the phosphospecific anti-pS360 MSK1 Ab, anti-pS376 MSK1 Ab and anti-pS363 RSK1 Abs, Baileys Irish liquor were used for blocking, as this proved superior in blocking background staining with these Ab compared to skimmed milk, which was used for all other immunoblots.

### PKA Expression and Kinase Assay

Expression and kinase assay of PKA were done exactly as described (Batkin et al., 2000) with minor modifications. Specifically, we used Rosetta™ 2(DE3) instead of BL21(DE3) E. coli for expression, and the lysis buffer composition was slightly different. Rosetta™ 2(DE3) (Novagen #71400) was transformed with pT7-7-PKA expression constructs. Fifty ml LB medium containing 50 μg/ml ampicillin and 37 μg/ml chloramphenicol were inoculated with 5 ml of a stationary phase culture of transformed Rosetta™ 2(DE3), and the culture was grown at 37° C. with vigorous shaking until an absorbance of 0.4-0.6 at 600 nm was reached. Protein expression was then induced by addition of 0.5 mM isopropyl-β-D-thiogalactopyranoside, and allowed to proceed for 4 hours. The bacteria were collected by centrifugation, resuspended in 32 ml buffer (20 mM Tris-HCl (pH 7.4), 1.5 mM MgCl₂, 1 mM dithiothreitol, and 0.2% Triton X-100), and lysed in an ultrasound disintegrator. Insoluble material was removed by centrifugation at 25.000 g for 45 min at 4° C., and the supernatant was snap frozen in liquid N₂ and stored at −80° C. Equal expression of wt and mutant PKA was verified by immunoprecipitation with anti-PKA Ab, followed by SDS-PAGE and protein staining. For kinase assays, the supernatant was diluted appropriately (typically ≈8-times), in a total of 20 μl 1.5× buffer A (30 mM Tris-HCl (pH 7.5), 10 mM MgCl₂, 1 mM dithiothreitol). The kinase reaction was initiated by the addition of 10 μl (final concentrations) 100 μM ATP (0.5 μCi [γ-³²P]ATP), and 108 μM Kemptide (LRRASLG from Calbiochem). After 10 min at 30° C. with vigorous shaking (the reaction was linear with time) the kinase reaction was stopped and quantified as described under “Immuno complex kinase assays”.

### S6K1 and PKB In Vitro Reconstitution Kinase Assay

HA-56K₁₋₃₆₄ (rat) co-expressed with myc-PDK1 (at the plasmid ratio 3:1) in COS7 cells was immunoprecipitated as described above. Human PKBβ₁₄₃₋₄₇₉ (i.e. a deletion mutant lacking the PH domain) was expressed in insect cells as a His-tagged protein and purified by Ni-NTA Sepharose chromatography, followed by cleavage of the His-tag with TEV protease, re-purification through Ni-NTA Sepharose and gel filtration. The protein was essentially pure as estimated by SDS-PAGE and IEF. PKBβ₁₄₃₋₄₇₉ was then phosphorylated in vitro in the activation loop site (T309) by incubation with GST-PDK1, followed by removal of GST-PDK1 by precipitation with glutathione-Sepharose beads. Purified HA-S6K₁₋₃₆₄ was incubated with synthetic S6K1 (rat) tail peptides (residues 366-395) 5 min prior to kinase assays, and purified PKBβ₁₄₃₋₄₇₉ was incubated with the synthetic peptide PIFtide (REPRILSEEEQEMFRDFDYIADWC), or pT958-PIFtide, a synthetic extended PIFtide phosphorylated at the tail site Thr958 (ILT⁹⁵⁸PPREPRILSEEEQEMFRDFDYIADW) 5 min prior to kinase assay. S6K1 (rat) tail peptides were >98% pure and synthesized by Pepceuticals Limited. They contained 3 extra C-terminal residues (EEK) not present in S6K1, that were added to allow C-terminal biotinylation for surface plasmon resonance measurements.

### Purification of GST-S6K₁₋₃₆₅ and GST-PKC; Protein for Surface Plasmon Resonance Measurements and Hydrogen Exchange Analysis.

Sixty 146 cm² dishes with COST cells were co-transfected with pEBG-2T-S6K₁₋₃₆₅ and pCMV5-myc-PDK1 (ratio 3:1) using calcium phosphate precipitation or with pEBG-2T-PKCζ and pCMV5-myc-PDK1 (ratio 3:1) using Fugene HD according to the manufacturers Instructions. Two days after transfection, the cells were harvested in lysis buffer as described above. Cell lysates were incubated with glutathione-Sepharose beads for 60 min at 4° C. Beads were then precipitated by centrifugation, washed 6 times with a high salt lysis buffer (0.5% Triton X-100, 500 mM NaCl, 50 mM Tris-HCl (pH 7.4)) and 3 times with elution buffer (10 mM HEPES (pH 7.4), 150 mM NaCl, 0.005% tween-20 for surface plasmon resonance measurements or 25 mM Tris-HCl (pH 7.4), 150 mM NaCl, 1 mM dithiothreitol for hydrogen exchange analysis) and then drained. The proteins were then eluted with 25 mM glutathione and incubated for 10 min at room temperature with gentle shaking. The eluted protein comprised ˜60% GST-fusion protein, ˜40% free GST and few detectable protein impurities (<5%) as estimated by SDS-PAGE and protein staining. To remove free GST from GST-PKCζ fusion protein for local hydrogen exchange analysis, GST-PKCζ was purified by gel filtration on a Superose 12 10/300 GL column (GE Healtcare #17-5173-01), followed by upconcentration of GST-PKCζ on a Centricon MW50 column. The purified GST-PKCζ was >95% pure as estimated by SDS-PAGE and protein staining.

### Surface Plasmon Resonance Measurements.

BiaCore analysis was performed as described in Biondi et al. (2000). Biotinylation of S6K1 tail peptides was performed as described in Frodin et al. (2002).

Amide hydrogen (¹H/²H) exchange monitored by mass spectrometry (HXMS). D₂O (99.9 atom % D) was obtained from Cambridge Isotope Laboratories. Isotopic exchange was initiated by diluting 8 μl 20-40 μM protein in 300 μl deuterated buffer (25 mM Tris, 150 mM NaCl, 1 mM dithiothreitol, 10 mM MgCl₂, 25 mM glutathione, pD 7.1 uncorrected value). The exchange was carried out at 30° C., and 75 μl aliquots were collected after 1 min, 5 min, 15 min, and 45 min. Exchange was quenched by adding 5 μl quench solution (10% TFA, 8 M guanidine chloride) and snap-frozen in liquid nitrogen where samples were stored until further analysis. As a non-deuterated sample, 2 μl 20-40 μM protein was diluted in 5 μl quench solution and 75 μl water. In analysis of S6K1₁₋₃₆₅ and S6K tail peptides, S6K₁₋₃₆₅ was preincubated with peptides in a 150 molar excess on ice prior to isotopic exchange.

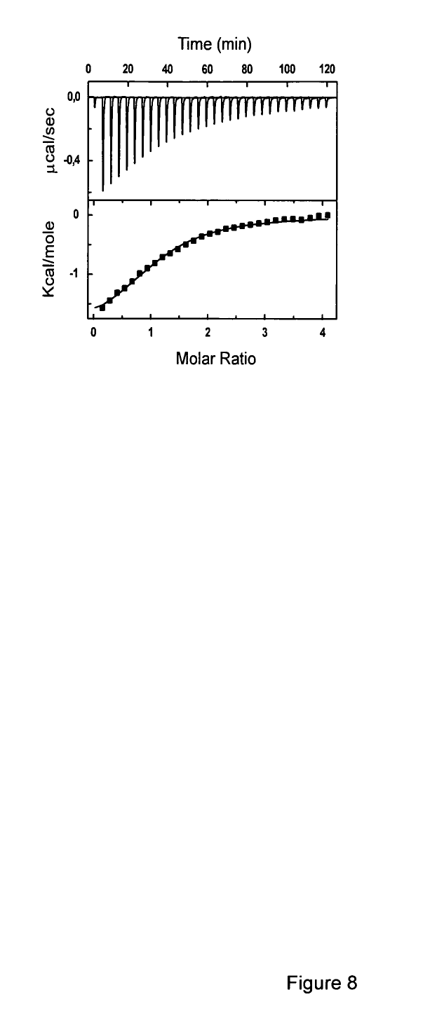

The LC setup was described previously (Jorgensen et al., 2004), with the modification that proteins were desalted for 5 min and eluted with a 9 min linear gradient (14% to 70% acetonitrile, 0.05% TFA) to separate the free GST from the GST-tagged kinases of interest in the global hydrogen exchange analysis. The LC system was coupled to an electrospray ionization quadrupole time-of-flight (QToF Ultima, Micromass) mass spectrometer. Spray voltage was 3.5 kV, cone voltage 55 V, RF lens 1 voltage 100 V, and ion source block temperature 120° C. with a desolvation gas flow of 500 l/h at 200° C. and nebulizing gas flow of 20 l/h at room temperature. The protein charge state envelope was deconvoluted by the MaxEnt 1 algorithm provided with the Masslynx software. To monitor the hydrogen exchange at the peptide level, the protein was digested with pepsin by replacing the injection loop with a column with immobilized pepsin. The protein was digested for 1 min and resulting peptides were desalted and eluted as described above. The HXMS data on peptide level were analyzed by HX-Express (Weis et al., 2006). Examples of the primary data are shown in Supplementary FIG. 8.

### MATERIALS AND METHODS REFERENCES

Balendran, A., Biondi, R. M., Cheung, P. C., Casamayor, A., Deak, M. and Alessi, D. R. (2000) A 3-phosphoinositide-dependent protein kinase-1 (PDK1) docking site is required for the phosphorylation of protein kinase Czeta (PKCzeta) and PKC-related kinase 2 by PDK1. J Biol Chem, 275, 20806-20813.
Batkin, M., Schvartz, I. and Shaltiel, S. (2000) Snapping of the carboxyl terminal tail of the catalytic subunit of PKA onto its core: characterization of the sites by mutagenesis. Biochemistry, 39, 5366-5373.
Biondi, R. M., Cheung, P. C., Casamayor, A., Deak, M., Currie, R. A. and Alessi, D. R. (2000) Identification of a pocket in the PDK1 kinase domain that interacts with PIF and the C-terminal residues of PKA. Embo J., 19, 979-988.
Biondi, R. M., Kieloch, A., Currie, R. A., Deak, M. and Alessi, D. R. (2001) The PIF-binding pocket in PDK1 is essential for activation of S6K and SGK, but not PKB. Embo J., 20, 4380-4390.
Frodin, M., Antal, T. L., Dummler, B. A., Jensen, C. J., Deak, M., Gammeltoft, S, and Biondi, R. M. (2002) A phosphoserine/threonine-binding pocket in AGC kinases and PDK1 mediates activation by hydrophobic motif phosphorylation. Embo J., 21, 5396-5407.
Frodin, M., Jensen, C. J., Merienne, K. and Gammeltoft, S. (2000) A phosphoserine-regulated docking site in the protein kinase RSK2 that recruits and activates PDK1. Embo J., 19, 2924-2934.
Goodford, P. J. (1985) A computational procedure for determining energetically favorable binding sites on biologically important macromolecules. J Med Chem, 28, 849-857.
Jensen, C. J., Buch, M.-B., Krag, T. O., Hemmings, B. A., Gammeltoft, S. and Frodin, M. (1999) 90-kDa Ribosomal S6 Kinase Is Phosphorylated and Activated by 3-Phosphoinositide-dependent Protein Kinase-1. J. Biol. Chem., 274, 27168-27176.
Jorgensen, T. J., Gardsvoll, H., Dano, K., Roepstorff, P. and Ploug, M. (2004) Dynamics of urokinase receptor interaction with Peptide antagonists studied by amide hydrogen exchange and mass spectrometry. Biochemistry, 43, 15044-15057.
Kohn, A. D., Kovacina, K. S. and Roth, R. A. (1995) Insulin stimulates the kinase activity of RAC-PK, a pleckstrin homology domain containing ser/thr kinase. Embo J, 14, 4288-4295.
Laskowski, R. A., Moss, D. S. and Thornton, J. M. (1993) Main-chain bond lengths and bond angles in protein structures. J Mol Biol, 231, 1049-1067.
Pullen, N., Dennis, P. B., Andjelkovic, M., Duiher, A., Kozma, S. C., Hemmings, B. A. and Thomas, G. (1998) Phosphorylation and activation of p70s6k by PDK1. Science, 279, 707-710.
Ramaswamy, S., Nakamura, N., Vazquez, F., Batt, D. B., Perera, S., Roberts, T. M. and Sellers, W. R. (1999) Regulation of G1 progression by the PTEN tumor suppressor protein is linked to inhibition of the phosphatidylinositol 3-kinase/Akt pathway. Proc Natl Acad Sci USA, 96, 2110-2115.
Weis, D. D., Engen, J. R. and Kass, I. J. (2006) Semi-Automated Data Processing of Hydrogen Exchange Mass Spectra Using HX-Express. J Am Soc Mass Spectrom, 17, 1700-1703.
Yang, J., Cron, P., Good, V. M., Thompson, V., Hemmings, B. A. and Barford, D. (2002a) Crystal structure of an activated Akt/protein kinase B ternary complex with GSK3-peptide and AMP-PNP. Nat Struct Biol, 9, 940-944.
Zhao, Y., Bjorbak, C. and Moller, D. E. (1996) Regulation and Interaction of pp 90rsk Isoforms with Mitogen-activated Protein Kinases. J. Biol. Chem., 271, 29773-29779.

TABLE I
Effect of mutations in PDK1 on basal activity and
activation by HM polypeptide PIFtide and compound 1

| | | | PIFtide | | Compound 1 | |
|---|---|---|---|---|---|---|
| | Basal | | | Max. | | Max. |
| | activity | | AC₅₀ | activity | AC₅₀ | activity |
| | (U/mg) | (%) | (μM) | (U/mg) | (μM) | (U/mg) |
| GST-PDK1 wt | 0.2 | 100 | 0.11 ± 0.03 | 0.9 ± 0.05 | 34 ± 9 | 1.1 ± 0.07 |
| GST-PDK1 [Ile119Ala] | 0.65 | 331 | 0.66 ± 0.14 | 2.58 ± 0.17 | 90 ± 20 | 2.1 ± 0.23 |
| GST-PDK1 [Val124Ala] | 0.48 | 241 | 0.21 ± 0.03 | 1.91 ± 0.06 | >200 | 2.6 ± 0.5 |
| GST-PDK1 [Val124Leu] | 0.4 | 200 | 0.35 ± 0.2 | 0.59 ± 0.04 | 180 ± 60 | 1.1 ± 0.13 |
| GST-PDK1 [Val127Leu] | 0.6 | 301 | 0.12 ± 0.03 | 0.99 ± 0.08 | No Effect | — |
| GST-PDK1 [Val127Thr] | 1.2 | 597 | 0.14 ± 0.07 | 2.36 ± 0.15 | 104 ± 58 | 1.69 ± 0.2 |
| GST-PDK1 [Arg131Ala] | 0.72 | 358 | 3.7 ± 2 | 1.24 ± 0.8 | >200 | 1.09 ± 0.06 |
| GST-PDK1 [Arg131Met] | 0.2 | 101 | 0.29 ± 0.05 | 0.34 ± 0.06 | No Effect | — |
| GST-PDK1 [Arg131Lys] | 0.3 | 149 | 0.13 ± 0.02 | 0.77 ± 0.02 | 51 ± 15 | 1.13 ± 0.09 |
| GST-PDK1 [Ser135Ala] | 0.89 | 443 | 0.18 ± 0.02 | 3.4 ± 0.09 | 109 ± 33 | 2.82 ± 0.4 |
| GST-PDK1 [Thr148Val] | 0.59 | 295 | INH | NA | 102 ± 19 | 2.44 ± 0.26 |
| GST-PDK1 [Gln150Ala] | 0.3 | 150 | 0.5 ± 0.06 | 1.55 ± 0.06 | 55 ± 9 | 1.12 ± 0.06 |
| GST-PDK1 [Gln150Glu] | 0.57 | 285 | No Effect | — | >200 | 2.5 ± 0.7 |
| GST-PDK1 [Gln150Lys] | 0.36 | 182 | 0.29 ± 0.05 | 2.7 ± 0.2 | 15 ± 4 | 1.37 ± 0.06 |
| GST-PDK1 [Gln150Met] | 0.61 | 305 | 0.86 ± 0.25 | 2.11 ± 0.15 | 177 ± 64 | 2.28 ± 0.26 |
| GST-PDK1 [Leu155Glu] | 0.60 | 300 | No Effect | — | No Effect | — |
| GST-PDK1 [Leu155Ser] | 2.75 | 915 | No Effect | — | No Effect | — |
| GST-PDK1 [Leu155Val] | 1.46 | 732 | No Effect | — | No Effect | — |
| GST-PDK1 [Leu155Ala] | 1.05 | 525 | 3.7 ± 0.66 | 3.46 ± 0.15 | >200 | 2.02 ± 0.61 |
| GST-PDK1 [Phe157Leu] | 0.7 | 349 | 0.32 ± 0.19 | 1.4 ± 0.14 | ND | ND |
| GST-PDK1 [Phe157Met] | 0.5 | 250 | 1.52 ± 0.59 | 1.7 ± 0.24 | 140 ± 100 | 0.9 ± 0.3 |
| GST-PDK1 [Lys115Met] | 0.32 | 160 | No Effect | — | >200 | NA |
| PDK1 50-360 | 0.65 | 325 | 0.17 ± 0.09 | 2.62 ± 0.18 | 38 ± 8 | 3.46 ± 0.19 |
| PDK1 50-360 [CT-PIF] | 1.02 | 510 | No Effect | — | No Effect | — |

PDK1 activity was measured at room temperature using a polypeptide substrate derived from the activation loop phosphorylation site of PKB, T308tide (0.1 mM). The listed data indicate the basal activity of the kinases (U/mg), their relative activity as a comparison to GST-PDK1 wt (%), AC₅₀ (μM) as well as the maximal activity in the presence of PIFtide and compound 1 (U/mg). GST-PDK1 wt was purified in numerous occasions and the maximal activation level of different batches varied between 3 and 8 fold activation with PIFtide or compound 1. Variations in the basal activity and fold activation were also observed in mutants. Therefore, comparisons in the maximal level of activation between mutants should be performed with care. The data shown are from an experiment where PIFtide concentrations were tested between 50 nM and 20 □M and compound 1 concentrations were between 5 μM and 200 μM, in triplicate. AC₅₀ and max. activity were estimated by fitting the data to a hyperbola using Kaleidagraph software. One Unit of PDK1 activity was defined as the amount required to catalyse the phosphorylation of 1 nmol of the T308tide in 1 min. ND, not determined; No Effect, addition of PIFtide or compound 1 did not have any effect on the basal activity (−) of the mutant kinase; INH, the addition of PIFtide to this mutant inhibited its activity; NA, the data could not be accurately estimated within the concentrations tested.

TABLE II
Specificity of low molecular weight compounds towards PDK1 and a panel of AGC
kinases. Basic scaffold indicating positions of R1, R2, R3 and R4; Effect of R1, R2, R3 and R4
on the activity of small compounds.

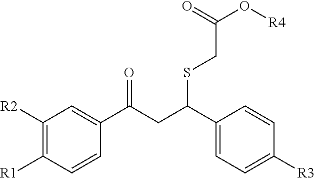

| | | | PDK1 | S6K1 | PKBα | SGK1 | PKCξ | PRK2 | PKA |
|---|---|---|---|---|---|---|---|---|---|
| Substitutions | Compnd | (μM) | (%) | (%) | (%) | (%) | (%) | (%) | (%) |
| R1 = Cl; R2 = H; | 1 | 20 | 234 | 109 | 89 | 97 | 94 | 103 | 100 |
| R3 = H; R4 = H | | 200 | 370 | 110 | 85 | 61 | 21 | 110 | 94 |
| R1 = Cl; R2 = H; | 2 | 20 | 109 | 109 | 92 | 89 | 104 | 106 | 109 |
| R3 = H; | | | | | | | | | |
| R4 = CH3 | | 200 | 124 | 120 | 106 | 92 | 86 | 89 | 103 |
| R1 = Cl; R2 = Cl; | 3 | 20 | 223 | 101 | 89 | 74 | 62 | 97 | 109 |
| R3 = H; R4 = H | | 200 | 381 | 98 | 73 | 48 | 2 | 104 | 81 |
| R1 = H; R2 = Cl; | 4 | 20 | 110 | 93 | 107 | 94 | 95 | 99 | 91 |
| R3 = H; R4 = H | | 200 | 190 | 104 | 104 | 72 | 64 | 96 | 77 |
| R1 = Cl; R2 = Cl; | 5 | 20 | 109 | 105 | 108 | 99 | 104 | 106 | 102 |
| R3 = Cl; R4 = H | | 200 | 131 | 130 | 122 | 83 | 88 | 100 | 106 |

Activity assays for each kinase were performed as described in Materials and Methods in the presence of 1% DMSO (100%). Except PKA, all protein kinases were produced as a GST-fusion. S6K1, PKBα, SGK mutants used were S6K1-T2-[Thr412Glu], PKBa[Ser473Asp] and ΔN-SGK[Ser422Asp].

1. A method of identifying or validating a compound that modulates the phosphorylation-dependent activity of a target protein or protein complex, where the target protein or protein complex activity is regulated by phosphorylation, and where the target protein or protein complex contains at least two interaction sites, one phosphate binding site and a separate target site, wherein polypeptide interaction to the interaction sites are regulated by phosphorylation, and the ability of a compound to inhibit, promote or mimic the interaction to the target site is measured and a compound that inhibits, promotes or mimics the said interaction is selected, whereas when the target protein is an AGC kinase, the polypeptide interacting to the target site does not comprise the sequence Phe/Tyr-Xaa-Xaa-Phe/Tyr or comprises a mutation equivalent to Val127Leu in PDK1.

2. The method of claim 1 where the phosphorylation-dependent activity of the target protein or protein complex can be mimicked by a polypeptide comprising a phosphorylated site, wherein the polypeptide contains additional sequences which promote the binding to the target protein at the target site.

3. The method of claim 2 where the phosphorylated polypeptide and the polypeptide interacting with the target site are different polypeptides.

4. The method of claim 1 where the target site is the HM/PIF-pocket binding site on PDK1 and the assay is performed with a mutant PDK1 which has mutated Val127 to Leu.

5. A method of identifying or validating a compound that modulates the phosphorylation-dependent activity of a target protein or protein complex, where the target protein or protein complex activity is regulated by phosphorylation, and where the target protein is an AGC kinase mutated to Leu at the site equivalent to Val127 and the ability of a compound to inhibit, promote or mimic the interaction to the target site is measured and a compound that inhibits, promotes or mimics the said interaction on the wild tune AGC kinase but not in the mutated AGC kinase is selected.

6. The method of claim 5 where the identification or validation of a compound is tested on a cell system or animal model system genetically engineered to possess an AGC kinase gene mutated at the equivalent site to PDK1 Val 127.

7. The method of claim 5 where the identification or validation of a compound involves the testing of the said compound on a mouse cell system or mouse animal model genetic engineered to contain one or two copies of PDK1 gene mutated at Val 127 site.

8. The method of claim 5 where the effect of the compound is tested as a comparison to the effects of the compound on the cell system or animal model which do not have this mutation.

9. The method of claim 5 where the effect of the compound is tested on a cancer system.

10. The method of claim 5 any claim 58 where the cell system or animal model tested contains the said PDK1 127Leu mutation and in addition at least one other mutation which predisposes the organism to disease.

11. The method of claim 10 where the disease is cancer.

12. The method of claim 10 where the other mutation is present on the gene coding for PTEN lipid phosphatase or on the gene coding for PKB/Akt protein kinase.

13. A method to activate kinases by mimicking the conformational transitions physiologically triggered by phospho-peptide docking.

14. The method according to claim 13 wherein the kinase is PDK1 or wherein the kinase is an isoform of PKB/Akt, MSK, S6K, PKC, RSK or PRK and the phosphorylated polypeptide comprises the sequence of an AGC kinase region comprising hydrophobic residues and the site equivalent to the “turn-motif”/Z-phosphorylation site.

15. The method according to claim 1 wherein the polypeptide possesses a Glutamic acid or Aspartic acid at a phosphorylation site.

16. A compound according to formula I, 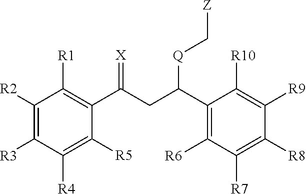
in which X is selected from O, N—R, or NO—R, R is H, C1-C4-alkyl, or -L-Y, wherein L is a linker and Y is a functional group, Q is selected from S or CH₂, Z is selected from COOH, tetrazolyl, nitril, phosphonic acid, phosphate, or COOE, in which E is C1-C5-alkanoyloxy-C1-C3-alkyl or C1-C-alkoxycarbonyloxy-C1-C3-alkyl, and R1, R4-R10 is selected from H, halogen, C1-C4-alkyl, C2-C4-alkenyl, or trifluoromethyl, and R2, R3 are either member of benzoanneleted cyclopentane, cyclohexane or benzene or are independently selected from H, halogen, C1-C4-alkyl, C2-C4-alkenyl, or trifluoromethyl.

17. The compound according to claim 16, in which R1, R4-R7 and R10 is selected from H or F, and R2, R3, R8 and R9 are selected from H, halogen, C1-C4-alkyl, C2-C4-alkenyl, or trifluoromethyl, and at least one of R2, R3, R8 or R9 is not H.

18. The compound according to claim 17, in which X is selected from O or N—OH, and Z is selected from COOH or COOE, in which E is C1-C5-alkanoyloxy-C1-C3-alkyl or C1-C-alkoxycarbonyloxy-C1-C3-alkyl.

19. The compound according to claim 18, in which R1, R4-R7, R9 and R10 is H.

20. The compound according to claim 19, in which X is O.

21. The compound according to claim 19, in which E is selected from acetoxymethyl, propionyloxymethyl, isopropionyloxymethyl, N-butyryloxymethyl, isobutyryloxymethyl, 2,2-dimethylpropionyloxymethyl, isovaleryloxymethyl, 1-acetoxy-1-ethyl, 1-acetoxy-1-propyl, 2,2-dimethylpropionyloxy-1-ethyl, 1-methoxycarbonyloxy-1-ethyl, 1-ethoxycarbonyloxy-1-ethyl, 1-isopropoxycarbonyloxyethyl or methoxycarbonyloxymethyl.

22. A compound of general formula II 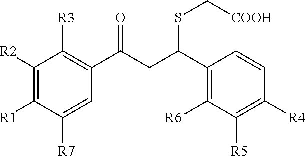
in which R1-R7 have the meanings indicated in the following table:

| Compound | |
|---|---|
| No. | |
| II.1 | R1 = Cl, R2-R7 = H |
| II.2 | R1 = Br, R2-R7 = H |
| II.3 | R1 = I, R2-R7 = H |
| II.4 | R1 = CF₃, R2-R7 = H |
| II.5 | R1 = CH₃, R2-R7 = H |
| II.6 | R1 = ethyl, R2-R7 = H |
| II.7 | R1 = propyl, R2-R7 = H |
| II.8 | R1 = isopropyl, R2- |
| | R7 = H |
| II.9 | R1 = Cl, R2 = Cl, R3- |
| | R7 = H |
| II.10 | R1 = H, R2 = Cl, R3- |
| | R7 = H |
| II.11 | R1 = Cl, R2 = Cl, R4 = Cl, |
| | R3 = R5 = R6 = R7 = H |
| II.12 | R1 = CF₃, R4 = Cl, |
| | R2 = R3 = R5 = R6 = R7 = H |
| II.13 | R1 = Cl, R4 = Cl, |
| | R2 = R3 = R5 = R6 = R7 = H |
| II.14 | R1 = Br, R4 = Cl, |
| | R2 = R3 = R5 = R6 = R7 = H |
| II.15 | R2 = I, R4 = Cl, |
| | R2 = R3 = R5 = R6 = R7 = H |
| II.16 | R1 = Cl, R4 = F, |
| | R2 = R3 = R5 = R6 = R7 = H |
| II.17 | R1 = Cl, R3 = Cl, R2 = H, R4- |
| | R7 = H |
| II.18 | R1 = H, R2 = H, R3 = Cl, R7 = Cl, |
| | R4-R6 = H |
| II.19 | R1 = Cl, R5 = Cl, R3-R4 = H, |
| | R6 = R7 = H |
| II.20 | R1 = Br, R5 = Cl, R3-R4 = H, |
| | R6 = R7 = H |
| II.21 | R1 = I, R5 = Cl, R3-R4 = H, |
| | R6 = R7 = H |
| II.22 | R1 = Cl, R2-R5 = H, R6 = F, |
| | R7 = H |
| II.23 | R1 = Br, R2-R5 = H, R6 = F, |
| | R7 = H |
| II.24 | R1 = I, R2-R5 = H, R6 = F, R7 = H |
| II.25 | R1 = Cl, R2-R5 = H, R6 = Cl, |
| | R7 = H |
| II.26 | R1 = Br, R2-R5 = H, R6 = Cl, |
| | R7 = H |
| II.27 | R1 = I, R2-R5 = H, R6 = Cl, R7 = H |
| II.28 | R1 = CF₃, R5 = Cl, R3-R4 = H, |
| | R6 = R7 = H |
| II.29 | 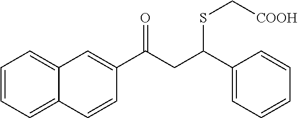 |
| II.30 | 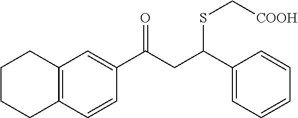 |
| II.31 | 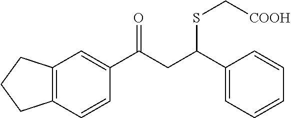 |

23. The use of the compound of claim 16 for co-crystallization with a protein.

24. The use of the compound of claim 16 for target validation studies.

25. The use of the compound of claim 16 as a lead compound for drug development, including virtual docking to target proteins.

26. The use of the compound of claim 16 for the production of a pharmaceutical preparation.

27. The use of the compound of claim 16 for the production of a pharmaceutical preparation for the treatment of cancer.

28. The use of the compound of claim 16 for the production of a pharmaceutical preparation for the treatment of insulin resistance and diabetes.

29. A method for rationale drug design characterized by the following steps:
1) selection of a kinase which is activated upon phosphorylation
2) comparing the structures of the activated and the inactivated form of said kinase and identifying one or more pockets
3) in-silico-screening of a compound library in order to find compound which match the pocket
4) confirmation of the results of step 3 by an in-vitro or in-vivo screen
5) optionally, optimization of hit compounds by comparing the compound with the pocket and subsequent derivatization in order to match the pocket.

30. A method of identifying or validating a compound that modulates the phosphorylation-dependent activity of a target protein or protein complex, where the target protein or protein complex activity is regulated by phosphorylation, and where the target protein or protein complex contains at least two interaction sites, one phosphate binding site and a separate target site, wherein polypeptide interaction to the interaction sites are regulated by phosphorylation, and the ability of a compound to inhibit, promote or mimic the interaction to the target site is measured and a compound that inhibits, promotes or mimics the said interaction is selected, whereas when the target protein is an AGC kinase the interacting polypeptide is phosphorylated at the Z-motif phosphorylation site and the phosphate binding site is comprised by at least one residue from the Z-phosphate binding site.

31. The method of claim 1 where the AGC kinase in the screening is mutated in the Z-phosphate binding site.

32. A diagnostic method in which a blood or tissue sample of a human being is taken and examined for AGC kinase mutations at the z-phosphate binding site.

33. The diagnostic method of claim 32 in which the AGC mutations are compared to mutation patterns of previously examined human beings.

34. The method according to claim 32 where the AGC kinase is an isoform of PKB.

35. The method according to claim 32 where the AGC kinase is an isoform of MSK.

36. A diagnostic method in which
i) blood or tissue sample from several healthy human beings as well as from cancer patients are screened for PKB or MSK mutations of a human being is taken and examined for PKB or MSK mutations,
ii) the mutation pattern of step i is stored in a database
iii) a blood or tissue sample of a human being is taken and examined for PKB or MSK mutations
iv) the result of iii is compared with data from the database in order to determine the human being's predisposition for cancer.

37. The diagnostic method according to claim 36 where the analysis is performed on a disease or infected tissue the result of the analysis is used to decide the treatment of the patient.

38. A method of identifying or validating a compound that modulates the phosphorylation-dependent activity of a target protein kinase, where the target protein kinase is regulated by phosphorylation, and where the target protein kinase contains at least two interaction sites, one phosphate binding site which binds to the “turn-motif” or “Z”-phosphate and a separate target site, wherein a polypeptide interaction to the interaction sites are regulated by phosphorylation, and the ability of a compound to inhibit, promote or mimic the interaction to the target site is measured and a compound that inhibits, promotes or mimics the said interaction is selected, whereas when the target protein is an AGC kinase, the kinase is not PDK1.

39. The method of claim 1 where the target protein kinase is mutated at one or more residues forming part of the “turn-motif” or “Z”-phosphorylation site and the ability of a compound to inhibit, promote or mimic the interaction to the target site is measured in the mutant target protein kinase and on the protein kinase which possesses Z-phosphate binding site residues different from those of the mutated Z-phosphate protein and a compound that inhibits, promotes or mimics the said interaction on the protein kinase which possesses Z-phosphate binding site residues different from those of the mutated Z-phosphate but not on the mutated target protein kinase is selected.

40. The method of claim 1, where the target protein or protein kinase binds a separate polypeptide comprising a phosphorylated “turn-motif” or “Z”-phosphate, and where the target protein kinase contains at least two interaction sites, one phosphate binding site which binds to the “turn-motif” or “Z”-phosphate and a separate target site, wherein the intra-molecular polypeptide interaction to the interaction sites are regulated by phosphorylation, and the ability of a compound to inhibit, promote or mimic the interaction to the target site is measured and a compound that inhibits, promotes or mimics the said interaction is selected, whereas when the target protein is an AGC kinase, the kinase is not PDK1.

41. A method of identifying or validating a compound that modulates the phosphorylation-dependent activity of a target protein kinase, where the target protein kinase is regulated by phosphorylation, and where the target protein kinase contains at least two interaction sites, one phosphate binding site which binds to the “turn-motif” or “Z”-phosphate and a separate target site, wherein the interaction of the “turn-motif” or “Z”-phosphate and a separate binding site on the target protein kinase are modelled or derived from structure data and a compound predicted to inhibit, promote or mimic the interaction to the phosphate binding site or target site is selected.

42. The method of claim 1 where the small compound predicted to partially inhibit, promote or mimic the interaction is in silico selected and chemically modified to further interact with other binding sites and increase potency of interaction on the target protein kinase.

43. A method of determining the therapy for a patient involving the identification of the aminoacid sequence of the residues forming part of the “turn-motif”/“Z”-phosphate binding site in AGC kinases.

44. The method of claim 6 involving the sequencing of the DNA corresponding to the “turn-motif”/“Z”-phosphate binding site in AGC kinases.

45. The method of claim 6 including the determination of the mutated “turn-motif”/“Z”-phosphate binding site employing specific antibodies or a proteomic approach.

46. The method of claim 1 when the AGC kinase isoform is an isoform of S6K, SGK, MSK, RSK, PKB.

47. The method of claim 6 when the AGC kinase is an isoform of PKB.

48. The method of claim 34 when the AGC kinase is an isoform of PKB and the identification of mutations in the “turn-motif”/“Z”-phosphate binding site are performed on DNA derived from cancer cells and the result of the analysis is used to determine a personalized therapy.

49. The method of claim 34 where the identification of a mutation “turn-motif”/“Z”-phosphate binding site in a PKB isoform in cancer determines that the personalized treatment will exclude cancer treatment with drugs targeting the signalling pathway upstream of PKB and that will include a drug which targets the PKB-dependent cell survival signalling pathway targeting PKB or targets downstream of PKB.

50. The method of claim 34 when the AGC kinase is an isoform of MSK and the identification of mutations in the “turn-motif”/“Z”-phosphate binding site are performed on DNA derived from cancer cells and the result of the analysis is used to determine a personalized therapy.

51. The method of claim 34 where the identification of a mutation “turn-motif”/“Z”-phosphate binding site in a MSK isoform in cancer determines that the personalized treatment will not include inhibitors or drugs targeting the pathway upstream of MSK1 but will include a drug targeting MSK or MSK-downstream targets.
